(19) United States
(12) Patent Application Publication
(10) Pub. No.: US 2024/0384292 A1
(43) Pub. Date: Nov. 21, 2024

(54) VECTORIZED TNF-ALPHA ANTAGONISTS FOR OCULAR INDICATIONS

(71) Applicant: REGENXBIO INC., Rockville, MD (US)

(72) Inventors: Xu Wang, Falls Church, VA (US); Devin McDougald, Boston, MA (US); Joseph Bruder, Gaithersburg, MD (US); Ye Liu, Clarksville, MD (US); Olivier Danos, Princeton, NJ (US); Chunping Qiao, Chapel Hill, NC (US); Wei-Hua Lee, Darnestown, MD (US)

(21) Appl. No.: 18/034,042

(22) PCT Filed: Oct. 29, 2021
(86) PCT No.: PCT/US21/57380

§ 371 Date: Apr. 26, 2023

Related U.S. Application Data
(60) Provisional application No. 63/107,415, filed on Oct. 29, 2020.

(51) Int. Cl. C12N 15/86; A61K 48/00; A61P 27/02; C07K 14/715

(52) U.S. Cl. CPC C12N 15/86; A61K 48/0058; A61P 27/02; C07K 14/7151; C07K 2319/30; C07K 2319/32; C12N 2750/14143; C12N 2750/14151

(57) ABSTRACT

Provided are methods and compositions for the delivery of fully human post-translationally modified therapeutic TNF receptor Fc fusions. The fully human post-translationally modified therapeutic TNF receptor Fc fusions may be delivered by gene therapy methods, e.g., as a recombinant adeno-associated virus (rAAV) vector to the appropriate tissue. Also provided are methods of treating ocular indications such as non-infectious uveitis with the fully human post-translationally modified therapeutic TNF receptor Fc fusions.

### 1. INTRODUCTION

Compositions and methods are described for the delivery of a fully human post-translationally modified (HuPTM) therapeutic tumor necrosis factor receptor (TNFR) fusion protein that binds to tumor necrosis factor alpha (TNF-α) to a human subject diagnosed with non-infectious uveitis.

### 2. BACKGROUND OF THE INVENTION

Therapeutic anti-TNFα Fc fusion proteins have been shown to be effective in treating a number of diseases and conditions. However, because these agents are effective for only a short period of time, repeated injections for long durations are often required, thereby creating considerable treatment burden for patients.

Uveitis includes a group of heterogeneous diseases characterized by inflammation of the uveal tract. Uveitis may be generally classified by the etiology of inflammation as infectious or non-infectious (autoimmune disorders), which could be related or not to a systemic disease. In addition, uveitis can be anatomically classified as anterior, intermediate, posterior or panuveitis, and they may have an acute, chronic or recurrent course. The clinical presentation is variable, the symptoms may include blurred vision, photophobia, ocular pain and significant visual impairment (Valenzuela et al., Front Pharmacol. 2020; 11: 655).

Non-infectious uveitis is a serious, sight-threatening intraocular inflammatory condition characterized by inflammation of the uvea (iris, ciliary body, and choroid). Non-infectious uveitis is thought to result from an immune-mediated response to ocular antigens and is a leading cause of irreversible blindness in working-age population in the developed world. The goal of uveitis treatment is to control inflammation, prevent recurrences, and preserve vision, as well as minimize the adverse effects of medications. Currently, the standard of care for non-infectious uveitis includes the administration of corticosteroids as first-line agents, but in some cases a more aggressive therapy is required. This includes synthetic immunosuppressants, such as antimetabolites (methotrexate, mycophenolate mofetil, and azathioprine), calcineurinic inhibitors (cyclosporine, tacrolimus), and alkylating agents (cyclophosphamide, chlorambucil). In those patients who become intolerant or refractory to corticosteroids and conventional immunosuppressive treatment, biologic agents have arisen as an effective therapy in pediatric and adulthood uveitis, based on targeting relevant immunological pathways involved in disease pathogenesis. Current immunomodulatory therapy includes the inhibition of TNFα, achieved with mAb, such as infliximab, adalimumab, golimumab, and certolizumab-pegol, or with TNF receptor fusion protein, etanercept. In this regard, anti-TNF agents (infliximab and adalimumab) have shown the strongest results in terms of favorable outcomes. Adalimumab has been approved for treatment of non-infectious uveitis by subcutaneous administration every other week. When conventional immunosuppressive treatments and/or anti-TNF-α therapies fail, other biological agents are recommended.

EYS606 encodes a recombinant fusion protein linking the TNFα p55 receptor 1 (TNFR1) to an Fc portion of an IgG1 immunoglobulin molecule. EYS606 is currently under investigation as non-viral gene therapy for the treatment of non-infectious posterior, intermediate or panuveitis. EYS606 is administered by electrotransfer in the ciliary muscle of patients.

Etanercept is a dimeric fusion protein consisting of two extracellular domains of human p75 TNFα receptor (TNFR2) fused to an Fc portion of an IgG1 immunoglobulin molecule (TNFR:Fc) and may be used for treatment of several autoimmune inflammatory diseases, including rheumatoid arthritis, Crohn's disease, and psoriatic arthritis. Etanercept is subcutaneously administrated at a dose of 50 mg once per week or 25 mg twice a week (Valenzuela et al., Front Pharmacol. 2020; 11: 655.).

There is a need for more effective treatments that reduce the treatment burden on patients suffering from non-infectious uveitis. Intravitreal medications have become a promising mode of drug administration in uveitis patients as they provide high volume of drug to the target tissues, eliminating the risk of systemic toxicity. Reducing or eliminating the need for periodic ocular administration would reduce patient burden and improve therapy.

### 3. SUMMARY OF THE INVENTION

Biologic inhibitors delivered by gene therapy have several advantages over injected or infused therapeutic biologic inhibitors that dissipate over time resulting in peak and trough levels. Sustained expression of the transgene product biologic inhibitor, as opposed to injecting a biologic inhibitors repeatedly, allows for a more consistent level of the biologic inhibitor to be present at the site of action, and is less risky and more convenient for patients, since fewer injections need to be made. Furthermore, biologic inhibitors expressed from transgenes are post-translationally modified in a different manner than those that are directly injected because of the different microenvironment present during and after translation. Without being bound by any particular theory, this results in biologic inhibitors that have different diffusion, bioactivity, distribution, affinity, pharmacokinetic, and immunogenicity characteristics, such that the antibodies delivered to the site of action are “biobetters” in comparison with directly injected biologics. Systemic administration, such as expressed from a depot in the liver, or, in addition, localized delivery of the therapeutic to eye tissue could reduce exposure of the subject to the therapeutic and reduce the risk of potential adverse effects. Accordingly, provided herein are compositions and methods for anti-TNFα gene therapy, particularly recombinant AAV gene therapy, designed to target the eye and generate a depot of transgenes for expression of anti-TNFα Fc fusion proteins (TNFR:Fc), particularly etanercept (TNFR2:Fc) and EYS606 (TNFR1:Fc), that result in a therapeutically effective or prophylactic levels of the biologic inhibitor in the eye within 20 days, 30 days, 40 days, 50 days, 60 days, or 90 days of administration of the rAAV composition.

Compositions and methods are described for the systemic delivery of an anti-TNFα Fc fusion protein to a patient (human subject) diagnosed with non-infectious uveitis or other condition indicated for treatment with the therapeutic anti-TNFα Fc fusion protein. Delivery may be advantageously accomplished via gene therapy—e.g., by administering a viral vector or other DNA expression construct encoding a therapeutic anti-TNFα Fc fusion protein to a subject diagnosed with a condition indicated for treatment with the therapeutic anti-TNFα Fc fusion protein—to create a permanent depot in the eye, or in alternative embodiments, liver and/or muscle, of the patient that continuously supplies the HuPTM anti-TNFα Fc fusion protein, e.g., a human-glycosylated transgene product, to the appropriate eye tissue (including via the circulation circulation) of the subject where the anti-TNFα Fc fusion protein exerts its therapeutic or prophylactic effect.

The recombinant vector used for delivering the transgene includes non-replicating recombinant adeno-associated virus vectors (“rAAV”). In embodiments, the AAV type has a tropism for the eye, for example, AAV8 subtype, AAV3B subtype, AAV2.7m8, or AAVrh73 subtype of AAV However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements, particularly elements that are ocular tissue-specific control elements, for example one or more elements of Table 1 or, alternatively, Table 1a. The constructs also may comprise a signal or leader peptide that directs secretion of the expressed anti-TNFα Fc fusion protein, for example, a peptide from Table 2, or Tables 3 or 4.

In certain embodiments, the HuPTM anti-TNFα Fc fusion protein encoded by the transgene can include, but is not limited to, a TNFR:Fc fusion proteins that binds to TNFα, particularly etanercept (or any biosimilar version thereof) or EYS606, see, for example FIGS. 2A and 2B.

Gene therapy constructs for the therapeutic anti-TNFα Fc fusion protein are designed such that the soluble TNFR (TNFR1 or TNFR2) fused to the Fc portion are expressed as a dimeric fusion protein (SEQ ID NO: 10 and 12).

In addition, anti-TNFα Fc fusion proteins expressed from transgenes in vivo are not likely to contain degradation products associated with biologics produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

The production of HuPTM anti-TNFα Fc fusion proteins in the eye of the human subject should result in a “biobetter” molecule for the treatment of disease accomplished via gene therapy—e.g., by administering a viral vector or other DNA expression construct encoding a HuPTM anti-TNFα fusion protein to a patient (human subject) diagnosed with a disease indication for that Fc fusion protein, to create a permanent depot in the subject that continuously supplies the human-glycosylated, sulfated transgene product produced by the subject's transduced cells. The cDNA construct for the HuPTM anti-TNFα Fc fusion protein should include a signal peptide that ensures proper co- and post-translational processing (glycosylation and protein sulfation) by the transduced human cells.

As an alternative, or an additional treatment to gene therapy, the HuPTM anti-TNFα Fc fusion protein can be produced in human cell lines by recombinant DNA technology, and the glycoprotein can be administered to patients.

Combination therapies involving systemic delivery of the HuPTM anti-TNFα fusion protein to the patient accompanied by administration of other available treatments are encompassed by the methods provided herein. The additional treatments may be administered before, concurrently or subsequent to the gene therapy treatment. Such additional treatments can include but are not limited to co-therapy with the therapeutic anti-TNFα Fc fusion proteins or anti-TNFα mAbs.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

Provided are compositions comprising rAAV vectors which comprise an optimized expression cassette containing a photoreceptor-specific promoter or other suitable promoter, including a CAG promoter, Best1/GRK promoter (or other promoter as provided in Table 1 or Table 1a (and a codon optimized and CpG depleted transgene that express a transgene, for example, a soluble TNFR (TNFR1 or TNFR2) and Fc domain (including the hinge, CH2 and CH3 domains) of an anti-TNFα therapeutic Fc fusion protein, including etanercept and EYS606. Methods of administration and manufacture are also provided. In particular, provided are compositions and methods comprising rAAV comprising the construct CAG.etanercept or U1a.Vh4i.etanercept.scAAV (see Table 7).

Pharmaceutical compositions suitable for administration to human subjects comprise a suspension of the recombinant vector in a formulation buffer comprising a physiologically compatible aqueous buffer, a surfactant and optional excipients.

### 3.1 ILLUSTRATIVE EMBODIMENTS

1. A pharmaceutical composition for treating non-infectious uveitis in a human subject in need thereof, comprising an adeno-associated virus (AAV) vector having:
   (a) a viral capsid that has a tropism for ocular tissue cells; and
   (b) an artificial genome comprising an expression cassette flanked by AAV internal tandem repeats (ITRs), wherein the expression cassette comprises a transgene encoding an anti-TNFα Fc fusion protein, operably linked to one or more regulatory sequences that control expression of the transgene in human ocular cells;
wherein said AAV vector is formulated for subretinal, intravitreal, intranasal, intracameral, suprachoroidal, or systemic administration to said human subject.
2. The pharmaceutical composition of paragraph 1, wherein the anti-TNFα Fc fusion protein comprises a soluble, extracellular portion of human TNFα receptor type I (TNFR1) or type II (TNFR2) covalently linked through a peptide bond to a polypeptide comprising an Fc domain of an immunoglobulin heavy chain.
3. The pharmaceutical composition of paragraph 1 or 2, wherein the viral capsid is at least 95% identical to the amino acid sequence of AAV serotype 1 (AAV1), serotype 2 (AAV2), serotype AAV2.7m8 (AAV2.7m8), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), serotype rh74 (AAVrh74), serotype hu51 (AAV.hu51), serotype hu21 (AAV.hu21), serotype hu12 (AAV.hu12), or serotype hu26 (AAV.hu26).
4. The pharmaceutical composition of any of paragraphs 1 to 3, wherein the AAV capsid is AAV8, AAV3B, AAV2.7m8, or AAVrh73.
5. The pharmaceutical composition of paragraphs 1 to 4, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
6. The pharmaceutical composition of paragraphs 1 to 5, wherein the regulatory sequence includes a regulatory sequence from Table 1 or 1b.
7. The pharmaceutical composition of paragraph 5, wherein the regulatory sequence is a human rhodopsin kinase (GRK1) promoter (SEQ ID NOS:54 or 117), a mouse cone arresting (CAR) promoter (SEQ ID NOS:114-116), a human red opsin (RedO) promoter (SEQ ID NO:112) a CB promoter (SEQ ID NO: 159), a CBLong promoter (SEQ ID NO: 160) or Best1/GRK promoter (SEQ ID NO:161).
8. The pharmaceutical composition of any of paragraphs 1 to 7, wherein the transgene encodes a signal sequence at the N-terminus of the anti-TNFα Fc fusion protein that directs secretion and post translational modification in said human ocular cells.
9. The pharmaceutical composition of paragraph 8, wherein said signal sequence is MYRMQLLLLIALSLALVTNS (SEQ ID NO:62) or a signal sequence from Table 2, 3 or 4.
10. The pharmaceutical composition of any of paragraphs 1 to 8, wherein transgene has the structure from the N-terminus to the C-terminus: Signal sequence-soluble TNFR extracellular domain—hinge region—Fc domain—PolyA.
11. The composition of paragraph 10, wherein the transgene further comprises a thrombin cleavage site of SEQ ID NO:8 at the C-terminus of the soluble TNFR extracellular domain.
12. The pharmaceutical composition of any of paragraphs 1 to 11, wherein anti-TNFα Fc fusion protein is expressed as a dimeric fusion protein.
13. The pharmaceutical composition of any of paragraphs 1 to 12, wherein the anti-TNFα Fc fusion protein is etanercept or EYS606.
14. The pharmaceutical composition of any of paragraphs 1 to 13, wherein said transgene comprises the nucleotide sequence of SEQ ID NO: 15-18.
15. The pharmaceutical composition of any of paragraphs 1 to 14, wherein the anti-TNFα Fc fusion protein has an amino acid sequence of SEQ ID NO: 10 or 12.
16. The pharmaceutical composition of any of paragraphs 1 to 15, wherein the anti-TNFα Fc fusion protein is a hyperglycosylated mutant or wherein the Fc polypeptide of the anti-TNFα Fc fusion protein is glycosylated or aglycosylated.
17. The pharmaceutical composition of any of paragraphs 1 to 16, wherein the Fc domain is an IgG1-Fc domain.
18. The pharmaceutical composition of any of paragraphs 1 to 17, wherein the artificial genome is self complementary.
19. The pharmaceutical composition of any of paragraphs 1 to 18 wherein the artificial genome is the construct CAG.etanercept (SEQ ID NO: 15 or 16) or mUla.Vh4i.etanercept.scAAV (SEQ ID NO: 17 or 18).
20. A composition comprising an adeno-associated virus (AAV) vector having:
   a. a viral AAV capsid, that is optionally at least 95% identical to the amino acid sequence of AAV serotype 1 (AAV1), serotype 2 (AAV2), serotype AAV2.7m8 (AAV2.7m8), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), serotype rh74 (AAVrh74), serotype hu51 (AAV.hu51), serotype hu21 (AAV.hu21), serotype hu12 (AAV.hu12), or serotype hu26 (AAV.hu26).
   b. an artificial genome comprising an expression cassette flanked by AAV inverted terminal repeats (ITRs), wherein the expression cassette comprises a transgene encoding an anti-TNF-α Fc fusion protein comprising a human soluble TNFα receptor type I (TNFR1) or type II (TNFR2) extracellular domain covalently linked through a peptide bond to a polypeptide comprising an Fc domain of an immunoglobulin heavy chain, operably linked to one or more regulatory sequences that promote expression of the transgene in human ocular tissue cells; wherein the transgene encodes a signal sequence at the N-terminus of said anti-TNFα Fc fusion protein that directs secretion and post translational modification of said anti-TNFα Fc fusion in ocular tissue cells.
21. The composition of paragraph 20, wherein the transgene further comprises a thrombin cleavage site of SEQ ID NO:8 at the C-terminus of soluble human TNFR extracellular domain.
22. The composition of any of paragraphs 20 or 21, wherein the anti-TNFα Fc fusion protein has an amino acid sequence of SEQ ID NO: 10 or 12.
23. The composition of paragraphs 20 to 22, wherein the anti-TNFα Fc fusion protein is etanercept or EYS606.
24. The composition of paragraphs 20 to 23, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
25. The composition of paragraphs 20 to 24, wherein said transgene comprises the nucleotide sequence of SEQ ID NO:13 or 14.
26. The composition of paragraphs 20 to 25, wherein said signal sequence is MYRMQLLLLIALSLALVTNS (SEQ ID NO:62) or a signal sequence from Tables 2, 3 or 4
27. The composition of any of paragraphs 20 to 26, wherein the artificial genome is self complementary.
28. The composition of any of paragraphs 20 to 27 wherein the artificial genome is the construct CAG.etanercept (SEQ ID NO: 15) or mUla.Vh4i.etanercept.scAAV (SEQ ID NO: 17).

### Method of Treatment

29. A method of treating non-infectious uveitis in a human subject in need thereof, comprising subretinally, intravitreally, intranasally, intracamerally, suprachoroidally, or systemically administering to the subject a therapeutically effective amount of a composition comprising a recombinant AAV comprising a transgene encoding an anti-TNFα Fc fusion protein operably linked to one or more regulatory sequences that control expression of the transgene in ocular tissue cells.
30. A method of treating non-infectious uveitis in a human subject in need thereof, comprising: subretinally, intravitreally, intranasally, intracamerally, suprachoroidally, or systemically administering to said subject a therapeutically effective amount of a recombinant nucleotide expression vector comprising a transgene encoding an anti-TNFα Fc fusion protein, operably linked to one or more regulatory sequences that control expression of the transgene in human ocular tissue cells, so that a depot is formed that releases a human post-translationally modified (HuPTM) form of anti-TNFα Fc fusion protein.
31. The method of paragraphs 29 and 30, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
32. The method of paragraphs 29 to 31, wherein said anti-TNFoa Fc fusion protein comprises a human soluble TNFα receptor type I (TNFR1) or type II (TNFR2) extracellular domain covalently linked to an IgG Fc domain.
33. The method of paragraphs 29 to 32, wherein said transgene is etanercept or EYS606.
34. The method of paragraphs 29 to 33, wherein said transgene has the nucleotide sequence of SEQ ID NO: 15-17.
35. The method of paragraphs 29 to 34, wherein the recombinant nucleotide expression vector is packaged in an rAAV having a viral capsid, wherein viral capsid is at least 95% identical to the amino acid sequence of AAV serotype 1 (AAV1), serotype 2 (AAV2), serotype AAV2.7m8, serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).
36. The method of any of paragraphs 29, 31 to 35, wherein the AAV capsid is AAV8, AAV2.7m8, AAV3B, or AAVrh73.
37. The method of any of paragraphs 29 to 36, wherein the regulatory sequence includes a regulatory sequence from Table 1 or 1a.
38. The method of paragraph 37, wherein the regulatory sequence is a human rhodopsin kinase (GRK1) promoter (SEQ ID NOS:54 or 117), a mouse cone arresting (CAR) promoter (SEQ ID NOS:114-116), a human red opsin (RedO) promoter (SEQ ID NO:112), a CB promoter (SEQ ID NO: 159), a CBLong promoter (SEQ ID NO: 160) or Best1/GRK promoter (SEQ ID NO: 161).
39. The method of any of paragraphs 29 to 38, wherein the transgene encodes a signal sequence at the N-terminus of the anti-TNFα Fc fusion protein that directs secretion and post translational modification in said human ocular tissue cells.
40. The method of paragraph 39, wherein said signal sequence is MYRMQLLLLIALSLALVTNS (SEQ ID NO:62) or a signal sequence from Tables 2, 3 or 4.
41. The method of any of paragraphs 25 to 40, wherein transgene has the structure: Signal sequence-9human soluble TNFR extracellular domain (type 1 or type 2)—hinge region—Fc domain—PolyA.
42. The method of paragraph 41, wherein the transgene further comprises a thrombin cleavage site having SEQ ID NO:8 at the C-terminus of the human soluble TNFR extracellular domain.
43. The method of any of paragraphs 29 to 42, wherein the anti-TNFα Fc fusion protein is a hyperglycosylated mutant or wherein the Fc domain of the anti-TNFα Fc fusion protein is glycosylated or aglycosylated.
44. The method of any of paragraphs 29 to 43, wherein anti-TNFα Fc fusion protein contains an alpha 2,6-sialylated glycan.
45. The method of any of paragraphs 29 to 44, wherein anti-TNFα Fc fusion protein is glycosylated but does not contain detectable NeuGc and/or α-Gal.
46. The method of any of paragraphs 29 to 45, wherein anti-TNFα Fc fusion protein contains a tyrosine sulfation.
47. The method of any of paragraphs 29 to 46 in which production of said HuPTM form of the anti-TNFα Fc fusion protein is confirmed by transducing human ocular cells in culture with said recombinant nucleotide expression vector and expressing said anti-TNFα Fc fusion protein.
48. The method of paragraphs 29 or 47, wherein the therapeutically effective amount is determined to be sufficient to improve best corrected visual acuity (BCVA) by >=2 ETDRS lines or increase in log MAR, reduced inflammatory activity of the anterior and posterior chamber according to the SUN classification, and/or reduction in grade of vitreous haze.
49. The method of any of paragraphs 29 to 48, wherein the rAAV is self complementary.
50. The method of any of paragraphs 29 to 49 wherein the transgene is within the construct CAG.etanercept (SEQ ID NO: 15 or 16) or mUla.Vh4i.etanercept.scAAV (SEQ ID NO: 17 or 18).

### Method of Manufacture

51. A method of producing recombinant AAVs comprising:
   (a) culturing a host cell containing:
      (i) an artificial genome comprising a cis expression cassette flanked by AAV ITRs, wherein the cis expression cassette comprises a transgene encoding an anti-TNFα Fc fusion protein, wherein the anti-TNFα Fc fusion protein comprises a soluble, extracellular portion of human TNFα receptor type I (TNFR1) or type II (TNFR2) covalently linked through a peptide bond to a polypeptide comprising an Fc domain of an immunoglobulin heavy chain, operably linked to one or more regulatory sequences that promote expression of the transgene in human ocular tissue cells;
      (ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue tropism;
      (iii) sufficient adenovirus helper functions to permit replication and packaging of the artificial genome by the AAV capsid protein; and
   (b) recovering recombinant AAV encapsidating the artificial genome from the cell culture.
52. The method of paragraph 51, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
53. The method of paragraphs 51 or 52, wherein the transgene encodes etanercept or EYS606, wherein the AAV capsid protein is The rAAV capsid protein of embodiment 1, wherein said capsid protein is from at least one AAV serotype of AAV serotype 1 (AAV1), serotype 2 (AAV2), serotype 2.7m8, serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), serotype rh74 (AAVrh74), serotype hu51 (AAV.hu51), serotype hu21 (AAV.hu21), serotype hu12 (AAV.hu12), or serotype hu26 (AAV.hu26).
54. A host cell containing:
   a. an artificial genome comprising a cis expression cassette flanked by AAV ITRs, wherein the cis expression cassette comprising a transgene encoding an anti-TNFα fusion protein, wherein the anti-TNFα Fc fusion protein comprises a soluble, extracellular portion of human TNFα receptor type I (TNFR1) or type II (TNFR2) covalently linked through a peptide bond to polypeptide comprising an Fc domain of an immunoglobulin heavy chain, operably linked to one or more regulatory sequences that promote expression of the transgene in human ocular tissue cells;
   b. a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue tropism;
   c. sufficient adenovirus helper functions to permit replication and packaging of the artificial genome by the AAV capsid protein.
55. The host cell of paragraph 54, wherein the transgene encodes etanercept or EYS606.
56. The host cell of paragraphs 53 or 54, wherein the AAV capsid protein is AAV serotype 1 (AAV1), serotype 2 (AAV2), serotype 2.7m8 (AAV2.7m8), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), serotype rh74 (AAVrh74), serotype hu51 (AAV.hu51), serotype hu21 (AAV.hu21), serotype hu12 (AAV.hu12), or serotype hu26 (AAV.hu26).

### 4. BRIEF DESCRIPTION OF THE DRAWINGS

FIG. 1. A schematic of an rAAV vector genome construct containing an expression cassette encoding etanercept controlled by expression elements, flanked by the AAV ITRs.

FIGS. 2A and 2B. The amino acid sequence of anti-TNFα Fc fusion proteins etanercept (A) and EYS606 (B). Glycosylation sites are boldface or underlined. Glutamine glycosylation sites; asparaginal (N) glycosylation sites, canonical and non-consensus asparaginal (N) glycosylation sites; and tyrosine-O-sulfation sites (italics) are as indicated in the legend. The Fc domain (CH2 and CH3 domains) is highlighted in grey. Hinge region is indicated in italics. Thrombin cleavage site is underlined.

FIG. 3. Clustal Multiple Sequence Alignment of various capsids with ocular tissue tropism. Amino acid substitutions (shown in bold in the bottom rows) can be made to AAV8 capsids by “recruiting” amino acid residues from the corresponding position of other aligned AAV capsids. Sequence shown in gray=hypervariable regions. The amino acid sequences of the AAV capsids are assigned sequence ID numbers as indicated in FIG. 3.

FIG. 4. Clustal Multiple Sequence Alignment of constant heavy chain regions (CH2 and CH3) of IgG1 (SEQ ID NO:47), IgG2 (SEQ ID NO:48), and IgG4 (SEQ ID NO:49). The hinge region, from residue 219 to residue 230 of the heavy chain, is shown in italics. The numbering of the amino acids is in EU-format.

FIGS. 5A and B. A. Binding of TNFα across model species (mouse, rat, and human) by vectorized adalimumab IgG and Fab. Negative control included supernatant derived from non-transfected cells. Vectorized lanadelumab (pAAV.CAG.lanadelumab.IgG) functioned as a non-specific antibody control. Data represented as mean±SEM. B. Binding of TNFα across model species (mouse, rat, and human) by vectorized adalimumab IgG and etanercept. Negative control included supernatant derived from non-transfected cells. Vectorized lanadelumab (pAAV.CAG.lanadelumab.IgG) functioned as a non-specific antibody control. Data represented as mean±SEM.

### 5. DETAILED DESCRIPTION OF THE INVENTION

Compositions and methods are described for the systemic for the delivery of a fully human post-translationally modified (HuPTM) therapeutic anti-TNFα Fc fusion protein that binds to TNFα to a human subject diagnosed with non-infectious uveitis. Delivery may be advantageously accomplished via gene therapy—e.g., by administering a viral vector or other DNA expression construct encoding a therapeutic anti-TNFα Fc fusion protein (or a hyperglycosylated derivative of either) to a patient (human subject) diagnosed with non-infectious uveitis to create a permanent depot in a tissue or organ of the patient, particularly ocular tissues such as the retina, that continuously supplies the HuPTM anti-TNFα Fc fusion protein, e.g., a human-glycosylated transgene product, to where the anti-TNFα Fc fusion protein exerts its therapeutic effect.

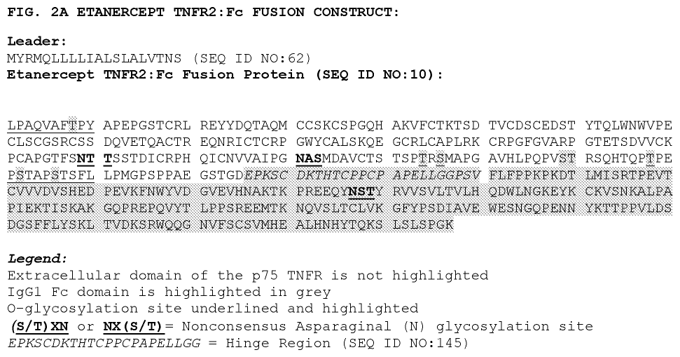

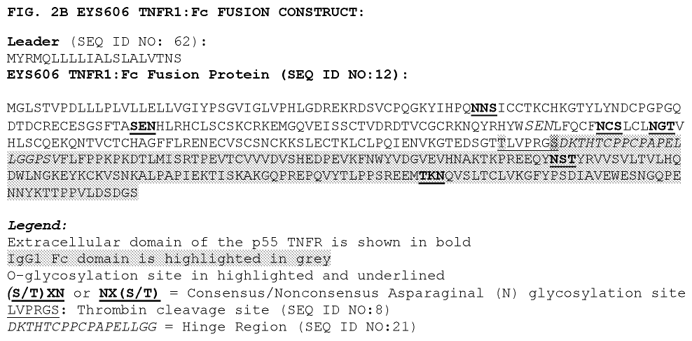

In certain embodiments, the HuPTM anti-TNFα Fc fusion protein encoded by the transgene, but it not limited to, is a TNFR:Fc fusion protein that binds TNFα, particularly etanercept or EYS606 (see FIGS. 2A and 2B for the amino acid sequence of the TNFR-Fc fusion protein etanercept and EYS606, respectively). In embodiments, when expressed, the TNFR Fc fusion protein is dimeric.

The HuPTM anti-TNFα Fc fusion protein encoded by the transgene can include, but is not limited to, a therapeutic anti-TNFα TNFR1:Fc fusion that binds to TNFα, including but not limited to, EYS606. The amino acid sequences of the foregoing are provided in Table 6, infra. The TNFR1 domain having an amino acid sequence of SEQ ID NO: 1 and Fc domain having an amino acid sequence of SEQ ID NO: 6 (or 7 without the hinge), and the EYS606 protein having an amino acid sequence of SEQ ID NO: 12. The HuPTM anti-TNFα TNFR1:Fc fusion encoded by the transgene can include, but is not limited to, a dimeric fusion protein engineered to contain additional glycosylation sites on the Fc domain.

The HuPTM anti-TNFα Fc fusion protein encoded by the transgene can include, but is not limited to, a therapeutic anti-TNFα TNFR2:Fc fusion that binds to TNFα, including but not limited to, etanercept. The amino acid sequences of the foregoing are provided in Table 6, infra. The TNFR2 domain having an amino acid sequence of SEQ ID NO: 2 and the Fc domain having an amino acid sequence of SEQ ID NO: 3 (SEQ ID NO 4 without the hinge region) and etanercept having the amino acid sequence of SEQ ID NO: 10 (SEQ ID NO: 11 including leader sequence) with a transgene nucleotide sequence of one of SEQ ID NOs: 13-18) The HuPTM anti-TNFα TNFR2:Fc fusion protein encoded by the transgene can include, but is not limited to, a dimeric fusion protein engineered to contain additional glycosylation sites on the Fc domain.

The compositions and methods provided herein systemically deliver the anti-TNFα Fc fusion protein, particularly, etanercept and EYS606, from a depot of viral genomes, for example, in the subject's eye, or liver/muscle, at a level either in the ocular tissue (e.g., in the vitreous or aqueous humor, or in the serum that is therapeutically or prophylactically effective to treat or ameliorate the symptoms of non-infectious uveitis or other indication that may be treated with an anti-TNFα Fc fusion protein. Identified herein are viral vectors for delivery of transgenes encoding the therapeutic anti-TNFα Fc fusion protein to cells in the human subject, including, in embodiments, one or more ocular tissue cells, and regulatory elements operably linked to the nucleotide sequence encoding the anti-TNFα Fc fusion protein that promote the expression of the antibody in the cells, in embodiments, in the ocular tissue cells. Such regulatory elements, including ocular tissue-specific regulatory elements, are provided in Table 1 and Table 1a herein. Accordingly, such viral vectors may be delivered to the human subject at appropriate dosages, such that at least 20, 30, 40, 50 or 60 days after administration, anti-TNFα Fc fusion protein is present at therapeutically effective levels in the serum or in ocular tissues of said human subject. In embodiments, the therapeutically effective level of the anti-anti-TNFα Fc fusion protein is determined (in human trials, animal models, such as those described herein, etc.) to improve best corrected visual acuity (BCVA) by >=2 ETDRS lines or increase in log MAR, reduced inflammatory activity of the anterior and posterior chamber according to the SUN classification, and/or reduction in grade of vitreous haze

The recombinant vector used for delivering the transgene includes non-replicating recombinant adeno-associated virus vectors (“rAAV”). rAAVs are particularly attractive vectors for a number of reasons—they can transduce non-replicating cells, and therefore, can be used to deliver the transgene to tissues where cell division occurs at low levels, such as the retina; they can be modified to preferentially target a specific organ of choice; and there are hundreds of capsid serotypes to choose from to obtain the desired tissue specificity, and/or to avoid neutralization by pre-existing patient antibodies to some AAVs. Such rAAVs include but are not limited to AAV based vectors comprising capsid components from one or more of AAV1, AAV2, AAV2.7m8 AAV3, AAV3B, AAV4, AAV5, AAV6, AAV7, AAV8, AAVrh8, AAV9, AAV9e, AAVrh10, AAVrh20, AAVrh39, AAVhu.37, AAVrh73, AAVrh74, AAV.hu51, AAV.hu21, AAV.hu12, or AAV.hu26. In certain embodiments, AAV based vectors provided herein comprise capsids from one or more ofAAV2.7m8, AAV8, AAV9, AAV10, or AAVrh10 serotypes.

However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements.

Gene therapy constructs for the therapeutic anti-TNFα Fc fusion protein are designed such that the covalently linked TNFR and the Fc portion are expressed as a dimeric fusion protein. For example, the TNFR:Fc fusion may include all or a portion of a hinge region (e.g. SEQ ID NO: 5 or 9) that is linked to disulfide bond formation between heavy chains. Preferably, TNFR1: Fc is a biobetter or a biosimilar of EYS606, or more preferably, TNFR: Fc is EYS606. Preferably, TNFR2: Fc is a biobetter or a biosimilar of etanercept, or more preferably, TNFR: Fc is etanercept.

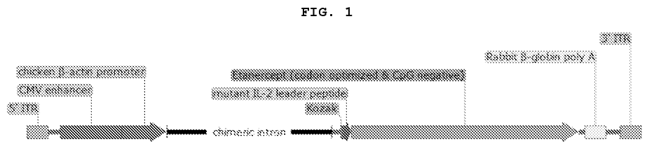

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161) and may also be optimized to reduce CpG dimers. Nucleotide sequences of the therapeutic anti-TNFα Fc fusion proteins, which may be codon optimized, are disclosed in Table 7. The anti-TNFα Fc fusion protein requires a N-terminal leader to ensure proper post-translation processing and secretion. Useful leader sequences for the expression of the therapeutic anti-TNFα Fc fusion protein in human cells are disclosed herein. An exemplary recombinant expression construct is shown in FIG. 1.

The production of HuPTM anti-TNFα Fc fusion proteins should result in a “biobetter” molecule for the treatment of disease accomplished via gene therapy—e.g., by administering a viral vector or other DNA expression construct encoding an anti-TNFα Fc fusion protein, such as etanercept or EYS606, to a patient (human subject) diagnosed with a disease indication for that anti-TNFα Fc fusion protein, to create a permanent depot in the subject that continuously supplies the human-glycosylated, sulfated transgene product produced by the subject's transduced cells. The cDNA construct for the HuPTM anti-TNFα Fc fusion protein should include a signal peptide that ensures proper co- and post-translational processing (glycosylation and protein sulfation) by the transduced human cells.

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil.

As an alternative, or an additional treatment to gene therapy, the full-length HuPTM anti-TNFα Fc fusion protein can be produced in human cell lines by recombinant DNA technology, and the glycoprotein can be administered to patients. Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. 36(6):1110-1122, which is incorporated by reference in its entirety for a review of the human cell lines that could be used for the recombinant production of the HuPTM biologic). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in human cells.

It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment of the invention is to slow or arrest the progression of disease.

Combination therapies involving delivery of the HuPTM anti-TNFα Fc fusion protein to the patient accompanied by administration of other available treatments are encompassed by the methods of the invention. The additional treatments may be administered before, concurrently or subsequent to the gene therapy treatment. Such additional treatments can include but are not limited to co-therapy with the therapeutic anti-TNFα Fc fusion protein.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic anti-TNFα Fc fusion protein, operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

### 5.1 Definitions

The phrase “Anti-TNFα Fc fusion protein” means a polypeptide comprising the soluble, extracellular domain or portion of a receptor for TNFα, including TNF-α receptor p55 or p75, covalently linked, for example, through a peptide bond, to an Fc domain of an immunoglobulin, particularly an IgG.

The term “regulatory element” or “nucleic acid regulatory element” are non-coding nucleic acid sequences that control the transcription of neighboring genes. Cis regulatory elements typically regulate gene transcription by binding to transcription factors. This includes “composite nucleic acid regulatory elements” comprising more than one enhancer or promoter elements as described herein.

The term “expression cassette” or “nucleic acid expression cassette” refers to nucleic acid molecules that include one or more transcriptional control elements including, but not limited to promoters, enhancers and/or regulatory elements, introns and polyadenylation sequences. The enhancers and promoters typically function to direct (trans)gene expression in one or more desired cell types, tissues or organs.

The term “operably linked” and “operably linked to” refers to nucleic acid sequences being linked and typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked and still be functional while not directly contiguous with a downstream promoter and transgene.

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into or modification of the amino acid sequence of the naturally-occurring capsid.

The term “rAAV” refers to a “recombinant AAV” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

The terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

The terms “subject”, “host”, and “patient” are used interchangeably. As used herein, a subject is preferably a mammal such as a non-primate (e.g., cows, pigs, horses, cats, dogs, rats etc.) or a primate (e.g., monkey and human), most preferably a human.

The terms “therapeutic agent” or “biotherapeutic agent” refer to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

The phrase “liver-specific” or “liver-directed” refers to nucleic acid elements that have adapted their activity in liver (hepatic) cells or tissue due to the interaction of such elements with the intracellular environment of the hepatic cells. The liver acts as a bioreactor or “depot” for the body in the context of a gene therapy delivered to the liver tissue and a gene cassette enhanced for expression in the liver will produce the biotherapeutic (translated protein) that is secreted into the circulation. As such, the biotherapeutic agent is delivered systemically to the subject by way of liver expression. Without being bound by any one theory, liver production of a biotherapeutic agent (such as produced by the delivered transgene) can provide immunotolerance to the agent such that endogenous T cells of the subject producing the protein will recognize the protein as self-protein, and not induce an innate immune response.

### 5.2 Constructs

Viral vectors or other DNA expression constructs encoding an HuPTM anti-TNFα Fc fusion protein are provided herein. The viral vectors and other DNA expression constructs provided herein include any suitable method for delivery of a transgene to a target cell. The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to ocular tissue cells or a vector that has a tropism for ocular tissue cells.

In some aspects, the disclosure provides for a nucleic acid for use, wherein the nucleic acid comprises a nucleotide sequence that encodes a HuPTM anti-TNFα Fc fusion protein, as a transgene described herein, operatively linked to a promoter selected for expression in tissue targeted for expression of the transgene, for example, but not limited to the ubiquitous promoters, such as CB7/CAG promoter (SEQ ID NO:50) and associated upstream regulatory sequences, cytomegalovirus (CMV) promoter, EF-1 alpha promoter (SEQ ID NO:53), mU1a (SEQ ID NO:52), UB6 promoter, chicken beta-actin (CBA) promoter, and ocular-tissue specific promoters, such as human rhodopsin kinase (GRK1) promoter (SEQ ID NOS:54 or 117), a mouse cone arresting (CAR) promoter (SEQ ID NOS:114-116), a human red opsin (RedO) promoter (SEQ ID NO:112), a CB promoter (SEQ ID NO: 159), a CBLong promoter (SEQ ID NO: 160), or a Best1/GRK promoter (SEQ ID NO: 161). See Tables 1 and 1a for a list of useful promoters.

In certain embodiments, provided herein are recombinant vectors that comprise one or more nucleic acids (e.g., polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., the nucleotide sequences encoding a HuPTM anti-TNFα Fc fusion protein), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161). Nucleotide sequences for expression of the anti-TNFα Fusion protein in human cells are provided herein in Table 7.

In a specific embodiment, the constructs described herein comprise the following components: (1) AAV2 inverted terminal repeats that flank the expression cassette; (2) one or more control elements, b) optionally, a chicken β-actin or other intron and c) a rabbit β-globin poly A signal; and (3) nucleic acid sequences coding for a soluble extracellular domain of TNFR2 linked to the N-terminus of the human IgG1 Fc domain, wherein the Fc domain comprises from the N-terminus to the C-terminus: Hinge region—CH2 domain-CH3 domain (etanercept). An exemplary construct is shown in FIG. 1 and in FIG. 2A.

In a specific embodiment, the constructs described herein comprise the following components: (1) AAV2 inverted terminal repeats that flank the expression cassette; (2) one or more control elements, b) optionally, a chicken β-actin or other intron and c) a rabbit β-globin poly A signal; and (3) nucleic acid sequences coding for a soluble extracellular domain of TNFR1 linked via a thrombin cleavage site to the N-terminus of the human IgG1 Fc domain, wherein the Fc domain comprises from the N-terminus to the C-terminus: Hinge region—CH2 domain-CH3 domain (EYS060). An exemplary construct is shown in FIG. 2B.

### 5.2.1 mRNA Vectors

In certain embodiments, as an alternative to DNA vectors, the vectors provided herein are modified mRNA encoding for the gene of interest (e.g., the transgene, for example, HuPTM non-monoclonal antibody biologic). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for a HuPTM anti-TNFα Fc fusion protein.

### 5.2.2 Viral Vectors

Viral vectors include adenovirus, adeno-associated virus (AAV, e.g., AAV2.7m8, AAV3, AAV8, AAV9, AAVrh10, AAV2.7m8, and other serotypes), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV) and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

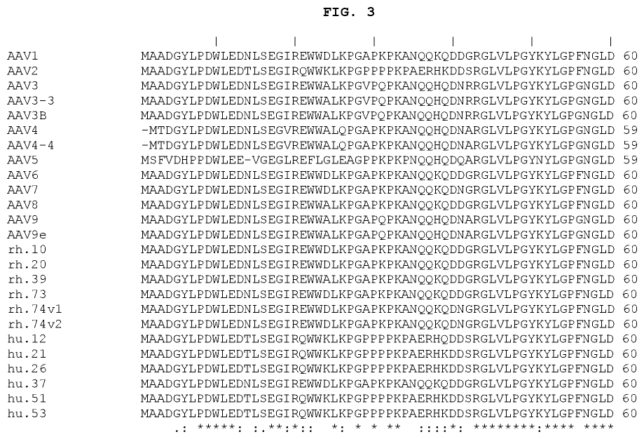

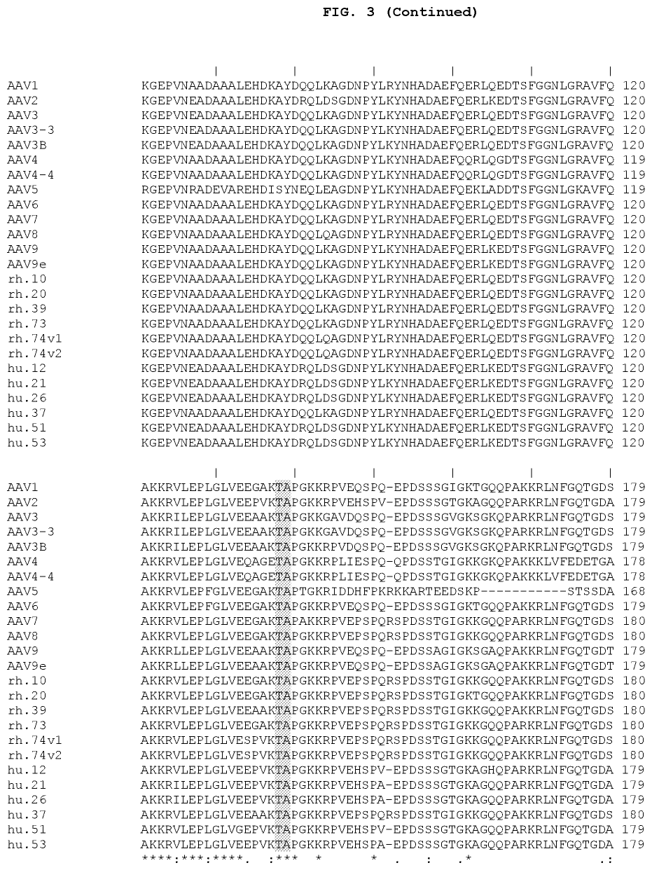

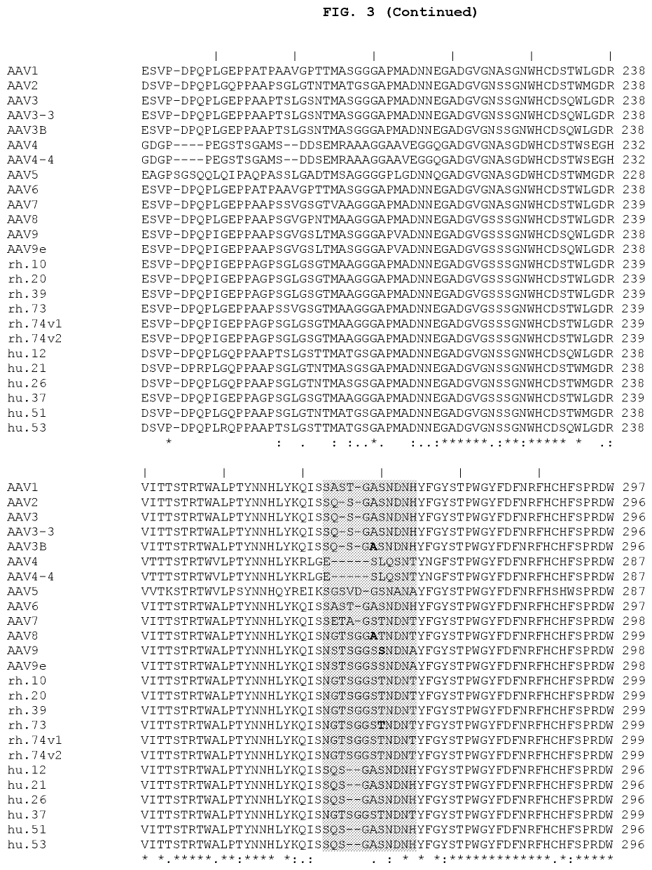

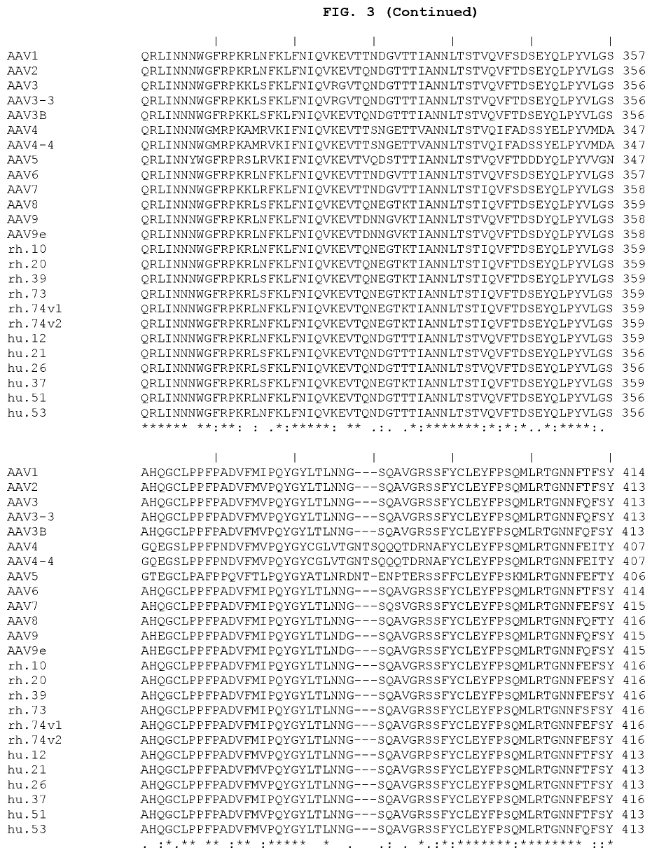

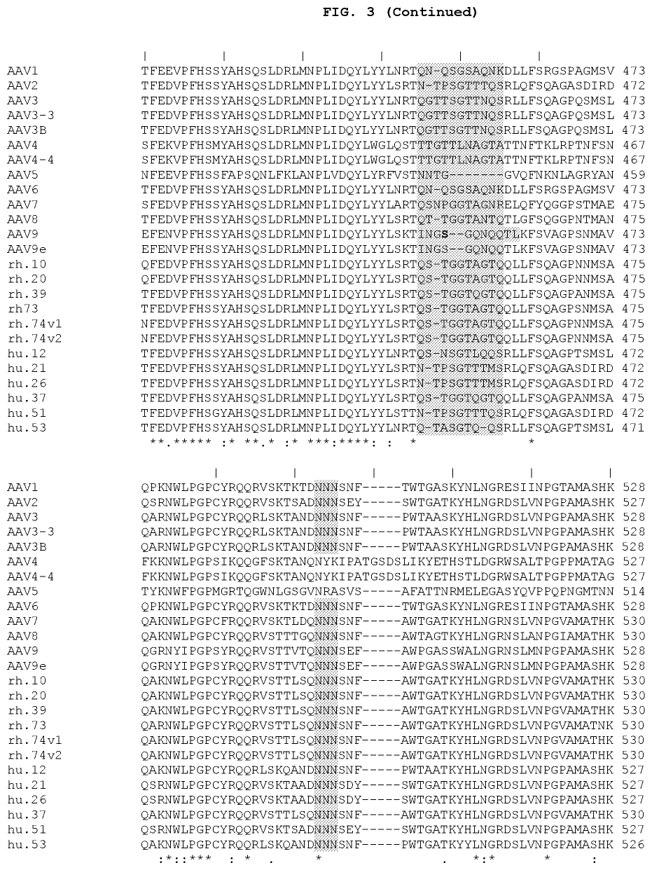

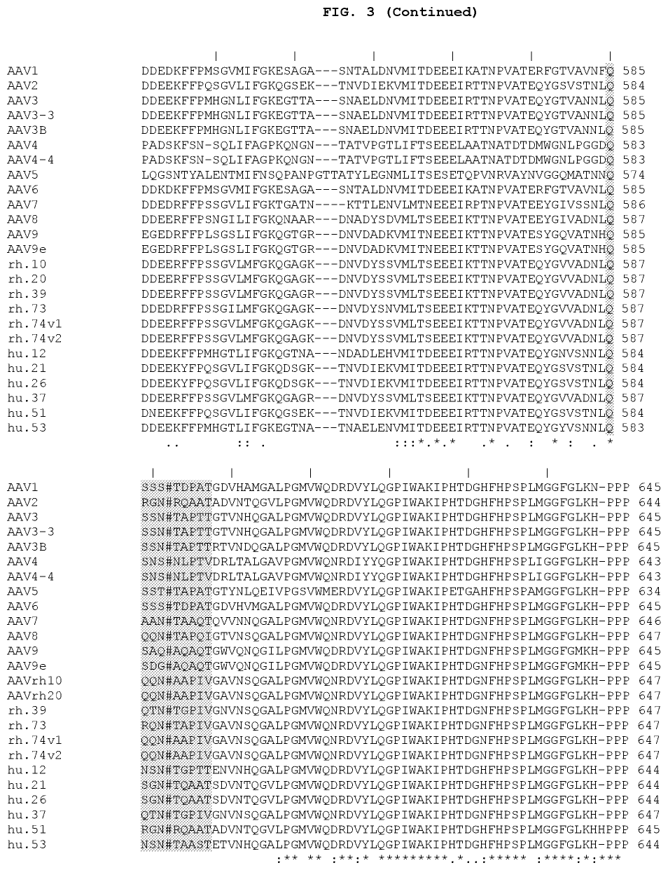

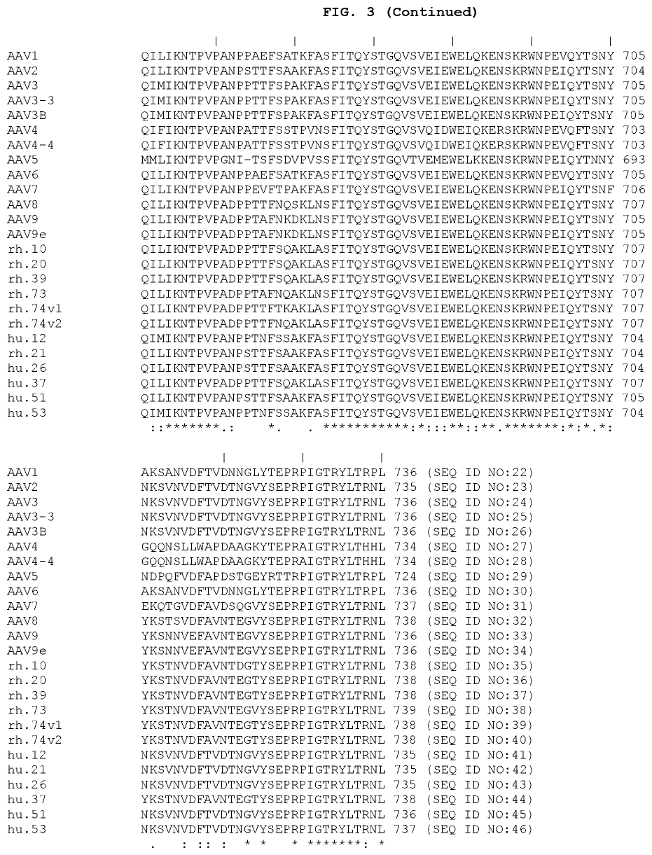

In certain embodiments, the viral vectors provided herein are AAV based viral vectors. In certain embodiments, the AAV-based vectors provided herein do not encode the AAV rep gene (required for replication) and/or the AAV cap gene (required for synthesis of the capsid proteins) (the rep and cap proteins may be provided by the packaging cells in trans). Multiple AAV serotypes have been identified. In certain embodiments, AAV-based vectors provided herein comprise components from one or more serotypes of AAV In preferred embodiments, AAV-based vectors provided herein comprise components from one or more serotypes of AAV with tropism to ocular tissues, liver and/or muscle. Throughout the specification, AAV “serotype” refers to an AAV having an immunologically distinct capsid, a naturally-occurring capsid, or an engineered capsid. In certain embodiments, AAV based vectors provided herein comprise capsid components from one or more of AAV1, AAV2, AAV2.7m8, AAV3, AAV3B, AAV4, AAV5, AAV6, AAV7, AAV8, AAVrh8, AAV9, AAV9e, AAVrh10, AAVrh20, AAVrh39, AAVhu.37, AAVrh73, AAVrh74, AAV.hu51, AAV.hu21, AAV.hu12, or AAV.hu26. In certain embodiments, AAV based vectors provided herein are or comprise components from one or more of AAV2.7m8, AAV8, AAV3B, AAV9, AAV10, AAVrh73, or AAVrh10 serotypes. Provided are viral vectors in which the capsid protein is a variant of the AAV8 capsid protein (SEQ ID NO:32), AAV3B capsid protein (SEQ ID NO:26), or AAVrh73 capsid protein (SEQ ID NO:38), and the capsid protein is e.g., at least 95%, 96%, 97%, 98%, 99% or 99.9% identical to the amino acid sequence of the AAV8 capsid protein (SEQ ID NO:32), AAV3B capsid protein (SEQ ID NO:26), or AAVrh73 capsid protein (SEQ ID NO:38), while retaining the biological function of the native capsid. In certain embodiments, the encoded AAV capsid has the sequence of SEQ ID NO:32 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8, AAV3B, or AAVrh73 capsid. FIG. 3 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV vector comprises an AAV8, AAV3B, or AAVrh73, capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions that are not present at that position in the native AAV capsid sequence as identified in the SUBS row of FIG. 3. Amino acid sequence for AAV8, AAV3B, or AAVrh73 capsids are provided in FIG. 3.

In some embodiments, AAV-based vectors comprise components from one or more serotypes of AAV. In some embodiments, AAV based vectors provided herein comprise capsid components from one or more ofAAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAVHSC12, AAV.HSC13, AAVHSC14, AAV.HSC15, or AAV.HSC16 or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAVHSC12, AAV.HSC13, AAVHSC14, AAV.HSC15, or AAV.HSC16 or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAVHSC2, AAVHSC3, AAVHSC4, AAV.HSC5, AAV.HSC6, AAVHSC7, AAVHSC8, AAV.HSC9, AAVHSC10, AAVHSC11, AAV.HSC12, AAVHSC13, AAVHSC14, AAV.HSC15, or AAV.HSC16, or a derivative, modification, or pseudotype thereof.

In particular embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In particular embodiments, the recombinant AAV for use in compositions and methods herein is AAV7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSCS, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

AAV2.7m8-based, AAV8-based, AAV9-based, and AAVrh10-based viral vectors are used in certain of the methods described herein. Nucleotide sequences of AAV based viral vectors and methods of making recombinant AAV and AAV capsids are taught, for example, in U.S. Pat. No. 9,193,956 B2, U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV2.7m8, AAV8, AAV9 or AAVrh10)-based viral vectors encoding a transgene (e.g., an HuPTM dimeric fusion protein). The amino acid sequences of AAV capsids, including AAV2.7m8, AAV8, AAV9 and AAVrh10 are provided in FIG. 3.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the transgene encoding the HuPTM anti-TNFα Fc fusion protein. The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the transgene encoding the HuPTM anti-TNFα Fc fusion protein. Four plasmids are used to make the construct: Gag/pol sequence containing plasmid, Rev sequence containing plasmids, Envelope protein containing plasmid (e.g., VSV-G), and Cis plasmid with the packaging elements and the anti-TNFα Fc fusion protein.

For lentiviral vector production, the four plasmids are co-transfected into cells (e.g., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an HuPTM anti-TNFα Fc fusion protein, such that, upon introduction of the vector into a relevant cell, a glycosylated and/or tyrosine sulfated variant of the HuPTM anti-TNFα Fc fusion protein is expressed by the cell.

### 5.2.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters that control expression of the transgene. These promoters (and other regulatory elements that control transcription, such as enhancers) may be constitutive (promote ubiquitous expression) or may specifically or selectively express in ocular tissues. In certain embodiments, the promoter is a constitutive promoter.

In certain embodiments, the promoter is a CB7 (also referred to as a CAG promoter) (see Dinculescu et al., 2005, Hum Gene Ther 16: 649-663, incorporated by reference herein in its entirety). In some embodiments, the CAG (SEQ ID NO:51) or CB7 promoter (SEQ ID NO:50) includes other expression control elements that enhance expression of the transgene driven by the vector. In certain embodiments, the other expression control elements include chicken 3-actin intron and/or rabbit β-globin polyA signal (SEQ ID NO:55). In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs.

In certain embodiments, the vectors provided herein comprise one or more tissue specific promoters (e.g., a retinal-specific promoter). In particular embodiments, the viral vectors provided herein comprises a ocular tissue cell specific promoter, such as, human rhodopsin kinase (GRK1) promoter (SEQ ID NOS:54 or 117), a mouse cone arresting (CAR) promoter (SEQ ID NOS:114-116), a human red opsin (RedO) promoter (SEQ ID NO:112) or a Best1/GRK promoter (SEQ ID NO: 161).

Provided are nucleic acid regulatory elements that are chimeric with respect to arrangements of elements in tandem in the expression cassette. Regulatory elements, in general, have multiple functions as recognition sites for transcription initiation or regulation, coordination with cell-specific machinery to drive expression upon signaling, and to enhance expression of the downstream gene.

In certain embodiments, the promoter is an inducible promoter. In certain embodiments the promoter is a hypoxia-inducible promoter. In certain embodiments, the promoter comprises a hypoxia-inducible factor (HIF) binding site. In certain embodiments, the promoter comprises a HIF-1a binding site. In certain embodiments, the promoter comprises a HIF-2a binding site. In certain embodiments, the HIF binding site comprises an RCGTG motif. For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety. In specific embodiments, the hypoxia-inducible promoter is the human N-WASP promoter, see, e.g., Salvi, 2017, Biochemistry and Biophysics Reports 9:13-21 (incorporated by reference for the teaching of the N-WASP promoter) or is the hypoxia-induced promoter of human Epo, see, e.g., Tsuchiya et al., 1993, J. Biochem. 113:395-400 (incorporated by reference for the disclosure of the Epo hypoxia-inducible promoter). In other embodiments, the promoter is a drug inducible promoter, for example, a promoter that is induced by administration of rapamycin or analogs thereof. See, e.g., the disclosure of rapamycin inducible promoters in PCT publications WO94/18317, WO 96/20951, WO 96/41865, WO 99/10508, WO 99/10510, WO 99/36553, and WO 99/41258, and U.S. Pat. No. 7,067,526, which are hereby incorporated by reference in their entireties for the disclosure of drug inducible promoters.

Provided herein are constructs containing certain ubiquitous and tissue-specific promoters. Such promoters include synthetic and tandem promoters. Examples and nucleotide sequences of promoters are provided in Tables 1 and 1a below. Table 1 also includes the nucleotide sequences of other regulatory elements useful for the expression cassettes provided herein.

TABLE 1
Promoter and Other Regulatory Element Sequences

| Name/SEQ ID NO. | Sequence |
|---|---|
| CAG/CB7 | gacattgattattgactagttattaatagtaatcaattacggggtcattagt |
| SEQ ID NO: 50 | tcatagcccatatatggagttccgcgttacataacttacggtaaatggcccg |
| | cctggctgaccgcccaacgacccccgcccattgacgtcaataatgacgtatg |
| | ttcccatagtaacgccaatagggactttccattgacgtcaatgggtggagta |
| | tttacggtaaactgcccacttggcagtacatcaagtgtatcatatgccaagt |
| | acgccccctattgacgtcaatgacggtaaatggcccgcctggcattatgccc |
| | agtacatgaccttatgggactttcctacttggcagtacatctacgtattagt |
| | catcgctattaccatggtcgaggtgagccccacgttctgcttcactctcccc |
| | atctcccccccctccccacccccaattttgtatttatttattttttaattat |
| | tttgtgcagcgatgggggcggggggggggggggggcgcgcgccaggcggggc |
| | ggggcggggcgaggggcggggcggggcgaggcggagaggtgcggcggcagcc |
| | aatcagagcggcgcgctccgaaagtttccttttatggcgaggcggcggcggc |
| | ggcggccctataaaaagcgaagcgcgcggcgggcgggagtcgctgcgcgctg |
| | ccttcgccccgtgccccgctccgccgccgcctcgcgccgcccgccccggctc |
| | tgactgaccgcgttactcccacaggtgagcgggcgggacggcccttctcctc |
| | cgggctgtaattagcgcttggtttaatgacggcttgtttcttttctgtggct |
| | gcgtgaaagccttgaggggctccgggagggccctttgtgcggggggagcggc |
| | tcggggggtgcgtgcgtgtgtgtgtgcgtggggagcgccgcgtgcggctccg |
| | cgctgcccggcggctgtgagcgctgcgggcgcggcgcggggctttgtgcgct |
| | ccgcagtgtgcgcgaggggagcgcggccgggggcggtgccccgcggtgcggg |
| | gggggctgcgaggggaacaaaggctgcgtgcggggtgtgtgcgtgggggggt |
| | gagcagggggtgtgggcgcgtcggtcgggctgcaaccccccctgcacccccc |
| | tccccgagttgctgagcacggcccggcttcgggtgcggggctccgtacgggg |
| | cgtggcgcggggctcgccgtgccgggcggggggtggcggcaggtgggggtgc |
| | cgggcggggcggggccgcctcgggccggggagggctcgggggaggggcgcgg |
| | cggcccccggagcgccggcggctgtcgaggcgcggcgagccgcagccattgc |
| | cttttatggtaatcgtgcgagagggcgcagggacttcctttgtcccaaatct |
| | gtgcggagccgaaatctgggaggcgccgccgcaccccctctagcgggcgcgg |
| | ggcgaagcggtgcggcgccggcaggaaggaaatgggcggggagggccttcgt |
| | gcgtcgccgcgccgccgtccccttctccctctccagcctcggggctgtccgc |
| | ggggggacggctgccttcgggggggacggggcagggcggggttcggcttctg |
| | gcgtgtgaccggcggctctagagcctctgctaaccatgttcatgccttcttc |
| | tttttcctacagctcctgggcaacgtgctggttattgtgctgtctcatcatt |
| | ttggcaaag |
| CAG (CMV- | gacattgattattgactagttattaatagtaatcaattacggggtcattagt |
| Chicken B-Actin | tcatagcccatatatggagttccgcgttacataacttacggtaaatggcccg |
| Promoter- | cctggctgaccgcccaacgacccccgcccattgacgtcaataatgacgtatg |
| chimeric intron) | ttcccatagtaacgccaatagggactttccattgacgtcaatgggtggagta |
| SEQ ID NO: 51 | tttacggtaaactgcccacttggcagtacatcaagtgtatcatatgccaagt |
| | acgccccctattgacgtcaatgacggtaaatggcccgcctggcattatgccc |
| | agtacatgaccttatgggactttcctacttggcagtacatctacgtattagt |
| | catcgctattaccatggtcgaggtgagccccacgttctgcttcactctcccc |
| | atctcccccccctccccacccccaattttgtatttatttattttttaattat |
| | tttgtgcagcgatgggggcggggggggggggggggcgcgcgccaggcggggc |
| | ggggcggggcgaggggcggggcggggcgaggcggagaggtgcggcggcagcc |
| | aatcagagcggcgcgctccgaaagtttccttttatggcgaggcggcggcggc |
| | ggcggccctataaaaagcgaagcgcgcggcgggcgggagtcgctgcgcgctg |
| | ccttcgccccgtgccccgctccgccgccgcctcgcgccgcccgccccggctc |
| | tgactgaccgcgttactcccacaggtgagcgggcgggacggcccttctcctc |
| | cgggctgtaattagcgcttggtttaatgacggcttgtttcttttctgtggct |
| | gcgtgaaagccttgaggggctccgggagggccctttgtgcggggggagcggc |
| | tcggggggtgcgtgcgtgtgtgtgtgcgtggggagcgccgcgtgcggctccg |
| | cgctgcccggcggctgtgagcgctgcgggcgcggcgcggggctttgtgcgct |
| | ccgcagtgtgcgcgaggggagcgcggccgggggcggtgccccgcggtgcggg |
| | gggggctgcgaggggaacaaaggctgcgtgcggggtgtgtgcgtgggggggt |
| | gagcagggggtgtgggcgcgtcggtcgggctgcaaccccccctgcacccccc |
| | tccccgagttgctgagcacggcccggcttcgggtgcggggctccgtacgggg |
| | cgtggcgcggggctcgccgtgccgggcggggggtggcggcaggtgggggtgc |
| | cgggcggggcggggccgcctcgggccggggagggctcgggggaggggcgcgg |
| | cggcccccggagcgccggcggctgtcgaggcgcggcgagccgcagccattgc |
| | cttttatggtaatcgtgcgagagggcgcagggacttcctttgtcccaaatct |
| | gtgcggagccgaaatctgggaggcgccgccgcaccccctctagcgggcgcgg |
| | ggcgaagcggtgcggcgccggcaggaaggaaatgggcggggagggccttcgt |
| | gcgtcgccgcgccgccgtccccttctccctctccagcctcggggctgtccgc |
| | ggggggacggctgccttcgggggggacggggcagggcggggttcggcttctg |
| | gcgtgtgaccggcggctctagagcctctgctaaccatgttcatgccttcttc |
| | tttttcctacag |
| mU1a | atggaggcggtactatgtagatgagaattcaggagcaaactgggaaaagcaa |
| SEQ ID NO: 52 | ctgcttccaaatatttgtgatttttacagtgtagttttggaaaaactcttag |
| | cctaccaattcttctaagtgttttaaaatgtgggagccagtacacatgaagt |
| | tatagagtgttttaatgaggcttaaatatttaccgtaactatgaaatgctac |
| | gcatatcatgctgttcaggctccgtggccacgcaactcatact |
| EF-1α (core) | gggcagagcgcacatcgcccacagtccccgagaagttggggggaggggtcgg |
| SEQ ID NO: 53 | caattgaacgggtgcctagagaaggtggcgcggggtaaactgggaaagtgat |
| | gtcgtgtactggctccgcctttttcccgagggtgggggagaaccgtatataa |
| | gtgcagtagtcgccgtgaacgttctttttcgcaacgggtttgccgccagaac |
| | acag |
| Human UbC | Gtctaacaaaaaagccaaaaacggccagaatttagcggacaatttactagtc |
| SEQ ID NO: 111 | taacactgaaAattacatattgacccaaatgattacatttcaaaaggtgcct |
| | aaaaaacttcacaaaacacactCgccaaccccgagcgcatagttcaaaaccg |
| | gagcttcagctacttaagaagataggtacataaAaccgaccaaagaaactga |
| | cgcctcacttatccctcccctcaccagaggtccggcgcctgtcgaTtcagga |
| | gagcctaccctaggcccgaaccctgcgtcctgcgacggagaaaagcctaccg |
| | cacaCctaccggcaggtggccccaccctgcattataagccaacagaacgggt |
| | gacgtcacgacacgaCgagggcgcgcgctcccaaaggtacgggtgcactgcc |
| | caacggcaccgccataactgccgcccCcgcaacagacgacaaaccgagttct |
| | ccagtcagtgacaaacttcacgtcagggtccccagatgGtgccccagcccat |
| | ctcacccgaataagagctttcccgcattagcgaaggcctcaagaccttgggT |
| | tcttgccgcccaccatgccccccaccttgtttcaacgacctcacagcccgcc |
| | tcacaagcgtcttcCattcaagactcgggaacagccgccattttgctgcgct |
| | ccccccaacccccagttcagggcaaccTtgctcgcggacccagactacagcc |
| | cttggcggtctctccacacgcttccgtcccaccgagcggccCggcggccacg |
| | aaagccccggccagcccagcagcccgctactcaccaagtgacgatcacagcg |
| | aTccacaaacaagaaccgcgacccaaatcccggctgcgacggaactagctgt |
| | gccacacccggcgCgtccttatataatcatcggcgttcaccgccccacggag |
| | atccctccgcagaatcgccgagaagggActacttttcctcgcctgttccgct |
| | ctctggaaagaaaaccagtgccctagagtcacccaagtcccgtCctaaaatg |
| | tccttctgctgatactggggttctaaggccgagtcttatgagcagcgggccg |
| | ctgtcctGagcgtccgggcggaaggatcaggacgctcgctgcgcccttcgtc |
| | tgacgtggcagcgctcgccgtgaggaggggggcgcccgcgggaggcgccaaa |
| | acccggcgcggaggcc |
| Human red opsin | ggaggctgaggggtggggaaagggcatgggtgtttcatgaggacagagcttc |
| (RedO) | cgtttcatgcaatgaaaagagtttggagacggatggtggtgactggactata |
| (photoreceptor- | cacttacacacggtagcgatggtacactttgtattatgtatattttaccacg |
| specific) | atctttttaaagtgtcaaaggcaaatggccaaatggttccttgtcctatagc |
| SEQ ID NO: 112 | tgtagcagccatcggctgttagtgacaaagcccctgagtcaagatgacagca |
| | gcccccataactcctaatcggctctcccgcgtggagtcatttaggagtagtc |
| | gcattagagacaagtccaacatctaatcttccaccctggccagggccccagc |
| | tggcagcgagggtgggagactccgggcagagcagagggcgctgacattgggg |
| | cccggcctggcttgggtccctctggcctttccccaggggccctctttccttg |
| | gggctttcttgggccgccactgctcccgctcctctccccccatcccaccccc |
| | tcaccccctcgttcttcatatccttctctagtgctccctccactttcatcca |
| | cccttctgcaagagtgtgggaccacaaatgagttttcacctggcctggggac |
| | acacgtgcccccacaggtgctgagtgactttctaggacagtaatctgcttta |
| | ggctaaaatgggacttgatcttctgttagccctaatcatcaattagcagagc |
| | cggtgaaggtgcagaacctaccgcctttccaggcctcctcccacctctgcca |
| | cctccactctccttcctgggatgtgggggctggcacacgtgtggcccagggc |
| | attggtgggattgcactgagctgggtcattagcgtaatcctggacaagggca |
| | gacagggcgagcggagggccagctccggggctcaggcaaggctgggggcttc |
| | ccccagacaccccactcctcctctgctggacccccacttcatagggcacttc |
| | gtgttctcaaagggcttccaaatagcatggtggccttggatgcccagggaag |
| | cctcagagttgcttatctccctctagacagaaggggaatctcggtcaagagg |
| | gagaggtcgccctgttcaaggccacccagccagctcatggcggtaatgggac |
| | aaggctggccagccatcccaccctcagaagggacccggtggggcaggtgatc |
| | tcagaggaggctcacttctgggtctcacattcttggatccggttccaggcct |
| | cggccctaaatagtctccctgggctttcaagagaaccacatgagaaaggagg |
| | attcgggctctgagcagtttcaccacccaccccccagtctgcaaatcctgac |
| | ccgtgggtccacctgccccaaaggcggacgcaggacagtagaagggaacaga |
| | gaacacataaacacagagagggccacagcggctcccacagtcaccgccacct |
| | tcctggcggggatgggtggggcgtctgagtttggttcccagcaaatccctct |
| | gagccgcccttgcgggctcgcctcaggagcaggggagcaagaggtgggagga |
| | ggaggtctaagtcccaggcccaattaagagatcaggtagtgtagggtttggg |
| | agcttttaaggtgaagaggcccgggctgatcccacaggccagtataaagcgc |
| | cgtgaccctcaggtgatgcgccagggccggctgccgtcggggacagggcttt |
| | ccatagcc |
| Human | Agatcttcccc a cctagccacc tggcaaactg ctccttctct |
| rhodopsin (Rho) | caaaggccca aacatggcct cccagactgc aacccccagg |
| (photoreceptor- | cagtcaggcc ctgtctccac aacctcacag ccaccctgga |
| specific) | cggaatctgc ttcttcccac atttgagtcc tcctcagccc |
| SEQ ID NO: 113 | ctgagctcct ctgggcaggg ctgtttcttt ccatctttgt |
| | attcccaggg gcctgcaaat aaatgtttaa tgaacgaaca |
| | agagagtgaa ttccaattcc atgcaacaag gattgggctc |
| | ctgggcccta ggctatgtgt ctggcaccag aaacggaagc |
| | tgcaggttgc agcccctgcc ctcatggagc tcctcctgtc |
| | agaggagtgt ggggactgga tgactccaga ggtaacttgt |
| | gggggaacga acaggtaagg ggctgtgtga cgagatgaga |
| | gactgggaga ataaaccaga aagtctctag ctgtccagag |
| | gacatagcac agaggcccat ggtccctatt tcaaacccag |
| | gccaccagac tgagctggga ccttgggaca gacaagtcat |
| | gcagaagtta ggggaccttc tcctcccttt tcctggatgg |
| | atcctgagta ccttctcctc cctgacctca ggcttcctcc |
| | tagtgtcacc ttggcccctc ttagaagcca attaggccct |
| | cagtttctgc agcggggatt aatatgatta tgaacacccc |
| | caatctccca gatgctgatt cagccaggag cttaggaggg |
| | 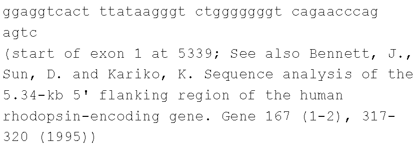 |
| Mouse cone | TGGCCCTGGCATTCCCCTATACTGGGACATAGAACCTTCACAGGACCAAGGG |
| arrestin promoter | CCTCTCCTCCCATTGATGACTGACTAGGCCATCCTCTAGCTACATAGGTGGT |
| (CAR) | GGAGCCTAGAGTCCCTCCTTGTGTACTCTTTGGTGGTGGTTACTCTGGGGGT |
| (photoreceptor- | ACTAGGTTAGTTCGTATTGTTGTTCCTCCTAGGGGACTGCAAACCCCTTCAG |
| specific) | CTCCTTGGGTCCTTTCTCTAGTTCCTTCTTTGGGGACCCTGTGCTCAGTTCA |
| SEQ ID NO: 114 | ATGGATGGCCAATTTCCTTCTTAAATGCCCCTAGCAGTAACTGTTAGGTCTC |
| | AATCCCAAGACAAATGTCTGAGGTGCCTATTTAACAGATCAAAGCGGACCTG |
| | TCCTCAGGTTAACCCAGTCACTCCCTGTACCTCAGTCCCTACCCATCACAAT |
| | TCTCCAGCCCATGAGCTTCGGGCTGTACTTCCCCAACGGGTTCTCCCATTTT |
| | GGGTACATGGCCTTTTTTTTTTACCTTTTTGGTTCCTTTGGCCTTTTGGCTT |
| | TTGGCTTCCAGGGCTTCTGGATCCCCCCCAACCCCTCCCATACACA |
| | TACACATGTGCACTCGTGCACTCAACCCAGCACAGGATAATGTTCATTCTTG |
| | ACCTTTCCACATACATCTGGCTATGTTCTCTCTCTTATCTACAATAAATCTC |
| | CTCCACTATACTTAGGAGCAGTTATGTTCTTCTTCTTTCTTTCTTTTTTTTT |
| | TTTTTTTCATTCAGTAACATCATCAGAATCCCCTAGCTCTGGCCTACCTCCT |
| | CAGTAACAATCAGCTGATCCCTGGCCACTAATCTGTACTCACTAATCTGTTT |
| | TCCAAACTCTTGGCCCCTGAGCTAATTATAGCAGTGCTTCATGCCACCCACC |
| | CCAACCCTATCCTTGTTCTCTGACTCCCACTAATCTACACATTCAGAGGATT |
| | GTGGATATAAGAGGCTGGGAGGCCAGCTTAGCAACCAGAGCTCGAGGCTGAT |
| | GCGAGCTTCATCTCTTCCCTC |
| Mouse cone | TTTTTTTTTTACCTTTTTGGTTCCTTTGGCCTTTTGGCTTTTGGCTTCCAGG |
| arrestin promoter | GCTTCTGGATCCCCCCCAACCCCTCCCATACACATACACATGTGCACTCGTG |
| (CAR) | CACTCAACCCAGCACAGGATA |
| (photoreceptor- | ATGTTCATTCTTGACCTTTCCACATACATCTGGCTATGTTCTCTCTCTTATC |
| specific) | TACAATAAATCTCCTCCACTATACTTAGGAGCAGTTATGTTCTTCTTCTTTC |
| SEQ ID NO: 115 | TTTCTTTTTTTTTTTTTTTTCATTCAGTAACATCATCAGAATCCCCTAGCTC |
| | TGGCCTACCTCCTCAGTAACAATCAGCTGATCCCTGGCCACTAATCTGTACT |
| | CACTAATCTGTTTTCCAAACTCTTGGCCCCTGAGCTAATTATAGCAGTGCTT |
| | CATGCCACCCACCCCAACCCTATCCTTGTTCTCTGACTCCCACTAATCTACA |
| | CATTCAGAGGATTGTGGATATAAGAGGCTGGGAGGCCAGCTTAGCAACCAGA |
| | GCTCGAGGCTGATGCGAGCTTCATCTCTTCCCTC |
| Mouse cone | TGATCCCTGGCCACTAATCTGTACTCACTAATCTGTTTTCCAAACTCTTGGC |
| arrestin promoter | CCCTGAGCTAATTATAGCAGTGCTTCATGCCACCCACCCCAACCCTATCCTT |
| (CAR) | GTTCTCTGACTCCCACTAATCTACACATTCAGAGGATTGTGGATATAAGAGG |
| (photoreceptor- | CTGGGAGGCCAGCTTAGCAACCAGAGCTCGAGGCTGATGCGAGCTTCATCTC |
| specific) | TTCCCTC |
| SEQ ID NO: 116 | |
| Human rhodopsin kinase (RK) or GRK1 (photoreceptor- specific) SEQ ID NO: 117 | 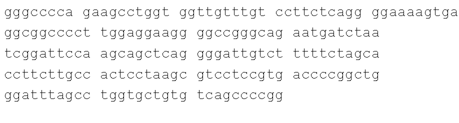 |
| Human | AAGG ACTCCTTTGT GGAGGTCCTG GCTTAGGGAG TCAAGTGACG |
| Bestrophin 1 | GCGGCTCAGC ACTCACGTGG GCAGTGCCAG CCTCTAAGAG |
| (BEST1) | TGGGCAGGGG CACTGGCCAC AGAGTCCCAG GGAGTCCCAC |
| promoter | CAGCCTAGTC GCCAGACC +38 |
| (a.k.a. hVMD2 | |
| promoter) | |
| SEQ ID NO: 118 | |
| Human | GTCAAGTGACG GCGGCTCAGC ACTCACGTGG GCAGTGCCAG |
| Bestrophin 1 | CCTCTAAGAG TGGGCAGGGG CACTGGCCAC AGAGTCCCAG |
| (BEST1) | GGAGTCCCAC CAGCCTAGTC GCCAGACC |
| promoter | (transcriptional start site residue underlined |
| (a.k.a. hVMD2 | at +1) |
| promoter) | |
| SEQ ID NO: 119 | |
| Human | CTCAGC ACTCACGTGG GCAGTGCCAG CCTCTAAGAG TGGGCAGGGG |
| Bestrophin 1 | CACTGGCCAC AGAGTCCCAG GGAGTCCCAC CAGCCTAGTC |
| (BEST1) | GCCAGAC |
| promoter | (transcriptional start site residue underlined |
| (a.k.a. hVMD2 | at +1) |
| promoter) | |
| SEQ ID NO: 120 | |
| Human | TTAATAAACA TTTGGGCGAT TCTTACGGCC TCTAAAGACC |
| Bestrophin 1 | AAGAACCACT GCTGCCTAGA GCTCTGCTCT CTTCATTGAA |
| (BEST1) | CAATACAAGA GGAGTGTGTA GGTAGACACC CACCACTTCC |
| promoter | AACAGCTTAG GAGAGCCCTT GAGTATGGAT TGATGTATTA |
| (a.k.a. hVMD2 | AAATTTATTG AATCACATGC TGAGATTTTC ACCAGCTGCC |
| promoter) | CGTGGGGATC TGGGCATTTA TTCCCATATT GCACTGGCTG |
| SEQ ID NO: 121 | GCTGGAAGCC AGCAGCATAA ACTCCAGGGC TGTTCTGTCA |
| | ACCCCCACCA GACTCACCCC GCTCCACCAG CCCCGGCAGG |
| | CTTCTCCTTC CATCTCTCTG AAGCAACTTA CTGATGGGCC |
| | CTGCCAGCCA ATCACAGCCA GAATAACGTA TGATGTCACC |
| | AGCAGCCAAT CAGAGCTCCT CGTCAGCATA TGCAGAATTC |
| | TGTCATTTTA CTAGGGTGAT GAAATTCCCA AGCAACACCA |
| | TCCTTTTCAG ATAAGGGCAC TGAGGCTGAG AGAGGAGCTG |
| | AAACCTACCC GGCGTCACCA CACACAGGTG GCAAGGCTGG |
| | GACCAGAAAC CAGGACTGTT GACTGCAGCC CGGTATTCAT |
| | TCTTTCCATA GCCCACAGGG CTGTCAAAGA CCCCAGGGCC |
| | TAGTCAGAGG CTCCTCCTTC CTGGAGAGTT CCTGGCACAG |
| | AAGTTGAAGC TCAGCACAGC CCCCTAACCC CCAACTCTCT |
| | CTGCAAGGCC TCAGGGGTCA GAACACTGGT GGAGCAGATC |
| | CTTTAGCCTC TGGATTTTAG GGCCATGGTA GAGGGGGTGT |
| | TGCCCTAAAT TCCAGCCCTG GTCTCAGCCC AACACCCTCC |
| | AAGAAGAAAT TAGAGGGGCC ATGGCCAGGC TGTGCTAGCC |
| | GTTGCTTCTG AGCAGATTAC AAGAAGGGAC CAAGACAAGG |
| | ACTCCTTTGT GGAGGTCCTG GCTTAGGGAG TCAAGTGACG |
| | GCGGCTCAGC ACTCACGTGG GCAGTGCCAG CCTCTAAGAG |
| | TGGGCAGGGG CACTGGCCAC AGAGTCCCAG GGAGTCCCAC |
| | CAGCCTAGTC GCCAGACCTT CTGTGGGATC ATCGGACCCA |
| | (transcriptional start site residue underlined |
| | at +1; minimal active promoter region in bold) |
| GRK1 promoter | GGGCCCCAGAAGCCTGGTGGTTGTTTGTCCTTCTCAGGGGAAAAGTGAGGCG |
| SEQ ID NO: 54 | GCCCCTTGGAGGAAGGGGCCGGGCAGAATGATCTAATCGGATTCCAAGCAGC |
| | TCAGGGGATTGTCTTTTTCTAGCACCTTCTTGCCACTCCTAAGCGTCCTCCG |
| | TGACCCCGGCTGGGATTTAGCCTGGTGCTGTGTCAGCCCCGGGCTCCCAGGG |
| | GCTTCCCAGTGGTCCCCAGGAACCCTCGACAGGGCCAGGGCGTCTCTCTCGT |
| | CCAGCAAGGGCAGGGACGGGCCACAGGCCAAGGGC |
| Upstream Kozak | GCCACC |
| sequence | |
| Rabbit beta- | gatctgacttctatctttttccctctgccaaaaattatggggacatcatgaa |
| globin polyA | gccccttgagcggctaataaaggaaatttattttcattgcaatagtgtgttg |
| SEQ ID NO: 55 | gaattttttgtgtctctcactcg |
| Chimeric Intron | gtaagtatcaaggttacaagacaggtttaaggagaccaatagaaactgggct |
| SEQ ID NO: 56 | tgtcgagacagagaagactcttgcgtttctgataggcacctattggtcttac |
| | tgacatccactttgcctttctctccacag |
| VH4 Intron | gtgagtatctcagggatccagacatggggatatgggaggtgcctctgatccc |
| SEQ ID NO: 57 | agggctcactgtgggtctctctgttcacag |
| SV40 Intron | gtaagtttagtctttttgtcttttatttcaggtcccggatccggtggtggtg |
| SEQ ID NO: 157 | caaatcaaagaactgctcctcagtggatgttgcctttacttctag |
| 5'ITR | ctgcgcgctcgctcgctcactgaggccgcccgggcaaagcccgggcgtcggg |
| SEQ ID NO: 58 | cgacctttggtcgcccggcctcagtgagcgagcgagcgcgcagagagggagt |
| | ggccaactccatcactaggggttcct |
| 5'-ITR | ctgcgcgctcgctcgctcactgaggccgcccgggcaaagcccgggcgtcggg |
| (Deleted D- | cgacctttggtcgcccggcctcagtgagcgagcgagcgcgcagagagggagt |
| sequence for | gg |
| self- | |
| complimentary | |
| AAV) | |
| SEQ ID NO: 59 | |
| 3'-ITR AAV | gaacccctagtgatggagttggccactccctctctgcgcgctcgctcgctca |
| SEQ ID NO: 60 | ctgaggccgcccgggcaaagcccgggcgtcgggcgacctttggtcgcccggc |
| | ctcagtgagcgagcgagcgcgcagagagggagtggccaa |
| 3'-ITR | ttggccactccctctctgcgcgctcgctcgctcactgaggccgggcgaccaa |
| (Deleted D- | aggtcgcccgacgcccgggctttgcccgggggcctcagtgagcgagcgagcg |
| sequence for | cgcag |
| self- | |
| complimentary | |
| AAV) | |
| SEQ ID NO: 61 | |
| CB promoter | gacgacattgattattgactagttattaatagtaatcaattacggggtcatt |
| SEQ ID NO: 159 | agttcatagcccatatatggagttccgcgttacataacttacggtaaatggc |
| | ccgcctggctgaccgcccaacgacccccgcccattgacgtcaataatgacgt |
| | atgttcccatagtaacgccaatagggactttccattgacgtcaatgggtgga |
| | gtatttacggtaaactgcccacttggcagtacatcaagtgtatcatatgcca |
| | agtacgccccctattgacgtcaatgacggtaaatggcccgcctggcattatg |
| | cccagtacatgaccttatgggactttcctacttggcagtacatctacgtatt |
| | agtcatcgctattaccatggtcgaggtgagccccacgttctgcttcactctc |
| | cccatctcccccccctccccacccccaattttgtatttatttattttttaat |
| | tattttgtgcagcgatgggggcggggggggggggggggcgcgcgccaggcgg |
| | ggcggggcggggcgaggggcggggcggggcgaggcggagaggtgcggcggca |
| | gccaatcagagcggcgcgctccgaaagtttccttttatggcgaggcggcggc |
| | ggcggcggccctataaaaagcgaagcgcgcggcgggcgg |
| CB long | gacgacattgattattgactagttattaatagtaatcaattacggggtcatt |
| promoter | agttcatagcccatatatggagttccgcgttacataacttacggtaaatggc |
| (CB+100) | ccgcctggctgaccgcccaacgacccccgcccattgacgtcaataatgacgt |
| SEQ ID NO: 160 | atgttcccatagtaacgccaatagggactttccattgacgtcaatgggtgga |
| | gtatttacggtaaactgcccacttggcagtacatcaagtgtatcatatgcca |
| | agtacgccccctattgacgtcaatgacggtaaatggcccgcctggcattatg |
| | cccagtacatgaccttatgggactttcctacttggcagtacatctacgtatt |
| | agtcatcgctattaccatggtcgaggtgagccccacgttctgcttcactctc |
| | cccatctcccccccctccccacccccaattttgtatttatttattttttaat |
| | tattttgtgcagcgatgggggcggggggggggggggggcgcgcgccaggcgg |
| | ggcggggcggggcgaggggcggggcggggcgaggcggagaggtgcggcggca |
| | gccaatcagagcggcgcgctccgaaagtttccttttatggcgaggcggcggc |
| | ggcggcggccctataaaaagcgaagcgcgcggcgggcgggagtcgctgcgcg |
| | ctgccttcgccccgtgccccgctccgccgccgcctcgcgccgcccgccccgg |
| | ctctgactgaccgcgttactcccacaggtgagcg |
| Best1/GRK1 | GTCGACAAATTCTGTCATTTTACTAGGGTGATGAAATTCCCAAGCAACACCA |
| tandem promoter | TCCTTTTCAGATAAGGGCACTGAGGCTGAGAGAGGAGCTGAAACCTACCCGG |
| SEQ ID NO:161 | GGTCACCACACACAGGTGGCAAGGCTGGGACCAGAAACCAGGACTGTTGACT |
| | GCAGCCCGGTATTCATTCTTTCCATAGCCCACAGGGCTGTCAAAGACCCCAG |
| | GGCCTAGTCAGAGGCTCCTCCTTCCTGGAGAGTTCCTGGCACAGAAGTTGAA |
| | GCTCAGCACAGCCCCCTAACCCCCAACTCTCTCTGCAAGGCCTCAGGGGTCA |
| | GAACACTGGTGGAGCAGATCCTTTAGCCTCTGGATTTTAGGGCCATGGTAGA |
| | GGGGGTGTTGCCCTAAATTCCAGCCCTGGTCTCAGCCCAACACCCTCCAAGA |
| | AGAAATTAGAGGGGCCATGGCCAGGCTGTGCTAGCCGTTGCTTCTGAGCAGA |
| | TTACAAGAAGGGACTAAGACAAGGACTCCTTTGTGGAGGTCCTGGCTTAGGG |
| | AGTCAAGTGACGGCGGCTCAGCACTCACGTGGGCAGTGCCAGCCTCTAAGAG |
| | TGGGCAGGGGCACTGGCCACAGAGTCCCAGGGAGTCCCACCAGCCTAGTCGC |
| | CAGAtCTAGAGGGCCCCAGAAGCCTGGTGGTTGTTTGTCCTTCTCAGGGGAA |
| | AAGTGAGGCGGCCCCTTGGAGGAAGGGGCCGGGCAGAAGATCTAATCGGATT |
| | CCAAGCAGCTCAGGGGATTGTCTTTTTCTAGCACCTTCTTGCCACTCCTAAG |
| | CGTCCTCCGTGACCCCGGCTGGGATTTAGCCTGGTGCTGTGTCAGCCCCGGG |
| | CTCCCAGGGGCTTCCCAGTGGTCCCCAGGAACCCTCGACAGGGCCAGGGCGT |
| | CTCTCTCGTCCAGCAAGGGCAGGGACGGGCCACAGGCCAAGGGCCTTAAGC |

TABLE 1a
Other regulatory sequences

| | |
|---|---|
| LSPX1 | aggttaatttttaaaaagcagtcaaaagtccaagtggcccttggcagcatttactctctctgt |
| SEQ ID NO: 122 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgtctagaaggctc |
| | agaggcacacaggagtttctgggctcaccctgcccccttccaacccctcagttcccatcctcc |
| | agcagctgtttgtgtgctgcctctgaagtccacactgaacaaacttcagcctactcatgtccc |
| | taaaatgggcaaacattgcaagcagcaaacagcaaacacacagccctccctgcctgctgacct |
| | tggagctggggcagaggtcagagacctctctgggcccatgccacctccaacatccactcgacc |
| | ccttggaatttcggtggagaggagcagaggttgtcctggcgtggtttaggtagtgtgagaggg |
| | gtacccggggatcttgctaccagtggaacagccactaaggattctgcagtgagagcagagggc |
| | cagctaagtggtactctcccagagactgtctgactcacgccaccccctccaccttggacacag |
| | gacgctgtggtttctgagccaggtacaatgactcctttcggtaagtgcagtggaagctgtaca |
| | ctgcccaggcaaagcgtccgggcagcgtaggcgggcgactcagatcccagccagtggacttag |
| | cccctgtttgctcctccgataactggggtgaccttggttaatattcaccagcagcctcccccg |
| | ttgcccctctggatccactgcttaaatacggacgaggacagggccctgtctcctcagcttcag |
| | gcaccaccactgacctgggacagt |
| LSXP2 | aggctcagaggcacacaggagtttctgggctcaccctgcccccttccaaccc |
| SEQ ID NO: 123 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggtctagaaggctcagaggcacacaggagtttctgggctcaccct |
| | gcccccttccaacccctcagttcccatcctccagcagctgtttgtgtgctgc |
| | ctctgaagtccacactgaacaaacttcagcctactcatgtccctaaaatggg |
| | caaacattgcaagcagcaaacagcaaacacacagccctccctgcctgctgac |
| | cttggagctggggcagaggtcagagacctctctgggcccatgccacctccaa |
| | catccactcgaccccttggaatttcggtggagaggagcagaggttgtcctgg |
| | cgtggtttaggtagtgtgagaggggtacccggggatcttgctaccagtggaa |
| | cagccactaaggattctgcagtgagagcagagggccagctaagtggtactct |
| | cccagagactgtctgactcacgccaccccctccaccttggacacaggacgct |
| | gtggtttctgagccaggtacaatgactcctttcggtaagtgcagtggaagct |
| | gtacactgcccaggcaaagcgtccgggcagcgtaggcgggcgactcagatcc |
| | cagccagtggacttagcccctgtttgctcctccgataactggggtgaccttg |
| | gttaatattcaccagcagcctcccccgttgcccctctggatccactgcttaa |
| | atacggacgaggacagggccctgtctcctcagcttcaggcaccaccactgac |
| | ctgggacagt |
| LTP1 | aggttaatttttaaaaagcagtcaaaagtccaagtggcccttggcagcattt |
| SEQ ID NO: 124 | actctctctgtttgctctggttaataatctcaggagcacaaacattccagat |
| | ccaggttaatttttaaaaagcagtcaaaagtccaagtggcccttggcagcat |
| | ttactctctctgtttgctctggttaataatctcaggagcacaaacattccag |
| | atccggcgcgccagggctggaagctacctttgacatcatttcctctgcgaat |
| | gcatgtataatttctacagaacctattagaaaggatcacccagcctctgctt |
| | ttgtacaactttcccttaaaaaactgccaattccactgctgtttggcccaat |
| | agtgagaactttttcctgctgcctcttggtgcttttgcctatggcccctatt |
| | ctgcctgctgaagacactcttgccagcatggacttaaacccctccagctctg |
| | acaatcctctttctcttttgttttacatgaagggtctggcagccaaagcaat |
| | cactcaaagttcaaaccttatcattttttgctttgttcctcttggccttggt |
| | tttgtacatcagctttgaaaataccatcccagggttaatgctggggttaatt |
| | tataactaagagtgctctagttttgcaatacaggacatgctataaaaatgga |
| | aagatgttgctttctgagaggatcttgctaccagtggaacagccactaagga |
| | ttctgcagtgagagcagagggccagctaagtggtactctcccagagactgtc |
| | tgactcacgccaccccctccaccttggacacaggacgctgtggtttctgagc |
| | caggtacagtgactcctttcggtaagtgcagtggaagctgtacactgcccag |
| | gcaaagcgtccgggcagcgtaggcgggcgactcagatcccagccagtggact |
| | tagcccctgtttgctcctccgataactggggtgaccttggttaatattcacc |
| | agcagcctcccccgttgcccctctggatccactgcttaaatacggacgagga |
| | cagggccctgtctcctcagcttcaggcaccaccactgacctgggacagt |
| LTP2 | aggctcagaggcacacaggagtttctgggctcaccctgcccccttccaaccc |
| SEQ ID NO: 125 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggtctagagcccttaagctagcaggttaatttttaaaaagcagtc |
| | aaaagtccaagtggcccttggcagcatttactctctctgtttgctctggtta |
| | ataatctcaggagcacaaacattccagatccaggttaatttttaaaaagcag |
| | tcaaaagtccaagtggcccttggcagcatttactctctctgtttgctctggt |
| | taataatctcaggagcacaaacattccagatccggcgcgccagggctggaag |
| | ctacctttgacatcatttcctctgcgaatgcatgtataatttctacagaacc |
| | tattagaaaggatcacccagcctctgcttttgtacaactttcccttaaaaaa |
| | ctgccaattccactgctgtttggcccaatagtgagaactttttcctgctgcc |
| | tcttggtgcttttgcctatggcccctattctgcctgctgaagacactcttgc |
| | cagcatggacttaaacccctccagctctgacaatcctctttctcttttgttt |
| | tacatgaagggtctggcagccaaagcaatcactcaaagttcaaaccttatca |
| | ttttttgctttgttcctcttggccttggttttgtacatcagctttgaaaata |
| | ccatcccagggttaatgctggggttaatttataactaagagtgctctagttt |
| | tgcaatacaggacatgctataaaaatggaaagatgttgctttctgagaggat |
| | cttgctaccagtggaacagccactaaggattctgcagtgagagcagagggcc |
| | agctaagtggtactctcccagagactgtctgactcacgccaccccctccacc |
| | ttggacacaggacgctgtggtttctgagccaggtacagtgactcctttcggt |
| | aagtgcagtggaagctgtacactgcccaggcaaagcgtccgggcagcgtagg |
| | cgggcgactcagatcccagccagtggacttagcccctgtttgctcctccgat |
| | aactggggtgaccttggttaatattcaccagcagcctcccccgttgcccctc |
| | tggatccactgcttaaatacggacgaggacagggccctgtctcctcagcttc |
| | aggcaccaccactgacctgggacagt |
| LTP3 | aggttaatttttaaaaagcagtcaaaagtccaagtggcccttggcagcattt |
| SEQ ID NO: 126 | actctctctgtttgctctggttaataatctcaggagcacaaacattccagat |
| | ccaggttaatttttaaaaagcagtcaaaagtccaagtggcccttggcagcat |
| | ttactctctctgtttgctctggttaataatctcaggagcacaaacattccag |
| | atccggcgcgccagggctggaagctacctttgacatcatttcctctgcgaat |
| | gcatgtataatttctacagaacctattagaaaggatcacccagcctctgctt |
| | ttgtacaactttcccttaaaaaactgccaattccactgctgtttggcccaat |
| | agtgagaactttttcctgctgcctcttggtgcttttgcctatggcccctatt |
| | ctgcctgctgaagacactcttgccagcatggacttaaacccctccagctctg |
| | acaatcctctttctcttttgttttacatgaagggtctggcagccaaagcaat |
| | cactcaaagttcaaaccttatcattttttgctttgttcctcttggccttggt |
| | tttgtacatcagctttgaaaataccatcccagggttaatgctggggttaatt |
| | tataactaagagtgctctagttttgcaatacaggacatgctataaaaatgga |
| | aagatgttgctttctgagaggatcttgctaccagtggaacagccactaagga |
| | ttctgcagtgagagcagagggccagctaagtggtactctcccagagactgtc |
| | tgactcacgccaccccctccaccttggacacaggacgctgtggtttctgagc |
| | caggtacagtgactcctttcggtaagtgcagtggaagctgtacactgcccag |
| | gcaaagcgtccgggcagcgtaggcgggcgactcagatcccagccagtggact |
| | tagcccctgtttgctcctccgataactggggtgaccttggttaatattcacc |
| | agcagcctcccccgttgcccctctggatccactgcttaaatacggacgagga |
| | cagggccctgtctcctcagcttcaggcaccaccactgacctgggacagtaaa |
| | acaggtaagtccgctgtttgtgtgctgcctctgaagtccacactgaacaaac |
| | ttcagcctactcatgtccctaaaatgggcaaacattgcaagcagcaaacagc |
| | aaacacacagccctccctgcctgctgaccttggagctggggcagaggtcaga |
| | gacctctctggcctctactaaccatgttcatgttttctttttttttctacag |
| | gtcctgggtgacgaacag |
| LMTP6 | aggctcagaggcacacaggagtttctgggctcaccctgcccccttccaaccc |
| SEQ ID NO: 127 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggccactacgggtttaggctgcccatgtaaggaggcaaggcctgg |
| | ggacacccgagatgcctggttataattaacccagacatgtggctgccccccc |
| | cccccccaacacctgctgcctctaaaaataaccctgtccctggtggatccca |
| | ctacgggtttaggctgcccatgtaaggaggcaaggcctggggacacccgaga |
| | tgcctggttataattaacccagacatgtggctgccccccccccccccaacac |
| | ctgctgcctctaaaaataaccctgtccctggtggatcccactacgggtttag |
| | gctgcccatgtaaggaggcaaggcctggggacacccgagatgcctggttata |
| | attaacccagacatgtggctgccccccccccccccaacacctgctgcctcta |
| | aaaataaccctgtccctggtggatcccctgcatgcgaagatcttcgaacaag |
| | gctgtgggggactgagggcaggctgtaacaggcttgggggccagggcttata |
| | cgtgcctgggactcccaaagtattactgttccatgttcccggcgaagggcca |
| | gctgtcccccgccagctagactcagcacttagtttaggaaccagtgagcaag |
| | tcagcccttggggcagcccatacaaggccatggggctgggcaagctgcacgc |
| | ctgggtccggggtgggcacggtgcccgggcaacgagctgaaagctcatctgc |
| | tctcaggggcccctccctggggacagcccctcctggctagtcacaccctgta |
| | ggctcctctatataacccaggggcacaggggctgccctcattctaccaccac |
| | ctccacagcacagacagacactcaggagccagccagcgtcgagatcttgcta |
| | ccagtggaacagccactaaggattctgcagtgagagcagagggccagctaag |
| | tggtactctcccagagactgtctgactcacgccaccccctccaccttggaca |
| | caggacgctgtggtttctgagccaggtacagtgactcctttcggtaagtgca |
| | gtggaagctgtacactgcccaggcaaagcgtccgggcagcgtaggcgggcga |
| | ctcagatcccagccagtggacttagcccctgtttgctcctccgataactggg |
| | gtgaccttggttaatattcaccagcagcctcccccgttgcccctctggatcc |
| | actgcttaaatacggacgaggacagggccctgtctcctcagcttcaggcacc |
| | accactgacctgggacagt |
| LMTP13 | aggctcagaggcacacaggagtttctgggctcaccctgcccccttccaaccc |
| SEQ ID NO: 128 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagaggggtacccggggatcttgctaccagtctagaggccgtccgccctc |
| | ggcaccatcctcacgacacccaaatatggcgacgggtgaggaatggtgggga |
| | gttatttttagagcggtgaggaaggtgggcaggcagcaggtgttggcgctct |
| | aaaaataactcccgggagttatttttagagcggaggaatggtggacacccaa |
| | atatggcgacggttcctcacccgtcgccatatttgggtgtccgccctcggcc |
| | ggggccgcattcctgggggccgggcggtgctcccgcccgcctcgataaaagg |
| | ctccggggccggcggcggcccacgagctacccggaggagcgggaggcgccaa |
| | gcgtgagtatcgatcttgctaccagtggaacagccactaaggattctgcagt |
| | gagagcagagggccagctaagtggtactctcccagagactgtctgactcacg |
| | ccaccccctccaccttggacacaggacgctgtggtttctgagccaggtacag |
| | tgactcctttcggtaagtgcagtggaagctgtacactgcccaggcaaagcgt |
| | ccgggcagcgtaggcgggcgactcagatcccagccagtggacttagcccctg |
| | tttgctcctccgataactggggtgaccttggttaatattcaccagcagcctc |
| | ccccgttgcccctctggatccactgcttaaatacggacgaggacagggccct |
| | gtctcctcagcttcaggcaccaccactgacctgggacagt |
| LMTP14 | gaatggtggacacccaaatatggcgacggttcctcacccgtcgccatatttg |
| SEQ ID NO: 129 | ggtgtccgccctcggccggggccgcattcctgggggccgggcggtgctcccg |
| | cccgcctcgataaaaggctccggggccggcggcggcccacgagctacccgga |
| | ggagcgggaggcgccaagcgatcttgctaccagtggaacagccactaaggat |
| | tctgcagtgagagcagagggccagctaagtggtactctcccagagactgtct |
| | gactcacgccaccccctccaccttggacacaggacgctgtggtttctgagcc |
| | aggtacagtgactcctttcggtaagtgcagtggaagctgtacactgcccagg |
| | caaagcgtccgggcagcgtaggcgggcgactcagatcccagccagtggactt |
| | agcccctgtttgctcctccgataactggggtgaccttggttaatattcacca |
| | gcagcctcccccgttgcccctctggatccactgcttaaatacggacgaggac |
| | agggccctgtctcctcagcttcaggcaccaccactgacctgggacagt |
| LMTP15 | aggctcagaggcacacaggagtttctgggctcaccctgcccccttccaaccc |
| SEQ ID NO: 130 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggtctagagaatggtggacacccaaatatggcgacggttcctcac |
| | ccgtcgccatatttgggtgtccgccctcggccggggccgcattcctgggggc |
| | cgggcggtgctcccgcccgcctcgataaaaggctccggggccggcggcggcc |
| | cacgagctacccggaggagcgggaggcgccaagcgatcttgctaccagtgga |
| | acagccactaaggattctgcagtgagagcagagggccagctaagtggtactc |
| | tcccagagactgtctgactcacgccaccccctccaccttggacacaggacgc |
| | tgtggtttctgagccaggtacagtgactcctttcggtaagtgcagtggaagc |
| | tgtacactgcccaggcaaagcgtccgggcagcgtaggcgggcgactcagatc |
| | ccagccagtggacttagcccctgtttgctcctccgataactggggtgacctt |
| | ggttaatattcaccagcagcctcccccgttgcccctctggatccactgctta |
| | aatacggacgaggacagggccctgtctcctcagcttcaggcaccaccactga |
| | cctgggacagt |
| LMTP18 | aggctcagaggcacacaggagtttctgggctcaccctgcccccttccaaccc |
| SEQ ID NO: 131 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggccactacgggtttaggctgcccatgtaaggaggcaaggcctgg |
| | ggacacccgagatgcctggttataattaacccagacatgtggctgccccccc |
| | cccccccaacacctgctgcctctaaaaataaccctgtccctggtggatcccc |
| | tgcatgcgaagatcttcgaacaaggctgtgggggactgagggcaggctgtaa |
| | caggcttgggggccagggcttatacgtgcctgggactcccaaagtattactg |
| | ttccatgttcccggcgaagggccagctgtcccccgccagctagactcagcac |
| | ttagtttaggaaccagtgagcaagtcagcccttggggcagcccatacaaggc |
| | catggggctgggcaagctgcacgcctgggtccggggtgggcacggtgcccgg |
| | gcaacgagctgaaagctcatctgctctcaggggcccctccctggggacagcc |
| | cctcctggctagtcacaccctgtaggctcctctatataacccaggggcacag |
| | gggctgccctcattctaccaccacctccacagcacagacagacactcaggag |
| | ccagccagcgtcgagatcttgctaccagtggaacagccactaaggattctgc |
| | agtgagagcagagggccagctaagtggtactctcccagagactgtctgactc |
| | acgccaccccctccaccttggacacaggacgctgtggtttctgagccaggta |
| | cagtgactcctttcggtaagtgcagtggaagctgtacactgcccaggcaaag |
| | cgtccgggcagcgtaggcgggcgactcagatcccagccagtggacttagccc |
| | ctgtttgctcctccgataactggggtgaccttggttaatattcaccagcagc |
| | ctcccccgttgcccctctggatccactgcttaaatacggacgaggacagggc |
| | cctgtctcctcagcttcaggcaccaccactgacctgggacagt |
| LMTP19 | aggctcagaggcacacaggagtttctgggctcaccctgcccccttccaaccc |
| SEQ ID NO: 132 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggccctgcatgcgaagatcttcgaacaaggctgtgggggactgag |
| | ggcaggctgtaacaggcttgggggccagggcttatacgtgcctgggactccc |
| | aaagtattactgttccatgttcccggcgaagggccagctgtcccccgccagc |
| | tagactcagcacttagtttaggaaccagtgagcaagtcagcccttggggcag |
| | cccatacaaggccatggggctgggcaagctgcacgcctgggtccggggtggg |
| | cacggtgcccgggcaacgagctgaaagctcatctgctctcaggggcccctcc |
| | ctggggacagcccctcctggctagtcacaccctgtaggctcctctatataac |
| | ccaggggcacaggggctgccctcattctaccaccacctccacagcacagaca |
| | gacactcaggagccagccagcgtcgagatcttgctaccagtggaacagccac |
| | taaggattctgcagtgagagcagagggccagctaagtggtactctcccagag |
| | actgtctgactcacgccaccccctccaccttggacacaggacgctgtggttt |
| | ctgagccaggtacagtgactcctttcggtaagtgcagtggaagctgtacact |
| | gcccaggcaaagcgtccgggcagcgtaggcgggcgactcagatcccagccag |
| | tggacttagcccctgtttgctcctccgataactggggtgaccttggttaata |
| | ttcaccagcagcctcccccgttgcccctctggatccactgcttaaatacgga |
| | cgaggacagggccctgtctcctcagcttcaggcaccaccactgacctgggac |
| | agt |
| LMTP20 | aggctcagaggcacacaggagtttctgggctcaccctgcccccttccaaccc |
| SEQ ID NO: 133 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggcccttcagattaaaaataactgaggtaagggcctgggtagggg |
| | aggtggtgtgagacgctcctgtctctcctctatctgcccatcggccctttgg |
| | ggaggaggaatgtgcccaaggactaaaaaaaggccatggagccagaggggcg |
| | agggcaacagacctttcatgggcaaaccttggggccctgctgaagctttggc |
| | ccactacgggtttaggctgcccatgtaaggaggcaaggcctggggacacccg |
| | agatgcctggttataattaacccagacatgtggctgccccccccccccccaa |
| | cacctgctgcctctaaaaataaccctgtccctggtggatcccctgcatgcga |
| | agatcttcgaacaaggctgtgggggactgagggcaggctgtaacaggcttgg |
| | gggccagggcttatacgtgcctgggactcccaaagtattactgttccatgtt |
| | cccggcgaagggccagctgtcccccgccagctagactcagcacttagtttag |
| | gaaccagtgagcaagtcagcccttggggcagcccatacaaggccatggggct |
| | gggcaagctgcacgcctgggtccggggtgggcacggtgcccgggcaacgagc |
| | tgaaagctcatctgctctcaggggcccctccctggggacagcccctcctggc |
| | tagtcacaccctgtaggctcctctatataacccaggggcacaggggctgccc |
| | tcattctaccaccacctccacagcacagacagacactcaggagccagccagc |
| | gtcgagatcttgctaccagtggaacagccactaaggattctgcagtgagagc |
| | agagggccagctaagtggtactctcccagagactgtctgactcacgccaccc |
| | cctccaccttggacacaggacgctgtggtttctgagccaggtacagtgactc |
| | ctttcggtaagtgcagtggaagctgtacactgcccaggcaaagcgtccgggc |
| | agcgtaggcgggcgactcagatcccagccagtggacttagcccctgtttgct |
| | cctccgataactggggtgaccttggttaatattcaccagcagcctcccccgt |
| | tgcccctctggatccactgcttaaatacggacgaggacagggccctgtctcc |
| | tcagcttcaggcaccaccactgacctgggacagt |
| ApoE.hAAT | aggctcagaggcacacaggagtttctgggctcaccctgcccccttccaaccc |
| SEQ ID NO: 134 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagaggggtacccggggatcttgctaccagtggaacagccactaaggatt |
| | ctgcagtgagagcagagggccagctaagtggtactctcccagagactgtctg |
| | actcacgccaccccctccaccttggacacaggacgctgtggtttctgagcca |
| | ggtacaatgactcctttcggtaagtgcagtggaagctgtacactgcccaggc |
| | aaagcgtccgggcagcgtaggcgggcgactcagatcccagccagtggactta |
| | gcccctgtttgctcctccgataactggggtgaccttggttaatattcaccag |
| | cagcctcccccgttgcccctctggatccactgcttaaatacggacgaggaca |
| | gggccctgtctcctcagcttcaggcaccaccactgacctgggacagt |
| Alpha-Mic/Bik | aggttaatttttaaaaagcagtcaaaagtccaagtggcccttggcagcattt |
| Enhancer | actctctctgtttgctctggttaataatctcaggagcacaaacattcc |
| (Mic/BikE) | |
| SEQ ID NO: 135 | |
| Tandem (2) | aggttaatttttaaaaagcagtcaaaagtccaagtggcccttggcagcattt |
| alpha-Mic/Bik | actctctctgtttgctctggttaataatctcaggagcacaaacattccaggt |
| Enhancers | taatttttaaaaagcagtcaaaagtccaagtggcccttggcagcatttactc |
| (2 Mic/BikE) | tctctgtttgctctggttaataatctcaggagcacaaacattcc |
| SEQ ID NO: 136 | |
| ApoE Hepatic | aggctcagaggcacacaggagtttctgggctcaccctgcccccttccaaccc |
| Control Region | ctcagttcccatcctccagcagctgtttgtgtgctgcctctgaagtccacac |
| containing ApoE | tgaacaaacttcagcctactcatgtccctaaaatgggcaaacattgcaagca |
| Enhancer | gcaaacagcaaacacacagccctccctgcctgctgaccttggagctggggca |
| SEQ ID NO: 137 | gaggtcagagacctctctgggcccatgccacctccaacatccactcgacccc |
| | ttggaatttcggtggagaggagcagaggttgtcctggcgtggtttaggtagt |
| | gtgagaggg |
| Tandem (2) | aggctcagaggcacacaggagtttctgggctcaccctgcccccttccaaccc |
| ApoE Enhancers | ctcagttcccatcctccagcagctgtttgtgtgctgcctctgaagtccacac |
| SEQ ID NO: 138 | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggtctagaaggctcagaggcacacaggagtttctgggctcaccct |
| | gcccccttccaacccctcagttcccatcctccagcagctgtttgtgtgctgc |
| | ctctgaagtccacactgaacaaacttcagcctactcatgtccctaaaatggg |
| | caaacattgcaagcagcaaacagcaaacacacagccctccctgcctgctgac |
| | cttggagctggggcagaggtcagagacctctctg |
| hAAT Promoter | gatcttgctaccagtggaacagccactaaggattctgcagtgagagcagagg |
| SEQ ID NO: 139 | gccagctaagtggtactctcccagagactgtctgactcacgccaccccctcc |
| | accttggacacaggacgctgtggtttctgagccaggtaca [symbol] actcctttc |
| | ggtaagtgcagtggaagctgtacactgcccaggcaaagcgtccgggcagcgt |
| | aggcgggcgactcagatcccagccagtggacttagcccctgtttgctcctcc |
| | gataactggggtgaccttggttaatattcaccagcagcctcccccgttgccc |
| | ctctggatccactgcttaaatacggacgaggacagggccctgtctcctcagc |
| | ttcaggcaccaccactgacctgggacagt |
| hAAT(AATG) | gatcttgctaccagtggaacagccactaaggattctgcagtgagagcagagg |
| Promoter | gccagctaagtggtactctcccagagactgtctgactcacgccaccccctcc |
| SEQ ID NO: 140 | accttggacacaggacgctgtggtttctgagccaggtaca [symbol] actcctttc |
| | ggtaagtgcagtggaagctgtacactgcccaggcaaagcgtccgggcagcgt |
| | aggcgggcgactcagatcccagccagtggacttagcccctgtttgctcctcc |
| | gataactggggtgaccttggttaatattcaccagcagcctcccccgttgccc |
| | ctctggatccactgcttaaatacggacgaggacagggccctgtctcctcagc |
| | ttcaggcaccaccactgacctgggacagt |
| Mck Enhancer | ccactacgggtttaggctgcccatgtaaggaggcaaggcctggggacacccg |
| (MckE) | agatgcctggttataattaacccagacatgtggctgccccccccccccccaa |
| SEQ ID NO: 141 | cacctgctgcctctaaaaataaccctgtccctggtggatc |
| Tandem (2) Mck | ccactacgggtttaggctgcccatgtaaggaggcaaggcctggggacacccg |
| Enhancers | agatgcctggttataattaacccagacatgtggctgccccccccccccccaa |
| (2 MckE) | cacctgctgcctctaaaaataaccctgtccctggtggatcccactacgggtt |
| SEQ ID NO: 142 | taggctgcccatgtaaggaggcaaggcctggggacacccgagatgcctggtt |
| | ataattaacccagacatgtggctgccccccccccccccaacacctgctgcct |
| | ctaaaaataaccctgtccctggtggatc |
| Tandem Mck (3) | ccactacgggtttaggctgcccatgtaaggaggcaaggcctggggacacccg |
| Enhancers | agatgcctggttataattaacccagacatgtggctgccccccccccccccaa |
| (3 MckE) | cacctgctgcctctaaaaataaccctgtccctggtggatcccactacgggtt |
| SEQ ID NO: 143 | taggctgcccatgtaaggaggcaaggcctggggacacccgagatgcctggtt |
| | ataattaacccagacatgtggctgccccccccccccccaacacctgctgcct |
| | ctaaaaataaccctgtccctggtggatcccactacgggtttaggctgcccat |
| | gtaaggaggcaaggcctggggacacccgagatgcctggttataattaaccca |
| | gacatgtggctgccccccccccccccaacacctgctgcctctaaaaataacc |
| | ctgtccctggtggatc |
| Myosin heavy | cccttcagattaaaaataactgaggtaagggcctgggtaggggaggtggtgt |
| chain enhancer | gagacgctcctgtctctcctctatctgcccatcggccctttggggaggagga |
| (MhcE) | atgtgcccaaggactaaaaaaaggccatggagccagaggggcgagggcaaca |
| SEQ ID NO: 144 | gacctttcatgggcaaaccttggggccctgctgaagctttggc |

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron (e.g. VH4 intron (SEQ ID NO:57) or a chimeric intron (SEQ ID NO:56). The viral vectors may also include a Kozak sequence to promote translation of the transgene product, for example GCCACC.

In certain embodiments, the viral vectors provided herein comprise a polyadenylation sequence downstream of the coding region of the transgene. Any polyA site that signals termination of transcription and directs the synthesis of a polyA tail is suitable for use in AAV vectors of the present disclosure. Exemplary polyA signals are derived from, but not limited to, the following: the SV40 late gene, the rabbit β-globin gene (SEQ ID NO:55), the bovine growth hormone (BPH) gene, the human growth hormone (hGH) gene, the synthetic polyA (SPA) site, and the bovine growth hormone (bGH) gene. See, e.g., Powell and Rivera-Soto, 2015, Discov. Med., 19(102):49-57.

Provided are gene expression cassettes and rAAVs comprising gene expression cassettes in which expression of the transgene is controlled by engineered nucleic acid regulatory elements that have more than one regulatory element (promoter or enhancer).

### 5.2.4 Signal Peptides

In certain embodiments, the vectors provided herein comprise components that modulate protein delivery. In certain embodiments, the viral vectors provided herein comprise one or more signal peptide encoding nucleotide sequences that are fused to the nucleotide sequence encoding the anti-TNFα Fc fusion protein such that the protein expressed from the transgene has the signal sequence at the N-terminus to direct secretion. Signal peptides may also be referred to herein as “leader sequences” or “leader peptides”. In certain embodiments, the signal peptides allow for the transgene product to achieve the proper packaging (e.g., glycosylation) in the cell. In certain embodiments, the signal peptides allow for the transgene product to achieve the proper localization in the cell. In certain embodiments, the signal peptides allow for the transgene product to achieve secretion from the cell.

There are two general approaches to select a signal sequence for protein production in a gene therapy context or in cell culture. One approach is to use a signal peptide from proteins homologous to the protein being expressed. For example, a human antibody signal peptide may be used to express IgGs in CHO or other cells. Another approach is to identify signal peptides optimized for the particular host cells used for expression. Signal peptides may be interchanged between different proteins or even between proteins of different organisms, but usually the signal sequences of the most abundant secreted proteins of that cell type are used for protein expression. For example, the signal peptide of human albumin, the most abundant protein in plasma, was found to substantially increase protein production yield in CHO cells. However, certain signal peptides may retain function and exert activity after being cleaved from the expressed protein as “post-targeting functions”. Thus, in specific embodiments, the signal peptide is selected from signal peptides of the most abundant proteins secreted by the cells used for expression to avoid the post-targeting functions. In a certain embodiment, the signal sequence is fused to both the heavy and light chain sequences. An exemplary sequence is MYRMQLLLLIALSLALVTNS (SEQ ID NO:62) which can be encoded by a nucleotide sequence of SEQ ID NO:63 (see Table 2, FIGS. 2A and B). Alternatively, signal sequences that are appropriate for expression, and may cause selective expression or directed expression of the HuPTM anti-TNFα Fc fusion protein in eye (ocular tissues) are provided in Table 2 below. Also provided are signal sequences active in liver (Table 3) and muscle (Table 4) cells.

TABLE 2
Signal peptides for expression in eye

| | SEQ ID | |
|---|---|---|
| Signal Peptide Origin | NO: | Sequence |
| Mutant interleukin 2 | 62 | MYRMQLLLLIALSLALVINS |
| signal peptide | | |
| Mutant interleukin 2 | 63 | atgtataggatgcaactgctcctcctgattgctctgagcctggctcttgtg |
| signal peptide coding | | accaactct |
| sequence | | |
| VEGF-A signal | 64 | MNFLLSWVHWSLALLLYLHHAKWSQA |
| peptide | | |
| Fibulin-1 signal | 65 | MERAAPSRRVPLPLLLLGGLALLAAGVDA |
| peptide | | |
| Vitronectin signal | 66 | MAPLRPLLILALLAWVALA |
| peptide | | |
| Complement Factor H | 67 | MRLLAKIICLMLWAICVA |
| signal peptide | | |
| Opticin signal peptide | 68 | MRLLAFLSLLALVLQETGT |
| Albumin signal | 69 | MKWVTFISLLFLFSSAYS |
| peptide | | |
| Chymotrypsinogen | 70 | MAFLWLLSCWALLGTTFG |
| signal peptide | | |
| Interleukin-2 signal | 71 | MYRMQLLSCIALILALVINS |
| peptide | | |
| Trypsinogen-2 signal | 72 | MNLLLILTFVAAAVA |
| peptide | | |

TABLE 3
Signal peptides for expression in liver cells.

| | SEQ ID | |
|---|---|---|
| Signal Peptide Origin | NO: | Sequence |
| Human Serum albumin | 73 | MKWVTFISLLFLFSSAYS |
| Human α-1 Antitrypsin | 74 | MPSSVSWGILLLAGLCCLVPVSLA |
| (SERPINA1) | | |
| Human Apolipoprotein | 75 | MKAAVLTLAVLFLTGSQA |
| A-1 | | |
| Human Apolipoprotein | 76 | MKLLAATVLLLTICSLEG |
| A-2 | | |
| Human Apolipoprotein | 77 | MDPPRPALLALLALPALLLLLLAGARA |
| B-100 | | |
| Human Coagulation | 78 | MQRVNMIMAESPGLITICLLGYLLSAEC |
| Factor IX | | |
| Human Complement | 79 | MGPLMVLFCLLFLYPGLADS |
| C2 | | |
| Human Complement | 80 | MWLLVSVILISRISSVGG |
| Factor H-related | | |
| Protein 2 (CFHR2) | | |
| Human Complement | 81 | MLLLFSVILISWVSTVGG |
| Factor H-related | | |
| Protein 5 (CFHR5) | | |
| Human Fibrinogen α- | 82 | MFSMRIVCLVLSVVGTAWT |
| chain (FGA) | | |
| Human Fibrinogen ß- | 83 | MKRMVSWSFHKLKTMKHLLLLLLCVFLVKS |
| chain (FGB) | | |
| Human Fibrinogen γ- | 84 | MSWSLHPRNLILYFYALLFLSSTCVA |
| chain (FGG) | | |
| Human α-2-HS- | 85 | MKSLVLLLCLAQLWGCHS |
| Glycoprotein (AHSG) | | |
| Human Hemopexin | 86 | MARVLGAPVALGLWSLCWSLAIA |
| (HPX) | | |
| Human Kininogen-1 | 87 | MKLITILFLCSRLLLSLT |
| Human Mannose- | 88 | MSLFPSLPLLLLSMVAASYS |
| binding protein C | | |
| (MBL2) | | |
| Human Plasminogen | 89 | MEHKEVVLLLLLFLKSGQG |
| (PLMN) | | |
| Human Prothrombin | 90 | MAHVRGLQLPGCLALAALCSLVHS |
| (Coagulation Factor II) | | |
| Human Secreted | 91 | MISRMEKMTMMMKILIMFALGMNYWSCSG |
| Phosphoprotein 24 | | |
| Human Anti-thrombin- | 92 | MYSNVIGTVTSGKRKVYLLSLLLIGFWDCVTC |
| III (SERPINC1) | | |
| Human Serotransferrin | 93 | MRLAVGALLVCAVLGLCLA |
| (TF) | | |

TABLE 4
Signal peptides for expression in muscle cells.

| | SEQ ID | |
|---|---|---|
| Signal Peptide Origin | NO: | Sequence |
| Human SPARC | 94 | MRAWIFFLLCLAGRALA |
| Human Collagen alpha-1 (I) | 95 | MFSFVDLRLLLLLAATALLTHG |
| chain | | |
| Human Lactotransferrin | 96 | MKLVFLVLLFLGALGLCLA |
| Human Complement C3 | 97 | MGPTSGPSLLLLLLTHLPLALG |
| Human Lumican | 98 | MSLSAFTLFLALIGGTSG |
| Human Gelsolin isoform 1 | 99 | MAPHRPAPALLCALSLALCALSLPVRA |
| Human Pro-cathepsin H | 100 | MWATLPLLCAGAWLLGVPVCGA |
| Human SERPINF1 | 101 | MQALVLLLCIGALLGHSSC |
| Human SERPINE1 | 102 | MQMSPALTCLVLGLALVFGEGSA |
| Human Cathepsin D | 103 | MQPSSLLPLALCLLAAPASA |
| Human TIMP 1 | 104 | MAPFEPLASGILLLLWLIAPSRA |
| Human Fibronectin | 105 | MLRGPGPGLLLLAVQCLGTAVPSTGASKSKR |
| Human Complement Cls | 106 | MWCIVLFSLLAWVYA |
| subcomponent | | |
| Human Cathepsin L1 | 107 | MNPTLILAAFCLGIASA |
| Human Cathepsin B | 108 | MWQLWASLCCLLVLANA |
| Human Salivary acidic | 109 | MLLILLSVALLAFSSA |
| proline-rich | | |
| phosphoprotein 1/2 | | |
| Human Follistatin-related | 110 | MWKRWLALALALVAVAWVRA |
| protein 1 | | |

### 5.2.5 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene. In certain embodiments, the UTRs are optimized for the stability of the mRNA of the transgene. In certain embodiments, the UTRs are optimized for the secondary structure of the mRNA of the transgene.

### 5.2.6 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety). In preferred embodiments, nucleotide sequences encoding the ITRs may, for example, comprise the nucleotide sequences of SEQ ID NOS:58 (5′-ITR) or 60 (3′-ITR). In certain embodiments, the modified ITRs used to produce self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety). In preferred embodiments, nucleotide sequences encoding the modified ITRs may, for example, comprise the nucleotide sequences of SEQ ID NOS:58 (5′-ITR) or 60 (3′-ITR) or modified for scAAV, SEQ ID NO:59 (m 5′ITR) or SEQ ID NO:61 (m 3′ ITR).

### 5.2.7 Transgenes

The transgenes encode a HuPTM anti-TNFα Fc fusion protein, e.g. a TNFR1:Fc fusion protein or TNFR2:Fc fusion protein, based upon a therapeutic anti-TNFα Fc fusion protein disclosed herein. In specific embodiments, the HuPTM anti-TNFα Fc fusion protein is engineered to contain additional glycosylation sites on the TNFR and/or Fc domain. In addition, the Fc domain of the anti-TNFα Fc fusion protein may be engineered to alter the glycosylation site at N297 to prevent glycosylation at that site (for example, a substitution at N297 for another amino acid and/or a substitution at T297 for a residue that is not a T or S to knock out the glycosylation site). Such Fc domains are “aglycosylated”.

### 5.2.7.1 Constructs for Expression of HuPTM TNFR:Fc Fusion Proteins

In certain embodiments, the transgene encodes an anti-TNFa fusion protein, wherein the anti-TNFα Fc fusion protein comprises a soluble, extracellular portion of human TNFα receptor type I (TNFR1) or type II (TNFR2) covalently linked, such as through a peptide bond, to polypeptide containing an Fc domain of an immunoglobulin heavy chain.

In certain embodiments, the transgene encodes all or a TNFα-binding portion of the extracellular domain of the TNFR2 (SEQ ID NO: 2) fused to all or a portion of an Fc domain (including, for example, the hinge, CH2 and CH3 domains) of an immunoglobulin heavy chain (SEQ ID NO: 3 or 4). In preferred embodiments, the transgene encodes an anti-TNFα Fc fusion protein, wherein the anti-TNFα Fc fusion protein comprises human soluble, extracellular domain of TNFR2 covalently linked, such as through a peptide bond, to the Fc domain of human IgG1 (including heavy chain CH2, and heavy chain CH3, and the hinge region) that upon expression associate to form a dimeric TNFR2:Fc fusion protein. In particular, the anti-TNFα Fc fusion protein is etanercept (having the amino acid sequence of SEQ ID NO: 10 or 11 with leader sequence) or any biosimilar form of etanercept, including etanercept-szzs (ERELZI®) and etantercept-ykro (ETICOVO®).

In other embodiments, the transgene encodes all or part of the soluble extracellular domain of the TNFR1 (SEQ ID NO: 1) fused to all or part of an immunoglobulin heavy chain Fc domain (SEQ ID NO: 6 or 7). In preferred embodiments, the transgene encodes an anti-TNFα Fc fusion protein, wherein the anti-TNFα Fc fusion protein comprises human soluble, extracellular domain of TNFR1 covalently linked, such as through a peptide bond, to the Fc portion of human IgGI (including heavy chain CH2, and heavy chain CH3, and the hinge region), that upon expression associate to form a dimeric TNFR1:Fc fusion protein (SEQ ID NO: 12). In some embodiments, TNFR1 is fused to the Fc domain via a thrombin cleavage site LVPRGS (SEQ ID NO:8). In certain embodiments the anti-TNFα Fc fusion protein is EYS060 (having an amino acid sequence of SEQ ID NO: 12).

The recombinant AAV constructs express the HuPTM anti-TNFα Fc fusion protein in a cell, cell culture, or in a subject. The nucleotide sequences encoding the soluble extracellular domain of the TNFR and Fc domains may be codon optimized for expression in human cells and have reduced incidence of CpG dimers in the sequence to promote expression in human cells. In preferred embodiments, the transgenes encode any of the therapeutic anti-TNFα Fc fusion proteins disclosed herein, for example, the TNFR:Fc fusion depicted in FIG. 1 or 2 (sequences provided in FIGS. 2A and 2B) herein.

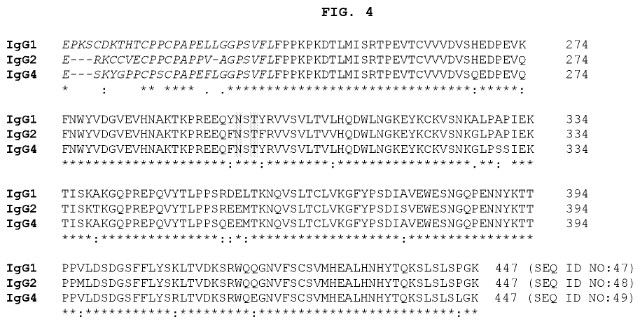

In certain embodiments, the Fc domain is an IgG Fc domain, but in other embodiments, the Fc region may be an IgA, IgD, IgE, or IgM. The Fc fusion protein expressed from the transgene may have an IgG1, IgG2, IgG3, or IgG4 Fc domain, but is preferably derived from human IgG1. The Fc domain can include some or all of the heavy chain CH1, hinge, CH2, and CH3 domains described herein (see FIG. 4 and Table 5) or fragments or variants thereof.

The Fc domain of the Fc fusion protein has one or more effector functions that vary with the antibody isotype. The effector functions can be the same as that of the wild-type or the therapeutic Fc fusion protein or can be modified therefrom to add, enhance, modify, or inhibit one or more effector functions using the Fc modifications disclosed in Section 5.2.8, infra. In certain embodiments, the HuPTM non-monoclonal antibody transgene encodes a fusion protein comprising an Fc polypeptide comprising an amino acid sequence that is at least 85%, 86%, 87%, 88%, 89%, 90%, 91%, 92%, 93%, 94%, 95%, 96%, 97%, 98% or 99% identical to the sequence set forth in the Fc domain polypeptides of the therapeutic fusion protein described herein as set forth in Table 6 for etanercept or an exemplary Fc domain of an IgG1, IgG2 or IgG4 isotype as set forth in Table 5. In some embodiments, the HuPTM fusion protein comprises an Fc polypeptide or a sequence that is a variant of the Fc polypeptide sequence in Table 5 in that the sequence has been modified with one or more of the techniques described in Section 5.2.8 infra, to alter the Fc polypeptide's effector function.

The recombinant AAV constructs express the HuPTM anti-TNFα Fc fusion protein in a cell, cell culture, or in a subject. The nucleotide sequences encoding the anti-TNFα Fc fusion protein may be codon optimized for expression in human cells and have reduced incidence of CpG dimers in the sequence to promote expression in human cells. In preferred embodiments, the transgene encodes any of the therapeutic anti-TNFα Fc fusion protein disclosed herein, for example, the therapeutic anti-TNFα Fc fusion protein etanercept or EYS606 depicted in FIGS. 2A and 2B herein and including, in certain embodiments, the associated Fc domain provided in Table 6 or, alternatively in Table 5. The Fc domain is further discussed in Section 5.2.8. Exemplary Fc domain sequences are provided in Table 6, as well as in FIG. 3 and Table 5. FIG. 2A provides the amino acid sequence of TNFR2:Fc fusion etanercept and FIG. 2B provides the amino acid sequence of TNFR1:Fc fusion EYS606 (see also Table 6).

In some embodiments, the transgene for expression of an anti-TNFα Fc fusion protein may comprises a nucleotide sequence encoding a soluble, extracellular portion of human TNFα receptor type I (TNFR1) or type II (TNFR2) covalently linked, such as through a peptide bond, to polypeptide containing an Fc domain of an immunoglobulin heavy chain as described further herein. In some embodiments, TNFR1 is fused to the Fc domain via a thrombin cleavage site (SEQ ID NO:8).

Nucleotide sequences encoding the soluble extracellular domain of TNFR1 or TNFR2, optionally the thrombin cleavage site, the hinge region, and the IgG1 Fc chain (CH2+CH3) of a therapeutic anti-TNFα Fc fusion protein as disclosed herein are provided in Table 7. In certain embodiments, these nucleotide sequences are codon optimized for expression in human cells. See for example, the codon optimized sequences of etanercept (SEQ ID NOs 15-18) of Table 7.

The transgene may include the portion of the hinge region that forms interchain di-sulfide bonds (e.g., the portion containing the sequence CPPCPA (SEQ ID NO:153). Fc domain sequences that do not contain portion of the hinge region comprising the sequence CCPCPA (SEQ ID NO:153) will not form intrachain disulfide bonds, whereas those Fc fusion domain sequences with a portion of the hinge region at the C-terminus containing the sequence CPPCPA (SEQ ID NO:153) will form intrachain disulfide bonds and, thus, will form dimeric fusion proteins fragments. In some embodiments, the hinge contains all or a portion of the amino acid sequence EPKSCDKTHTCPPCPAPELLGG (SEQ ID NO:145), and specifically, EPKSCDKTHL (SEQ ID NO:146), EPKSCDKTHT (SEQ ID NO:147), EPKSCDKTHTCPPCPA (SEQ ID NO:148), EPKSCDKTHLCPPCPA (SEQ ID NO:149), DKTHTCPPCPAPELLGG (SEQ ID NO:21), DKTHTCPPCPAPELLGGPSVFL (SEQ ID NO:152), EPKSCDKTHTCPPCPAPELLGGPSVFL (SEQ ID NO:150) or EPKSCDKTHLCPPCPAPELLGGPSVFL (SEQ ID NO:151).

In certain embodiments, the viral vectors provided herein comprise the following elements in the following order: a) a constitutive or inducible (e.g., hypoxia-inducible or rifamycin-inducible) promoter sequence or a tissue specific promoter/regulatory region, for example, one of the regulatory regions provided in Table 1 or Tablela, particularly a promoter that promotes ocular tissue specific expression and b) a sequence encoding the transgene (e.g., an anti-TNFα Fc fusion protein). In certain embodiments, the sequence comprising the transgene encodes an anti-TNFα Fc fusion protein comprising a soluble, extracellular portion of human TNFα receptor type I (TNFR1) or type II (TNFR2) covalently linked, such as through a peptide bond, to polypeptide containing an Fc domain of an immunoglobulin heavy chain. In certain embodiments, the sequence comprising the transgene encodes a signal sequence at the N-terminus of the anti-TNFα Fc fusion protein that directs secretion and post translational modification in said human ocular cells. In other embodiments, the sequence encodes for a transgene product having an amino acid sequence of SEQ ID NO: 10, 11 or 12. In preferred embodiments, the anti-TNFα fusion protein is etanercept or EYS606.

In certain embodiments, the viral vectors provided herein comprise the following elements in the following order: a) a constitutive or an inducible promoter sequence or a tissue specific promoter, such as one of the promoters or regulatory regions in Table 1 and 1a, and b) a sequence encoding the transgene (e.g., an anti-TNFα Fc fusion protein), wherein the transgene comprises a signal peptide, a soluble, extracellular portion of human TNFα receptor type I (TNFR1) or type II (TNFR2) covalently linked, such as through a peptide bond, to polypeptide comprising an Fc domain of an immunoglobulin heavy chain to form an anti-TNFα Fc fusion protein. In other embodiments, the viral vectors provided herein have the following elements in the following order: a) a ocular tissue-specific promoter listed in Table 1 and 1a, and b) a sequence encoding the transgene comprising a signal peptide, wherein the transgene comprises a signal peptide, a soluble, extracellular portion of human TNFα receptor type I (TNFR1) or type II (TNFR2) covalently linked, such as through a peptide bond, to polypeptide containing an Fc domain of an immunoglobulin heavy chain to form anti-TNFα Fc fusion protein. In specific embodiments, the viral vectors provided herein comprise further a thrombin cleavage site linked to the C-terminus of the TNFR domain having amino acid sequence of LSPRGS (SEQ ID NO:158).

In specific embodiments, provided are AAV vectors comprising a viral capsid that is at least 95% identical to the amino acid sequence of an AAV2.7m8 capsid, AAV8 capsid, an AAV3B capsid, or an AAVrh73 capsid (see FIG. 3); and an artificial genome comprising an expression cassette flanked by AAV inverted terminal repeats (ITRs), wherein the expression cassette comprises a transgene encoding an anti-TNFα Fc fusion protein; operably linked to one or more regulatory sequences that control expression of the transgene in one or more ocular tissues.

The rAAV vectors that encode and express the therapeutic fusion protein may be administered to treat or prevent or ameliorate symptoms of a disease or condition amenable to treatment, prevention or amelioration of symptoms with the therapeutic fusion protein. Also provided are methods of expressing HuPTM anti-TNFα Fc fusion protein in human cells using the rAAV vectors and constructs encoding them.

### 5.2.8 Fc Region Modifications

In certain embodiments, the transgenes encode anti-TNFα Fc fusion proteins that associate to form a dimeric Fc fusion protein. Accordingly, the transgenes comprise nucleotide sequences that encode, for example, the soluble, extracellular portion of human TNFα receptor type I (TNFR1) or type II (TNFR2) covalently linked, such as through a peptide bond, to polypeptide containing an Fc domain of an immunoglobulin heavy chain (FIGS. 2A and 2B), including all or a portion of the hinge region of the heavy chain and N-terminal of the Fc domain peptide. Table 6 provides the amino acid sequences of the Fc polypeptides for certain of the therapeutic anti-TNFα Fc fusion proteins described herein. Alternatively, an IgG2, or IgG4 Fc domain, the sequences of which are provided in FIG. 4 and provided in Table 5 may be utilized. As detailed, the transgene may comprise a nucleotide sequence encoding the Fc domain polypeptide for the therapeutic anti-TNFα Fc fusion protein linked to the nucleotide sequence encoding the soluble, extracellular portion of human TNFR at the N-terminus of the hinge region as provided herein (with the amino acid sequences provided in FIGS. 2A and 2B and Table 6).

The term “Fc region” refers to a dimer of two “Fc polypeptides” (or “Fc domains”), each “Fc polypeptide” comprising the heavy chain constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to at least the last two constant region immunoglobulin domains of IgA, IgD, and IgG, or the last three constant region immunoglobulin domains of IgE and IgM and may also include part or all of the flexible hinge N-terminal to these domains. For IgG, e.g., “Fc polypeptide” comprises immunoglobulin domains Cgamma2 (Cγ2, often referred to as CH2 domain) and Cgamma3 (Cγ3, also referred to as CH3 domain) and may include the lower part of the hinge domain between Cgammal (Cγ1, also referred to as CH1 domain) and CH2 domain. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, e.g., Fc polypeptide comprises immunoglobulin domains Calpha2 (Cα2) and Calpha3 (Cα3) and may include the lower part of the hinge between Calphal (Cα1) and Cα2.

In certain embodiments, the Fc polypeptide is that of etanercept or EYS606 (see Table 6). In other embodiments, the Fc polypeptide is an IgG Fc polypeptide. The Fc polypeptide may be from the IgG1, IgG2, or IgG4 isotype (see FIG. 4 for alignment of IgG1, IgG2 and IgG4 Fc domain sequences, numbered according to EU numbering) or may be an IgG3 Fc domain, depending, for example, upon the desired effector activity of the therapeutic antibody. In some embodiments, the engineered heavy chain constant region (CH), which includes the Fc domain, is chimeric. As such, a chimeric CH region combines CH domains derived from more than one immunoglobulin isotype and/or subtype. For example, the chimeric (or hybrid) CH region comprises part or all of an Fc region from IgG, IgA and/or IgM. In other examples, the chimeric CH region comprises part or all a CH2 domain derived from a human IgG1, human IgG2, or human IgG4 molecule, combined with part or all of a CH3 domain derived from a human IgG1, human IgG2, or human IgG4 molecule. In other embodiments, the chimeric CH region contains a chimeric hinge region.

TABLE 5
Table of Fc Domain Amino Acid Sequences

| | Chain/ | |
|---|---|---|
| | SEQ ID | |
| Fc domains | NO. | Sequence |
| IgG1 | 47 | EPKSCDKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| IgG2 | 48 | ERKCCVECPPCPAPPVAGPSVFLFPPKPKDTLMISRTPEVTCVVVDVSHEDPEVqFNW |
| | | YVDGVEVHNAKTKPREEQFNSTFRVVSVLTVVHQDWLNGKEYKCKVSNKGLPAPIEKT |
| | | ISKTKGQPREPQVYTLPPSREEMTKNQVSLTCLVKGFYPSDISVEWESNGQPENNYKT |
| | | TPPMLDSDGSFFLYSKLTVDKSRWQQGNVFSCSVMHEALHNHYTQKSLSLSPGK |
| IgG4 | 49 | ESKYGPPCPSCPAPEFLGGPSVFLFPPKPKDTLMISRTPEVTCVVVDVSQEDPEVqEN |
| | | WYVDGVEVHNAKTKPREEQFNSTYRVVSVLTVLHQDWLNGKEYKCKVSNKGLPSSIEK |
| | | TISKAKGQPREPQVYTLPPSQEEMTKNQVSLTCLVKGFYPSDIAVEWESNGQPENNYK |
| | | TTPPVLDSDGSFFLYSRLTVDKSRWQEGNVFSCSVMHEALHNHYTQKSLSLSLGK |

In some embodiments, the recombinant vectors encode therapeutic Fc fusions comprising an engineered (mutant) Fc regions, e.g. engineered Fc regions of an IgG constant region. Modifications to an antibody constant region, Fc region or Fc fragment of an IgG antibody may alter one or more effector functions such as Fc receptor binding or neonatal Fc receptor (FcRn) binding and thus half-life, CDC activity, ADCC activity, and/or ADPC activity, compared to a corresponding antibody having a wild-type IgG constant region, or an IgG heavy chain constant region without the recited modification(s). Accordingly, in some embodiments, the antibody may be engineered to provide an antibody constant region, Fc region or Fc fragment of an IgG antibody that exhibits altered binding (as compared to a reference or wild-type constant region without the recited modification(s)) to one or more Fc receptors (e.g., FcγRI, FcγRIIA, FcγRIIB, FcγRIIIA, FcγRIIIB, FcγRIV, or FcRn receptor). In some embodiments, the antibody an antibody constant region, Fc region or Fc fragment of an IgG antibody that exhibits a one or more altered effector functions such as CDC, ADCC, or ADCP activity, compared to a corresponding antibody having a wild-type IgG constant region, or an IgG constant without the recited modification(s).

“Effector function” refers to a biochemical event that results from the interaction of an antibody Fc region with an Fc receptor or ligand. Effector functions include FcγR-mediated effector functions such as ADCC and ADCP and complement-mediated effector functions such as CDC.

An “effector cell” refers to a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and T cells, and may be from any organism including but not limited to humans, mice, rats, rabbits, and monkeys.

“ADCC” or “antibody dependent cell-mediated cytotoxicity” refers to the cell-mediated reaction wherein nonspecific cytotoxic effector (immune) cells that express FcγRs recognize bound antibody on a target cell and subsequently cause lysis of the target cell.

“ADCP” or “antibody dependent cell-mediated phagocytosis” refers to the cell-mediated reaction wherein nonspecific cytotoxic effector (immune) cells that express FcγRs recognize bound antibody on a target cell and subsequently cause phagocytosis of the target cell.

“CDC” or “complement-dependent cytotoxicity” refers to the reaction wherein one or more complement protein components recognize bound antibody on a target cell and subsequently cause lysis of the target cell.

In some embodiments, the modifications of the Fc domain include, but are not limited to, the following modifications and combinations thereof, with reference to EU numbering of an IgG constant region (see FIG. 4): 233, 234, 235, 236, 237, 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

In certain embodiments, the Fc region comprises an amino acid addition, deletion, or substitution of one or more of amino acid residues 251-256, 285-290, 308-314, 385-389, and 428-436 of the IgG. In some embodiments, 251-256, 285-290, 308-314, 385-389, and 428-436 (EU numbering of Kabat; see FIG. 4) is substituted with histidine, arginine, lysine, aspartic acid, glutamic acid, serine, threonine, asparagine, or glutamine. In some embodiments, a non-histidine residue is substituted with a histidine residue. In some embodiments, a histidine residue is substituted with a non-histidine residue.

Enhancement of FcRn binding by an antibody having an engineered Fc leads to preferential binding of the affinity-enhanced antibody to FcRn as compared to antibody having wild-type Fc, and thus leads to a net enhanced recycling of the FcRn-affinity-enhanced antibody, which results in further increased antibody half-life. An enhanced recycling approach allows highly effective targeting and clearance of antigens, including e.g. “high titer” circulating antigens, such as C5, cytokines, or bacterial or viral antigens.

Provided in certain embodiments are modified constant region, Fc region or Fc fragment of an IgG antibody with enhanced binding to FcRn in serum as compared to a wild-type Fc region (without engineered modifications). In some instances, antibodies, e.g. IgG antibodies, are engineered to bind to FcRn at a neutral pH, e.g., at or above pH 7.4, to enhance pH-dependence of binding to FcRn as compared to a wild-type Fc region (without engineered modifications). In some instances, antibodies, e.g. IgG antibodies, are engineered to exhibit enhanced binding (e.g. increased affinity or K_{D}) to FcRn in endosomes (e.g., at an acidic pH, e.g., at or below pH 6.0) relative to a wild-type IgG and/or reference antibody binding to FcRn at an acidic pH, as well as in comparison to binding to FcRn in serum (e.g., at a neutral pH, e.g., at or above pH 7.4). Provided are antibodies with an engineered antibody constant region, Fc region or Fc fragment of an IgG antibody that exhibits an improved serum or resident tissue half-life, compared to a corresponding antibody having a wild-type IgG constant region, or an IgG constant without the recited modification(s);

Non-limiting examples of such Fc modifications include, e.g., a modification at position 250 (e.g., E or Q); 250 and 428 (e.g., L or F); 252 (e.g., LN/Y/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P) (EU numbering; see FIG. 4).

In some embodiments, the Fc region can be a mutant form such as hIgG1 Fc including M252 mutations, e.g. M252Y and S254T and T256E (“YTE mutation”) exhibit enhanced affinity for human FcRn (Dall'Acqua, et al., 2002, J Immunol 169:5171-5180) and subsequent crystal structure of this mutant antibody bound to hFcRn resulting in the creation of two salt bridges (Oganesyan, et al. 2014, JBC 289(11): 7812-7824). Antibodies having the YTE mutation have been administered to monkeys and humans, and have significantly improved pharmacokinetic properties (Haraya, et al., 2019, Drug Metabolism and Pharmacokinetics, 34(1):25-41).

In some embodiments, modifications to one or more amino acid residues in the Fc region may reduce half-life in systemic circulation (serum), however result in improved retainment in tissues (e.g. in the eye) by disabling FcRn binding (e.g. H435A, EU numbering of Kabat) (Ding et al., 2017, MAbs 9:269-284; and Kim, 1999, Eur J Immunol 29:2819).

In some embodiments, the Fc domain may be engineered to activate all, some, or none of the normal Fc effector functions, without affecting the Fc polypeptide's (e.g. antibody's) desired pharmacokinetic properties. Fc polypeptides having altered effector function may be desirable as they may reduce unwanted side effects, such as activation of effector cells, by the therapeutic protein.

Methods to alter or even ablate effector function may include mutation(s) or modification(s) to the hinge region amino acid residues of an antibody. For example, IgG Fc domain mutants comprising 234A, 237A, and 238S substitutions, according to the EU numbering system, exhibit decreased complement dependent lysis and/or cell mediated destruction. Deletions and/or substitutions in the lower hinge, e.g. where positions 233-236 within a hinge domain (EU numbering) are deleted or modified to glycine, have been shown in the art to significantly reduce ADCC and CDC activity.

In specific embodiments, the Fc domain is an aglycosylated Fc domain that has a substitution at residue 297 or 299 to alter the glycosylation site at 297 such that the Fc domain is not glycosylated. Such aglycosylated Fc domains may have reduced ADCC or other effector activity.

Non-limiting examples of proteins comprising mutant and/or chimeric CH regions having altered effector functions, and methods of engineering and testing mutant antibodies, are described in the art, e.g. K. L. Amour, et al., Eur. J. Immunol. 1999, 29:2613-2624; Lazar et al., Proc. Natl. Acad. Sci. USA 2006, 103:4005; US Patent Application Publication No. 20070135620A1 published Jun. 14, 2007; US Patent Application Publication No. 20080154025 A1, published Jun. 26, 2008; US Patent Application Publication No. 20100234572 A1, published Sep. 16, 2010; US Patent Application Publication No. 20120225058 A1, published Sep. 6, 2012; US Patent Application Publication No. 20150337053 A1, published Nov. 26, 2015; International Publication No. WO20/16161010A2 published Oct. 6, 2016; U.S. Pat. No. 9,359,437, issued Jun. 7, 2016; and U.S. Pat. No. 10,053,517, issued Aug. 21, 2018, all of which are herein incorporated by reference.

The C-terminal lysines (-K) conserved in the heavy chain genes of all human IgG subclasses are generally absent from antibodies circulating in serum—the C-terminal lysines are cleaved off in circulation, resulting in a heterogeneous population of circulating IgGs. (van den Bremer et al., 2015, mAbs 7:672-680). In the vectored constructs for full length mAbs, the DNA encoding the C-terminal lysine (-K) or glycine-lysine (-GK) of the Fc terminus can be deleted to produce a more homogeneous antibody product in situ. (See, Hu et al., 2017 Biotechnol. Prog. 33: 786-794 which is incorporated by reference herein in its entirety).

### 5.2.9 Manufacture and Testing of Vectors

The viral vectors provided herein may be manufactured using host cells. The viral vectors provided herein may be manufactured using mammalian host cells, for example, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (e.g., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

Alternatively, baculovirus expression systems in insect cells may be used to produce AAV vectors. For a review, see Aponte-Ubillus et al., 2018, Appl. Microbiol. Biotechnol. 102:1045-1054 which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Once expressed, characteristics of the expressed product can be determined, including determination of the glycosylation and tyrosine sulfation patterns associated with the human glycosylated anti-TNFα Fc fusion protein. Glycosylation patterns and methods of determining the same are discussed in Section 5.4. In addition, benefits resulting from glycosylation/sulfation of the cell-expressed human glycosylated anti-TNFα Fc fusion proteins can be determined using assays known in the art, e.g., the methods described in Section 5.4.

Vector genome concentration (GC) or vector genome copies can be evaluated using digital PCR (dPCR) or ddPCR™ (BioRad Technologies, Hercules, CA, USA). In one example, ocular tissue samples, such as aqueous and/or vitreous humor samples, are obtained at several timepoints. In another example, several mice are sacrificed at various timepoints post injection. Ocular tissue samples are subjected to total DNA extraction and dPCR assay for vector copy numbers. Copies of vector genome (transgene) per gram of tissue may be measured in a single biopsy sample, or measured in various tissue sections at sequential timepoints will reveal spread of AAV throughout the eye. Total DNA from collected ocular fluid or tissue is extracted with the DNeasy Blood & Tissue Kit and the DNA concentration measured using a Nanodrop spectrophotometer. To determine the vector copy numbers in each tissue sample, digital PCR is performed with Naica Crystal Digital PCR system (Stilla technologies). Two color multiplexing system is applied to simultaneously measure the transgene AAV and an endogenous control gene. In brief, the transgene probe can be labelled with FAM (6-carboxyfluorescein) dye while the endogenous control probe can be labelled with VIC fluorescent dye. The copy number of delivered vector in a specific tissue section per diploid cell is calculated as: (vector copy number)/(endogenous control)×2. Vector copy in specific cell types or tissues, such as cornea, iris, ciliary body, schlemm's canal cells, trabecular meshwork, retinal cells, RPE cells, RPE-choroid tissue, or optic nerve cells, over time may indicate sustained expression of the transgene by the tissue.

### 5.2.9 Compositions

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

### 5.3 Methods of Treating Non-Infectious Uveitis

In another aspect, methods for treating non-infectious uveitis or other indication that can be treated with an anti-TNFα Fc Fusion protein in a subject in need thereof comprising the administration of recombinant AAV particles comprising an expression cassette encoding anti-TNFα Fc Fusion protein and variants thereof, are provided. A subject in need thereof includes a subject suffering from non-infectious uveitis, or a subject pre-disposed thereto, e.g., a subject at risk of developing or having a recurrence of the non-infectious uveitis, or other indication that may be treated with anti-TNFα Fc Fusion protein. Subjects to whom such gene therapy is administered can be those responsive to anti-TNFα, e.g. etanercept, adalimumab, infliximab, or golimumab, In particular embodiments, the methods encompass treating patients who have been diagnosed with non-infectious uveitis, and, in certain embodiments, identified as responsive to treatment with an anti-TNFα Fc Fusion protein or considered a good candidate for therapy with an anti-TNFα Fc Fusion protein. In specific embodiments, the patients have previously been treated with an anti-TNFα Fc Fusion protein. To determine responsiveness, the anti-TNFα Fc Fusion protein (e.g., produced in human cell culture, bioreactors, etc.) may be administered directly to the subject.

In specific embodiments, provided are methods of treating non-infectious uveitis or other indication amenable to treatment with an anti-TNFα Fc Fusion protein in a human subject in need thereof comprising: administering to the eye (or liver and/or muscle) of said subject a therapeutically effective amount of a recombinant nucleotide expression vector comprising a transgene encoding an anti-TNFα Fc Fusion protein an Fc region operably linked to one or more regulatory sequences that control expression of the transgene in human ocular tissue cells, so that a depot is formed that releases a HuPTM form of mAb or antigen-binding fragment thereof. Subretinal, intravitreal, intracamerally, or suprachoroidal administration should result in expression of the soluble transgene product in one or more of the following retinal cell types: human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells or other ocular tissue cell: cornea cells, iris cells, ciliary body cells, a schlemm's canal cells, a trabecular meshwork cells, RPE-choroid tissue cells, or optic nerve cells.

Recombinant vectors and pharmaceutical compositions for treating diseases or disorders in a subject in need thereof are described in Section 5.2. Such vectors should have a tropism for human ocular tissue, or liver and/or muscle cells and can include non-replicating rAAV, particularly those bearing an AAV2.7m8, AAV3B, AAV8, AAAV9, AAV10, AAVrh10, or AAVrh73 capsid. The recombinant vectors can be administered in any manner such that the recombinant vector enters ocular tissue cells, e.g., by introducing the recombinant vector into the eye. Such vectors should further comprise one or more regulatory sequences that control expression of the transgene in human ocular tissue cells and/or human liver and muscle cells include, but are not limited to, human rhodopsin kinase (GRK1) promoter (SEQ ID NOS:54 or 117), a mouse cone arresting (CAR) promoter (SEQ ID NOS:114-116), a human red opsin (RedO) promoter (SEQ ID NO:112) or a Best1/GRK promoter (SEQ ID NO: 161) (see also Tables 1 and 1a).

### 5.4 N-Glycosylation, Tyrosine Sulfation, and O-Glycosylation

The amino acid sequence (primary sequence) of HuPTM anti-TNFα Fc fusion proteins disclosed herein each comprises at least one site at which N-glycosylation or O-glycosylation takes place (see FIGS. 2A and 2B) for glycosylation positions within the amino acid sequences of the Fc fusion proteins). Post-translational modification also occurs in the Fc domain of full length antibodies, particularly at residue N297 (by EU numbering, see FIG. 4).

Alternatively, mutations may be introduced into the Fc domain to alter the glycosylation site at residue N297 (EU numbering, see FIG. 4), in particular substituting another amino acid for the asparagine at 297 or the threonine at 299 to remove the glycosylation site resulting in an aglycosylated Fc domain.

### 5.4.1 N-Glycosylation

### Reverse Glycosylation Sites

The canonical N-glycosylation sequence is known in the art to be Asn-X-Ser (or Thr), wherein X can be any amino acid except Pro. However, it recently has been demonstrated that asparagine (Asn) residues of human antibodies can be glycosylated in the context of a reverse consensus motif, Ser(or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, certain anti-TNFα Fc fusion proteins disclosed herein comprise canonical and/or reverse consensus sequences.

### Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, certain of the HuGlyFab fragments disclosed herein comprise such non-consensus sequences. In addition, O-glycosylation comprises the addition of N-acetyl-galactosamine to serine or threonine residues by the enzyme. It has been demonstrated that amino acid residues present in the hinge region of antibodies can be O-glycosylated. The possibility of O-glycosylation confers another advantage to the therapeutic anti-TNFα Fc fusion proteins provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.)

### Engineered N-Glycosylation Sites

In certain embodiments, a nucleic acid encoding an anti-TNFα Fc Fusion protein is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuPTM anti-TNFα Fc Fusion protein (e.g., relative to the number of N-glycosylation sites associated with the HuPTM anti-TNFα Fc Fusion protein in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antibody or antigen-binding fragment to its antigen. Introduction of glycosylation sites can be accomplished by, e.g., adding new amino acids to the primary structure of the soluble extracellular TNFR-domain or Fc domain (e.g., the glycosylation sites are added, in full or in part), or by mutating existing amino acids in the soluble extracellular TNFR domain or Fc domain, in order to generate the N-glycosylation sites (e.g., amino acids are not added but selected amino acids of the anti-TNFα Fc Fusion protein are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, an anti-TNFα Fc Fusion protein is modified such that, when expressed in mammalian cells, such as retina, CNS, liver or muscle cells, it can be hyperglycosylated.

### N-Glycosylation of HuPTM Anti TNFα Fc Fusion Proteins

Unlike small molecule drugs, biologics usually comprise a mixture of many variants with different modifications or forms that could have a different potency, pharmacokinetics, and/or safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein can be, for example, to slow or arrest the progression of a disease or abnormal condition or to reduce the severity of one or more symptoms associated with the disease or abnormal condition.

When a HuPTM anti-TNFα Fc fusion protein is expressed in a human cell, the N-glycosylation sites of the Fc fusion protein can be glycosylated with various different glycans. Glycosylation of the Fc domain has been characterized and is a single N-linked glycan at asparagine 297 (EU numbering; see FIG. 4). The glycan plays an integral structural and functional role, impacting antibody effector function, such as binding to Fc receptor (see, for example, Jennewein and Alter, 2017, Trends In Immunology 38:358 for a discussion of the role of Fc glycosylation in antibody function). Removal of the Fc region glycan almost completely ablates effector function (Jennewien and Alter at 362). The composition of the Fc glycan has been shown to impact effector function, for example hypergalactosylation and reduction in fucosylation have been shown to increase ADCC activity while sialylation correlates with anti-inflammatory effects (Id. at 364). Disease states, genetics and even diet can impact the composition of the Fc glycan in vivo. For recombinantly expressed antibodies and Fc fusion proteins, the glycan composition can differ significantly by the type of host cell used for recombinant expression and strategies are available to control and modify the composition of the glycan in therapeutic antibodies recombinantly expressed in cell culture, such as CHO to alter effector function (see, for example, US 2014/0193404 by Hansen et al.). Accordingly, the HuPTM anti-TNFα Fc fusion proteins provided herein may advantageously have a glycan at N297 that is more like the native, human glycan composition than antibodies expressed in non-human host cells.

Importantly, when the HuPTM anti-TNFα Fc fusion proteins are expressed in human cells, the need for in vitro production in prokaryotic host cells (e.g., E. coli) or eukaryotic host cells (e.g., CHO cells or NS0 cells) is circumvented. Instead, as a result of the methods described herein, N-glycosylation sites of the HuPTM anti-TNFα Fc fusion proteins are advantageously decorated with glycans relevant to and beneficial to treatment of humans. Such an advantage is unattainable when CHO cells, NS0 cells, or E. coli are utilized in antibody/antigen-binding fragment production, because e.g., CHO cells (1) do not express 2,6 sialyltransferase and thus cannot add 2,6 sialic acid during N-glycosylation; (2) can add Neu5Gc as sialic acid instead of Neu5Ac; and (3) can also produce an immunogenic glycan, the α-Gal antigen, which reacts with anti-α-Gal antibodies present in most individuals, which at high concentrations can trigger anaphylaxis; and because (4) E. coli does not naturally contain components needed for N-glycosylation.

Assays for determining the glycosylation pattern of anti-TNFα Fc fusion proteins are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with anti-TNFα Fc fusion proteins include those described by Montacir et al., 2018, The Protein Journal, 37(2), 164-179.

Homogeneity or heterogeneity of the glycan patterns associated with anti-TNFα Fc fusion proteins, as it relates to both glycan length or size and numbers glycans present across glycosylation sites, can be assessed using methods known in the art, e.g., methods that measure glycan length or size and hydrodynamic radius. HPLC, such as size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

In certain embodiments, the HuPTM anti-TNFα Fc fusion proteins also do not contain detectable NeuGc and/or α-Gal. By “detectable NeuGc” or “detectable α-Gal” or “does not contain or does not have NeuGc or α-Gal” means herein that the HuPTM anti-TNFα Fc fusion protein does not contain NeuGc or α-Gal moieties detectable by standard assay methods known in the art. For example, NeuGc may be detected by HPLC according to Hara et al., 1989, “Highly Sensitive Determination of N-Acetyl- and N-Glycolylneuraminic Acids in Human Serum and Urine and Rat Serum by Reversed-Phase Liquid Chromatography with Fluorescence Detection.” J. Chromatogr., B: Biomed. 377, 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring α-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5):699-710. See also the references cited in Platts-Mills et al., 2015, “Anaphylaxis to the Carbohydrate Side-Chain Alpha-gal” Immunol Allergy Clin North Am. 35(2): 247-260.

### Benefits of N-Glycosylation

N-glycosylation confers numerous benefits on the HuPTM anti-TNFα Fc fusion proteins described herein. Such benefits are unattainable by production of anti-TNFα Fc fusion proteins in E. coli, because E. coli does not naturally possess components needed for N-glycosylation. Further, some benefits are unattainable through antibody production in, e.g., CHO cells (or murine cells such as NS0 cells), because CHO cells lack components needed for addition of certain glycans (e.g., 2,6 sialic acid and bisecting GlcNAc) and because either CHO or murine cell lines add N—N-Glycolylneuraminic acid (“Neu5Gc” or “NeuGc”) which is not natural to humans (and potentially immunogenic), instead of N-Acetylneuraminic acid (“Neu5Ac”) the predominant human sialic acid. See, e.g., Dumont et al., 2015, Crit. Rev. Biotechnol. 36(6):1110-1122; Huang et al., 2006, Anal. Biochem. 349:197-207 (NeuGc is the predominant sialic acid in murine cell lines such as SP2/0 and NS0); and Song et al., 2014, Anal. Chem. 86:5661-5666, each of which is incorporated by reference herein in its entirety). Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat. Biotech. 28:1153-1156. The human glycosylation pattern of the HuPTM anti-TNFα Fc fusion proteins described herein should reduce immunogenicity of the transgene product and improve efficacy.

While non-canonical glycosylation sites usually result in low level glycosylation (e.g., 1-5%) of the anti-TNFα Fc fusion protein population, the functional benefits may be significant (See, e.g., van de Bovenkamp et al., 2016, J. Immunol. 196:1435-1441). For example, glycosylation may affect the stability, half-life, and binding characteristics of an antibody. To determine the effects of anti-TNFα Fc fusion proteins glycosylation on the affinity of the fusion protein for its target, any technique known to one of skill in the art may be used, for example, enzyme linked immunosorbent assay (ELISA), or surface plasmon resonance (SPR). To determine the effects of glycosylation on the half-life of the anti-TNFα Fc fusion proteins, any technique known to one of skill in the art may be used, for example, by measurement of the levels of radioactivity in the blood or organs in a subject to whom a radiolabelled antibody has been administered. To determine the effects of glycosylation on the stability, for example, levels of aggregation or protein unfolding, of the anti-TNFα Fc fusion proteins, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement.

The presence of sialic acid on HuPTM anti-TNFα Fc fusion proteins used in the methods described herein can impact clearance rate of the HuPTM anti-TNFα Fc fusion proteins. Accordingly, sialic acid patterns of a HuPTM anti-TNFα Fc fusion proteins can be used to generate a therapeutic having an optimized clearance rate. Methods of assessing anti-TNFα Fc fusion proteins clearance rate are known in the art.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an anti-TNFα Fc fusion protein used herein or engineered into an antigen-binding fragment used herein, resulting in HuPTM anti-TNFα Fc fusion proteins that is less prone to aggregation when expressed, e.g., expressed in human cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein or engineered into an antigen-binding fragment used herein, resulting in an HuPTM anti-TNFα Fc fusion protein that is less prone to immunogenicity when expressed, e.g., expressed in human ocular tissue cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223-1245.

In another specific embodiment, a benefit conferred by N-glycosylation is altered binding affinity. Assays for measuring binding affinity are known in the art.

### 5.4.2 O-Glycosylation

O-glycosylation comprises the addition of N-acetyl-galactosamine to serine or threonine residues by the enzyme. It has been demonstrated that amino acid residues present in the hinge region of anti-TNFα Fc fusion proteins can be O-glycosylated. In certain embodiments, the anti-TNFα Fc fusion proteins comprise all or a portion of their hinge region, and thus are capable of being O-glycosylated when expressed in human cells. The possibility of O-glycosylation confers another advantage to the anti-TNFα Fc fusion proteins provided herein, as compared to, e.g., anti-TNFα Fc fusion proteins produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.) O-glycosylated anti-TNFα Fc fusion proteins, by virtue of possessing glycans, shares advantageous characteristics with N-glycosylated anti-TNFα Fc fusion proteins (as discussed above).

### 5.5 Vectored HuPTM Anti-TNFα Fc Fusion Protein Constructs and Formulations for Non-Infectious Uveitis

Compositions and methods are described for the delivery of HuPTM anti-TNFα Fc fusion proteins, such as TNFR1:Fc fusion proteins or TNFR2:Fc fusion proteins, that bind to tumor necrosis factor-alpha (TNFα), such as etanercept or EYS606 (FIGS. 2A and 2B) and indicated for treating non-infectious uveitis. In certain embodiments, the HuPTM anti-TNFα Fc fusion protein has the amino acid sequence of etanercept or EYS606, or biosimilars or biobetters thereof. Amino acid sequences of the soluble, extracellular portion of human TNFα receptor type I (TNFR1) or type II (TNFR2) and Fc domains of the fusion proteins are provided in FIGS. 2A and 2B and Table 6. Delivery may be accomplished via gene therapy—e.g., by administering a viral vector or other DNA expression construct encoding an therapeutic anti-TNFα Fc fusion protein (and/or a hyperglycosylated derivative or other derivative, thereof) to patients (human subjects) diagnosed with one or more symptoms of non-infectious uveitis to create a permanent depot that continuously supplies the HuPTM, e.g., human-glycosylated, transgene product to ocular tissues.

### Transgenes

Provided are recombinant vectors containing a transgene encoding a HuPTM anti-TNFα Fc fusion protein that binds to TNFα that can be administered to deliver the HuPTM anti-TNFα Fc fusion protein in a patient. The transgene is a nucleic acid comprising the nucleotide sequences encoding an anti-TNFα Fc fusion protein comprising a soluble, extracellular portion of human TNFα receptor type I (TNFR1) or type II (TNFR2) and a Fc domain, such as etanercept or EYS606, or variants thereof as detailed herein. The transgene may also encode an anti-TNFα Fc fusion protein that contains additional glycosylation sites (e.g., see Courtois et al.).

In certain embodiments, the anti-TNFα transgene comprises the nucleotide sequence encoding the TNFR2:Fc fusion protein etanercept (having amino acid sequences of SEQ ID NO: 10 or 11, see Table 6 and FIG. 2A). The nucleotide sequences may be codon optimized for expression in human cells. Nucleotide sequences may, for example, comprise the nucleotide sequences of SEQ ID NO: 13-18 (encoding the etanercept transgene sequences as set forth in Table 7. The TNFR2:Fc fusion sequences has a signal or leader sequence at the N-terminus appropriate for expression and secretion in human cells, in particular, one or more cells ocular tissue cells. The signal sequence may have the amino acid sequence of MYRMQLLLLIALSLALVTNS (SEQ ID NO:62). Alternatively, the signal sequence may have an amino acid sequence selected from any one of the signal sequences set forth in Table 2 that correspond to the proteins secreted by one or more ocular tissue cells. Alternatively, the signal sequence may be appropriate for expression in muscle or liver cells, such as those listed in Tables 3 and 4 infra.

In addition to the soluble, extracellular protein of the TNFR type 2 domain and a Fc constant region including C_{H}2 and C_{H}3 domain sequences, the transgene may comprise, at the N-terminus of the heavy chain C_{H}2 domain sequence, all or a portion of the hinge region. In specific embodiments, the Fc domain has an amino acid sequence of SEQ ID NO: 4 with an additional hinge region sequence at the N-terminus (e.g. SEQ ID NO: 3 with hinge) or containing all or a portion of the amino acid sequence EPKSCDKTHTCPPCPAPELLGG (SEQ ID NO:145), and specifically, EPKSCDKTHL (SEQ ID NO:146), EPKSCDKTHT (SEQ ID NO:147), EPKSCDKTHTCPPCPA (SEQ ID NO:148), EPKSCDKTHLCPPCPA (SEQ ID NO:149), EPKSCDKTHTCPPCPAPELLGGPSVFL (SEQ ID NO:150) or EPKSCDKTHLCPPCPAPELLGGPSVFL (SEQ ID NO:151). The Fc domain of the anti-TNFα Fc fusion protein may have an amino acid sequence of SEQ ID NO: 3 or 4 (Table 6) or an IgG1 Fc domain, such as depicted in FIG. 4, or a mutant or variant thereof. The Fc domain may be engineered for altered binding to one or more Fc receptors and/or effector function as disclosed in Section 5.2.8, infra.

Expression of the etanercept may be directed by a constitutive or a tissue specific promoter. In certain embodiments, the transgene contains a CAG promoter (SEQ ID NO:51), CB promoter (SEQ ID NO: 159), CBLong promoter (SEQ ID NO: 161), a GRK1 (SEQ ID NO:54) promoter or a Best1/GRK promoter (SEQ ID NO: 161). Alternatively, the promoter may be a tissue specific promoter (or regulatory sequence including promoter and enhancer elements) such as the GRK1 promoter (SEQ ID NO:54 or 117), (a mouse cone arresting (CAR) promoter (SEQ ID NOS:114-116), a human red opsin (RedO) promoter (SEQ ID NO:112) or a Best1/GRK promoter (SEQ ID NO: 161). See FIG. 1 for a schematic showing the genomic configuration. The transgenes may contain elements provided in Table 1. Exemplary transgenes encoding etanercept are provided in Table 7 and include CAG.etanercept (SEQ ID NO: 16) or mU1a.Vh4i.etanercept.scAAV (SEQ ID NO: 18). ITR sequences are added to the 5′ and 3; ends of the constructs to generate the genomes resulting in CAG.etanercept (SEQ ID NO: 15) and Ula.Vh4i.etanercept.scAAV (SEQ ID NO: 17). In certain embodiments, the construct is a self-complementary construct. The transgenes may be packaged into AAV, particularly AAV2.7m8, AAV8, AAV3B, or AAVrh73.

In certain embodiments, the transgene encodes an anti-TNFα Fc fusion protein comprising a soluble, extracellular portion of the TNFR type 2 domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the transgene encodes an anti-TNFα Fc fusion protein comprising a Fc domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, transgene encodes an anti-TNFα Fc fusion protein comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 10 or 11. In specific embodiments, the TNFR2:Fc fusion protein comprising an amino acid sequence of SEQ ID NO: 10 or 11 with 1, 2, 3, 4, 5, 6, 7, 8, 9, 10, 11, 12, 13, 14, 15 or more amino acid substitutions, insertions or deletions, and the substitutions, insertions or deletions are made.

In certain embodiments, the anti-TNFα transgene comprises the nucleotide sequence encoding the TNFR1:Fc fusion protein EYS606 (having amino acid sequence of SEQ ID NO: 12, see Table 6 and FIG. 2B). The nucleotide sequences may be codon optimized for expression in human cells. The TNFR1:Fc fusion protein has a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO:62). Alternatively, the signal sequence may have an amino acid sequence selected from any one of the signal sequences set forth in Table 2 that correspond to the proteins secreted by one or more ocular tissue cells. Alternatively, the signal sequence may be appropriate for expression in muscle or liver cells, such as those listed in Tables 3 and 4 infra.

In addition to the soluble, extracellular protein of the TNFR type 1 domain and a Fc constant region including C_{H}2 and C_{H}3 domain sequences, the transgene may comprise, at the N-terminus of the heavy chain C_{H}2 domain sequence, a thrombin cleavage site LVPRGS (SEQ ID NO:8) and all or a portion of the hinge region. In specific embodiments, the Fc domain has an amino acid sequence of SEQ ID NO: 3 or has a hinge region sequence at the N-terminus containing all or a portion of the amino acid sequence DKTHTCPPCPAPELLGG (SEQ ID NO:21), and specifically, DKTHTCPPCPA (SEQ ID NO:154), DKTHLCPPCPA (SEQ ID NO:155), DKTHTCPPCPAPELLGGPSVFL (SEQ ID NO:152) or DKTHLCPPCPAPELLGGPSVFL (SEQ ID NO:156). The Fc domain of the anti-TNFα Fc fusion protein may have an amino acid sequence of SEQ ID NO: 10 (Table 6). The Fc domain may be engineered for altered binding to one or more Fc receptors and/or effector function as disclosed in Section 5.2.8, infra.

Expression of EYS606 may be directed by a constitutive or a tissue specific promoter. In certain embodiments, the transgene contains a CAG promoter (SEQ ID NO:51) or a GRK1 (SEQ ID NO:54) promoter. Alternatively, the promoter may be a tissue specific promoter (or regulatory sequence including promoter and enhancer elements) such as the GRK1 promoter (SEQ ID NO:54 or 117), (a mouse cone arresting (CAR) promoter (SEQ ID NOS:114-116), a human red opsin (RedO) promoter (SEQ ID NOl12) or a Best1/GRK promoter (SEQ ID NO:161). The transgenes may contain elements provided in Table 1. Exemplary transgenes encoding EYS606 may be generated using methods known in the art. ITR sequences are added to the 5′ and 3; ends of the constructs to generate the genomes. The transgenes may be packaged into AAV, particularly AAV8, AAV3B, or AAVrh73.

In certain embodiments, the transgene encodes an anti-TNFα Fc fusion protein comprising a soluble, extracellular portion of the TNFR type 1 domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the transgene encodes an anti-TNFα Fc fusion protein comprising a Fc domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 6. In certain embodiments, transgene encodes an anti-TNFα Fc fusion protein comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 12. In specific embodiments, the TNFR1:Fc fusion protein comprises a soluble, extracellular portion of the TNFR type 1 domain comprising an amino acid sequence of SEQ ID NO: 1 with 1, 2, 3, 4, 5, 6, 7, 8, 9, 10, 11, 12, 13, 14, 15 or more amino acid substitutions, insertions or deletions, and the substitutions, insertions or deletions are made. In specific embodiments, the TNFR1:Fc fusion protein comprises an amino acid sequence of SEQ ID NO: 12 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions.

### Gene Therapy Methods

Provided are methods of treating human subjects for non-infectious uveitis by administration of a viral vector containing a transgene encoding an anti-TNFα fusion protein. The fusion protein may be etanercept or EYS606, or a biobetter or biosimilar thereof. In embodiments, the patient has been diagnosed with and/or has symptom(s) associated with non-infectious uveitis. Recombinant vector used for delivering the transgene are described in Section 5.2. Such vectors should have a tropism for human ocular tissue cells and can include non-replicating rAAV, particularly those bearing an AAV8 capsid, an AAV3B capsid, or an AAVr73 capsid. Alternatively, vectors bearing an AAV2.7m8 or AAV9 capsid can be used for ocular indications. The recombinant vector, such as the one shown in FIG. 1, can be administered in any manner such that the recombinant vector enters one or more ocular tissue cell types, e.g. by introducing the recombinant vector into the eye. See Section 5.3 for details regarding the methods of treatment.

Provided are methods of treating human subjects for non-infectious uveitis by administration of a viral vector containing a transgene encoding an anti-TNFα fusion protein. The fusion protein may be etanercept or EYS606. In embodiments, the patient has been diagnosed with and/or has symptoms associated with non-infectious uveitis. Recombinant vectors used for delivering the transgene are described in Section 5.2 and exemplary transgenes are provided above. Such vectors should have a tropism for human ocular tissue cells and can include non-replicating rAAV, particularly those bearing an AAV3B, AAV8, or AAVrh73 capsid. The recombinant vectors, such as shown in FIGS. 2A and 2B, can be administered in any manner such that the recombinant vector enters the ocular tissue. In particular embodiments, the transgene is SEQ ID NO: 13-18 in an AAV8 vector (see Table 7).

Subjects to whom such gene therapy is administered can be those responsive to anti-TNFα therapy. In certain embodiments, the methods encompass treating patients who have been diagnosed with non-infectious uveitis, or have one or more symptoms associated therewith, and identified as responsive to treatment with an anti-TNFα antibody, anti-TNFα Fc fusion protein, or considered a good candidate for therapy with an anti-TNFα antibody or anti-TNFα Fc fusion protein. In specific embodiments, the patients have previously been treated with etanercept, adalimumab, infliximab, or golimumab, and have been found to be responsive to etanercept, adalimumab, infliximab, or golimumab. In other embodiments, the patients have been previously treated with an anti-TNF-alpha antibody or fusion protein such as etanercept, certolizumab, or other anti-TNF-alpha agent. To determine responsiveness, the anti-TNFα transgene product (e.g., produced in cell culture, bioreactors, etc.) may be administered directly to the subject.

### Human Post Translationally Modified Anti-TNFα Fusion Proteins

The production of the HuPTM anti-TNFα fusion protein, should result in a “biobetter” molecule for the treatment of non-infectious uveitis accomplished via gene therapy—e.g., by administering a viral vector or other DNA expression construct encoding the anti-TNFα HuPTM anti-TNFα fusion protein, subretinally, intravitreally, intranasally, intracamerally, suprachoroidally, or systemically to human subjects (patients) diagnosed with or having one or more symptoms of non-infectious uveitis, to create a permanent depot in one or more ocular tissues (or cell types) that continuously supplies the fully-human post-translationally modified, e.g., human-glycosylated, sulfated transgene product produced by transduced ocular tissue cells.

In specific embodiments, the HuPTM anti-TNFα fusion protein has a soluble, extracellular portion of the TNFR2 domain fused to a Fc domain with the amino acid sequences of etanercept as set forth in FIG. 2A (asparaginal (N) glycosylation sites, non-consensus asparaginal (N) glycosylation sites; are as indicated in the legend) has a glycosylation, particularly a 2,6-sialylation, at one or more of the amino acid positions indicated in FIG. 2A, including N149 or N171 of the TNFR2 domain (SEQ ID NO: 2) and/or N317 of the Fc domain (SEQ ID NO: 3), and/or O-linked glycosylation sites at one or more of the amino acid positions T8, T184, S199, and/or T200 of the TNFR2 domain (SEQ ID NO: 2) and/or T245 of the hinge region (SEQ ID NO: 11). In other embodiments, the HuPTM anti-TNFα fusion protein does not contain any detectable NeuGc moieties and/or does not contain any detectable alpha-Gal moieties.

In specific embodiments, the HuPTM anti-TNFα fusion protein has a soluble, extracellular portion of the TNFR1 domain fused to a Fc domain with the amino acid sequences of ESY606 as set forth in FIG. 2B (asparaginal (N) glycosylation sites, non-consensus asparaginal (N) glycosylation sites; are as indicated in the legend) has a glycosylation, particularly a 2,6-sialylation, at one or more of the amino acid positions as indicated in FIG. 2B, including N54, N94 or N145 of the TNFR1 domain (SEQ ID NO: 1) and/or N294 or N358 of the Fc domain (SEQ ID NO: 12), and/or O-linked glycosylation sites at one or more of the amino acid positions T8, T184, S199, and/or T200 of the TNFR2 domain (SEQ ID NO: 1) and/or T245 of the hinge region (SEQ ID NO: 12). In other embodiments, the HuPTM anti-TNFα fusion protein does not contain any detectable NeuGc moieties and/or does not contain any detectable alpha-Gal moieties.

In certain embodiments, the HuPTM anti-TNFα Fc fusion protein is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of or relieve one or more symptoms of non-infectious uveitis, such as to reduce the levels of pain, redness of the eye, sensitivity to light, and/or other discomfort for the patient. Efficacy may be monitored by measuring a reduction in pain, redness of the eye, and/or photophobia and/or an improvement in vision.

Combinations of delivery of the HuPTM anti-TNFα˜ Fe fusion protein to the eye accompanied by delivery of other available treatments are encompassed by the methods provided herein. The additional treatments may be administered before, concurrently, or subsequent to the gene therapy treatment. Available treatments for a subject with non-infectious uveitis that could be combined with the gene therapy provided herein include but are not limited to, azathioprine, methotrexate, mycophenolate mofetil, cyclosporine, cyclophosphamide, corticosteroids (local and/or systemic), and others and administration with anti-TNFα agents, including but not limited to etanercept, EYS606, adalimumab, infliximab, or golimumab.

TABLE 6
Anti-TNFα Fc Fusion Amino Acid Sequences

| | SEQ | |
|---|---|---|
| Domain | ID NO. | Sequence |
| TNFR1 | SEQ ID | MGLSTVPDLLLPLVLLELLVGIYPSGVIGLVPHLGDREKRDSV |
| Human | NO: 1 | CPQGKYIHPQNNSICCTKCHKGTYLYNDCPGPGQDTDCRECES |
| TNFRSF1A | | GSFTASENHLRHCLSCSKCRKEMGQVEISSCTVDRDTVCGCRK |
| Extracellular | | NQYRHYWSENLFQCFNCSLCLNGTVHLSCQEKQNTVCTCHAGF |
| domain, | | FLRENECVSCSNCKKSLECTKLCLPQIENVKGTEDSGTT |
| UniProtKB- | | |
| P19438 amino | | |
| acid 1-211 | | |
| TNFR2 | SEQ ID | LPAQVAFTPY APEPGSTCRL REYYDQTAQM CCSKCSPGQH |
| Human | NO: 2 | AKVFCTKTSD TVCDSCEDST YTQLWNWVPE CLSCGSRCSS |
| TNFRSF1B | | DQVETQACTR EQNRICTCRP GWYCALSKQE GCRLCAPLRK |
| Extracellular | | CRPGFGVARP GTETSDVVCK PCAPGTESNT TSSTDICRPH |
| domain, | | QICNVVAIPG NASMDAVCTS TSPTRSMAPG AVHLPQPVST |
| UniProtKB- | | RSQHTQPTPE PSTAPSTSFL LPMGPSPPAE GSTGD |
| P20333 amino | | |
| acid 23-257 | | |
| Etanercept: | SEQ ID | EPKSCDKTHT CPPCPAPELL GGPSVFLFPP KPKDTLMISR |
| Fc (including | NO: 3 | TPEVTCVVVD VSHEDPEVKF NWYVDGVEVH NAKTKPREEQ |
| hinge) | | YNSTYRVVSV LTVLHQDWLN GKEYKCKVSN KALPAPIEKT |
| | | ISKAKGQPRE PQVYTLPPSR EEMTKNQVSL TCLVKGFYPS |
| | | DIAVEWESNG QPENNYKTTP PVLDSDGSFF LYSKLTVDKS |
| | | RWQQGNVFSC SVMHEALHNH YTQKSLSLSP GK |
| Etanercept: | SEQ ID | FLFPPKPKDT LMISRTPEVT CVVVDVSHED PEVKENWYVD |
| Fc (without | NO: 4 | GVEVHNAKTK PREEQYNSTY RVVSVLTVLH QDWLNGKEYK |
| hinge) | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPGK |
| Etanercept: | SEQ ID | EPKSCDKTHTCPPCPAPELLGGPSV |
| Hinge region | NO: 5 | |
| EYS606: Fc | SEQ ID | DKTHTCPPCPAPELLGGPSVFLFPPKPKDTLMISRTPEVTCVV |
| (including | NO: 6 | VDVSHEDPEVKENWYVDGVEVHNAKTKPREEQYNSTYRVVSVL |
| hinge) | | TVLHQDWLNGKEYKCKVSNKALPAPIEKTISKAKGQPREPQVY |
| | | TLPPSREEMTKNQVSLTCLVKGFYPSDIAVEWESNGQPENNYK |
| | | TTPPVLDSDGS |
| EYS606: Fc | SEQ ID | FLFPPKPKDTLMISRTPEVTCVVVDVSHEDPEVKENWYVDGVE |
| (without | NO: 7 | VHNAKTKPREEQYNSTYRVVSVLTVLHQDWLNGKEYKCKVSNK |
| hinge) | | ALPAPIEKTISKAKGQPREPQVYTLPPSREEMTKNQVSLTCLV |
| | | KGFYPSDIAVEWESNGQPENNYKTTPPVLDSDGS |
| EYS606: | SEQ ID | LVPRGS |
| Thrombin | NO: 8 | |
| cleavage | | |
| site | | |
| EYS606: | SEQ ID | DKTHTCPPCPAPELLGGPSV |
| Hinge region | NO: 9 | |
| Etanercept | SEQ ID | LPAQVAFTPY APEPGSTCRL REYYDQTAQM CCSKCSPGQH |
| TNFR2: Fc | NO: 10 | AKVFCTKTSD TVCDSCEDST YTQLWNWVPE CLSCGSRCSS |
| fusion | | DQVETQACTR EQNRICTCRP GWYCALSKQE GCRLCAPLRK |
| | | CRPGFGVARP GTETSDVVCK PCAPGTFSNT TSSTDICRPH |
| | | QICNVVAIPG NASMDAVCTS TSPTRSMAPG AVHLPQPVST |
| | | RSQHTQPTPE PSTAPSTSFL LPMGPSPPAE GSTGDEPKSC |
| | | DKTHTCPPCP APELLGGPSV FLFPPKPKDT LMISRTPEVT |
| | | CVVVDVSHED PEVKFNWYVD GVEVHNAKTK PREEQYNSTY |
| | | RVVSVLTVLH QDWLNGKEYK CKVSNKALPA PIEKTISKAK |
| | | GQPREPQVYT LPPSREEMTK NQVSLTCLVK GFYPSDIAVE |
| | | WESNGQPENN YKTTPPVLDS DGSFFLYSKL TVDKSRWQQG |
| | | NVFSCSVMHE ALHNHYTQKS LSLSPGK |
| Etanercept plus leader sequence | SEQ ID NO: 11 | 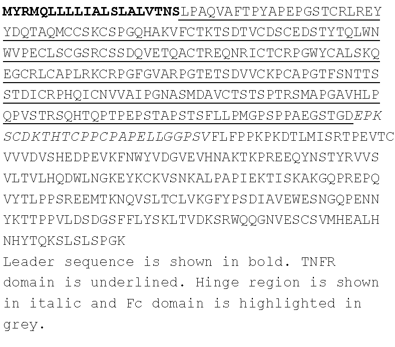 |
| EYS606 | SEQ ID NO: 12 | 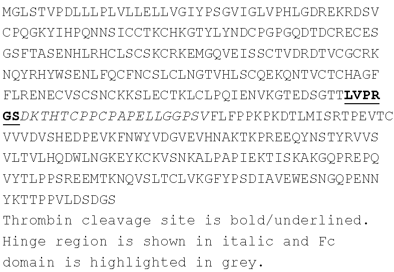 |

TABLE 7
Anti-TNFα Fc Fusion Nucleotide Sequences

| | SEQ | |
|---|---|---|
| Protein | ID NO. | Sequence |
| Etanercept | SEQ ID NO: 13 | 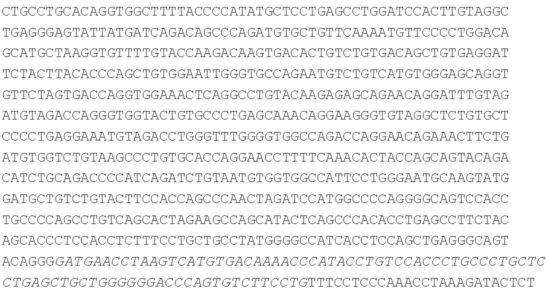 |
| | | GATGATCAGCAGAACCCCAGAGGTCACATGCGTGGTAGTGGATGTCTCCCATGAAGAC |
| | | CCTGAAGTGAAGTTTAATTGGTATGTAGATGGGGTGGAGGTCCATAATGCAAAGACCA |
| | | AGCCCAGGGAAGAACAGTATAACAGTACTTACAGGGTGGTGTCTGTGCTGACAGTACT |
| | | GCACCAGGACTGGCTGAATGGGAAGGAGTATAAGTGCAAAGTCAGCAATAAGGCACTG |
| | | CCTGCCCCTATTGAAAAGACAATTAGTAAAGCTAAGGGGCAGCCCAGGGAACCACAGG |
| | | TCTACACTCTGCCCCCTAGTAGAGAGGAAATGACAAAGAATCAGGTCAGTCTGACATG |
| | | TCTGGTCAAGGGATTCTATCCAAGTGATATTGCAGTGGAGTGGGAAAGTAATGGCCAG |
| | | CCAGAGAATAACTACAAGACTACCCCCCCAGTGCTGGACAGTGATGGATCTTTCTTTC |
| | | TGTACTCCAAACTGACTGTGGATAAATCAAGATGGCAGCAGGGCAATGTCTTTTCCTG |
| | | TTCAGTCATGCATGAAGCCCTGCATAATCACTACACTCAGAAGAGCCTGTCCCTGTCC |
| | | CCTGGCAAATGA |
| | | TNFR2 domain is highlighted in grey. Hinge region is shown |
| | | in italic. |
| Etanercept | SEQ ID | ATGTATAGGATGCAGCTGCTGCTGCTGATTGCTCTGAGTCTGGCTCTGGTCACAAATA |
| plus leader | NO: 14 | GTCTGCCTGCACAGGTGGCTTTTACCCCATATGCTCCTGAGCCTGGATCCACTTGTAG |
| sequence | | GCTGAGGGAGTATTATGATCAGACAGCCCAGATGTGCTGTTCAAAATGTTCCCCTGGA |
| | | CAGCATGCTAAGGTGTTTTGTACCAAGACAAGTGACACTGTCTGTGACAGCTGTGAGG |
| | | ATTCTACTTACACCCAGCTGTGGAATTGGGTGCCAGAATGTCTGTCATGTGGGAGCAG |
| | | GTGTTCTAGTGACCAGGTGGAAACTCAGGCCTGTACAAGAGAGCAGAACAGGATTTGT |
| | | ACATGTAGACCAGGGTGGTACTGTGCCCTGAGCAAACAGGAAGGGTGTAGGCTCTGTG |
| | | CTCCCCTGAGGAAATGTAGACCTGGGTTTGGGGTGGCCAGACCAGGAACAGAAACTTC |
| | | TGATGTGGTCTGTAAGCCCTGTGCACCAGGAACCTTTTCAAACACTACCAGCAGTACA |
| | | GACATCTGCAGACCCCATCAGATCTGTAATGTGGTGGCCATTCCTGGGAATGCAAGTA |
| | | TGGATGCTGTCTGTACTTCCACCAGCCCAACTAGATCCATGGCCCCAGGGGCAGTCCA |
| | | CCTGCCCCAGCCTGTCAGCACTAGAAGCCAGCATACTCAGCCCACACCTGAGCCTTCT |
| | | ACAGCACCCTCCACCTCTTTCCTGCTGCCTATGGGGCCATCACCTCCAGCTGAGGGCA |
| | | GTACAGGGGATGAACCTAAGTCATGTGACAAAACCCATACCTGTCCACCCTGCCCTGC |
| | | TCCTGAGCTGCTGGGGGGACCCAGTGTCTTCCTGTTTCCTCCCAAACCTAAAGATACT |
| | | CTGATGATCAGCAGAACCCCAGAGGTCACATGCGTGGTAGTGGATGTCTCCCATGAAG |
| | | ACCCTGAAGTGAAGTTTAATTGGTATGTAGATGGGGTGGAGGTCCATAATGCAAAGAC |
| | | CAAGCCCAGGGAAGAACAGTATAACAGTACTTACAGGGTGGTGTCTGTGCTGACAGTA |
| | | CTGCACCAGGACTGGCTGAATGGGAAGGAGTATAAGTGCAAAGTCAGCAATAAGGCAC |
| | | TGCCTGCCCCTATTGAAAAGACAATTAGTAAAGCTAAGGGGCAGCCCAGGGAACCACA |
| | | GGTCTACACTCTGCCCCCTAGTAGAGAGGAAATGACAAAGAATCAGGTCAGTCTGACA |
| | | TGTCTGGTCAAGGGATTCTATCCAAGTGATATTGCAGTGGAGTGGGAAAGTAATGGCC |
| | | AGCCAGAGAATAACTACAAGACTACCCCCCCAGTGCTGGACAGTGATGGATCTTTCTT |
| | | TCTGTACTCCAAACTGACTGTGGATAAATCAAGATGGCAGCAGGGCAATGTCTTTTCC |
| | | TGTTCAGTCATGCATGAAGCCCTGCATAATCACTACACTCAGAAGAGCCTGTCCCTGT |
| | | CCCCTGGCAAATGA |
| | | Leader sequence is shown in bold |
| pAAV.CAG. | SEQ ID | ctgcgcgctcgctcgctcactgaggccgcccgggcaaagcccgggcgtcgggcgacct |
| Etanercept | NO: 15 | ttggtcgcccggcctcagtgagcgagcgagcgcgcagagagggagtggccaactccat |
| (from 5'-ITR | | cactaggggttccttgtagttaatgattaacccgccatgctacttatctaccagggta |
| to 3-ITR) | | atggggatcctctagaactatagctagtcgacattgattattgactagttattaatag |
| | | taatcaattacggggtcattagttcatagcccatatatggagttccgcgttacataac |
| | | ttacggtaaatggcccgcctggctgaccgcccaacgacccccgcccattgacgtcaat |
| | | aatgacgtatgttcccatagtaacgccaatagggactttccattgacgtcaatgggtg |
| | | gagtatttacggtaaactgcccacttggcagtacatcaagtgtatcatatgccaagta |
| | | cgccccctattgacgtcaatgacggtaaatggcccgcctggcattatgcccagtacat |
| | | gaccttatgggactttcctacttggcagtacatctacgtattagtcatcgctattacc |
| | | atggtcgaggtgagccccacgttctgcttcactctccccatctcccccccctccccac |
| | | ccccaattttgtatttatttattttttaattattttgtgcagcgatgggggcgggggg |
| | | ggggggggggcgcgcgccaggcggggcggggcggggcgaggggcggggcggggcgagg |
| | | cggagaggtgcggcggcagccaatcagagcggcgcgctccgaaagtttccttttatgg |
| | | cgaggcggcggcggcggcggccctataaaaagcgaagcgcgcggcgggcgggagtcgc |
| | | tgcgcgctgccttcgccccgtgccccgctccgccgccgcctcgcgccgcccgccccgg |
| | | ctctgactgaccgcgttactcccacaggtgagcgggcgggacggcccttctcctccgg |
| | | gctgtaattagcgcttggtttaatgacggcttgtttcttttctgtggctgcgtgaaag |
| | | ccttgaggggctccgggagggccctttgtgcggggggagcggctcggggggtgcgtgc |
| | | gtgtgtgtgtgcgtggggagcgccgcgtgcggctccgcgctgcccggcggctgtgagc |
| | | gctgcgggcgcggcgcggggctttgtgcgctccgcagtgtgcgcgaggggagcgcggc |
| | | cgggggcggtgccccgcggtgcggggggggctgcgaggggaacaaaggctgcgtgcgg |
| | | ggtgtgtgcgtgggggggtgagcagggggtgtgggcgcgtcggtcgggctgcaacccc |
| | | ccctgcacccccctccccgagttgctgagcacggcccggcttcgggtgcggggctccg |
| | | tacggggcgtggcgcggggctcgccgtgccgggcggggggtggcggcaggtgggggtg |
| | | ccgggcggggcggggccgcctcgggccggggagggctcgggggaggggcgcggcggcc |
| | | cccggagcgccggcggctgtcgaggcgcggcgagccgcagccattgccttttatggta |
| | | atcgtgcgagagggcgcagggacttcctttgtcccaaatctgtgcggagccgaaatct |
| | | gggaggcgccgccgcaccccctctagcgggcgcggggcgaagcggtgcggcgccggca |
| | | ggaaggaaatgggcggggagggccttcgtgcgtcgccgcgccgccgtccccttctccc |
| | | tctccagcctcggggctgtccgcggggggacggctgccttcgggggggacggggcagg |
| | | gcggggttcggcttctggcgtgtgaccggcggctctagagcctctgctaaccatgttc |
| | | atgccttcttctttttcctacagctcctgggcaacgtgctggttattgtgctgtctca |
| | | tcattttggcaaagAATTCGCCACCATGTATAGGATGCAGCTGCTGCTGCTGATTGCT |
| | | CTGAGTCTGGCTCTGGTCACAAATAGTCTGCCTGCACAGGTGGCTTTTACCCCATATG |
| | | CTCCTGAGCCTGGATCCACTTGTAGGCTGAGGGAGTATTATGATCAGACAGCCCAGAT |
| | | GTGCTGTTCAAAATGTTCCCCTGGACAGCATGCTAAGGTGTTTTGTACCAAGACAAGT |
| | | GACACTGTCTGTGACAGCTGTGAGGATTCTACTTACACCCAGCTGTGGAATTGGGTGC |
| | | CAGAATGTCTGTCATGTGGGAGCAGGTGTTCTAGTGACCAGGTGGAAACTCAGGCCTG |
| | | TACAAGAGAGCAGAACAGGATTTGTACATGTAGACCAGGGTGGTACTGTGCCCTGAGC |
| | | AAACAGGAAGGGTGTAGGCTCTGTGCTCCCCTGAGGAAATGTAGACCTGGGTTTGGGG |
| | | TGGCCAGACCAGGAACAGAAACTTCTGATGTGGTCTGTAAGCCCTGTGCACCAGGAAC |
| | | CTTTTCAAACACTACCAGCAGTACAGACATCTGCAGACCCCATCAGATCTGTAATGTG |
| | | GTGGCCATTCCTGGGAATGCAAGTATGGATGCTGTCTGTACTTCCACCAGCCCAACTA |
| | | GATCCATGGCCCCAGGGGCAGTCCACCTGCCCCAGCCTGTCAGCACTAGAAGCCAGCA |
| | | TACTCAGCCCACACCTGAGCCTTCTACAGCACCCTCCACCTCTTTCCTGCTGCCTATG |
| | | GGGCCATCACCTCCAGCTGAGGGCAGTACAGGGGATGAACCTAAGTCATGTGACAAAA |
| | | CCCATACCTGTCCACCCTGCCCTGCTCCTGAGCTGCTGGGGGGACCCAGTGTCTTCCT |
| | | GTTTCCTCCCAAACCTAAAGATACTCTGATGATCAGCAGAACCCCAGAGGTCACATGC |
| | | GTGGTAGTGGATGTCTCCCATGAAGACCCTGAAGTGAAGTTTAATTGGTATGTAGATG |
| | | GGGTGGAGGTCCATAATGCAAAGACCAAGCCCAGGGAAGAACAGTATAACAGTACTTA |
| | | CAGGGTGGTGTCTGTGCTGACAGTACTGCACCAGGACTGGCTGAATGGGAAGGAGTAT |
| | | AAGTGCAAAGTCAGCAATAAGGCACTGCCTGCCCCTATTGAAAAGACAATTAGTAAAG |
| | | CTAAGGGGCAGCCCAGGGAACCACAGGTCTACACTCTGCCCCCTAGTAGAGAGGAAAT |
| | | GACAAAGAATCAGGTCAGTCTGACATGTCTGGTCAAGGGATTCTATCCAAGTGATATT |
| | | GCAGTGGAGTGGGAAAGTAATGGCCAGCCAGAGAATAACTACAAGACTACCCCCCCAG |
| | | TGCTGGACAGTGATGGATCTTTCTTTCTGTACTCCAAACTGACTGTGGATAAATCAAG |
| | | ATGGCAGCAGGGCAATGTCTTTTCCTGTTCAGTCATGCATGAAGCCCTGCATAATCAC |
| | | TACACTCAGAAGAGCCTGTCCCTGTCCCCTGGCAAATGATAACtcgaggacggggtga |
| | | actacgcctgaggatccgatctttttccctctgccaaaaattatggggacatcatgaa |
| | | gccccttgagcatctgacttctggctaataaaggaaatttattttcattgcaatagtg |
| | | tgttggaattttttgtgtctctcactcggaagcaattcgttgatctgaatttcgacca |
| | | cccataatacccattaccctggtagataagtagcatggcgggttaatcattaactaca |
| | | aggaacccctagtgatggagttggccactccctctctgcgcgctcgctcgctcactga |
| | | ggccgggcgaccaaaggtcgcccgacgcccgggctttgcccgggcggcctcagtgagc |
| | | gagcgagcgcgcag |
| pAAV.CAG. | SEQ ID | acattgattattgactagttattaatagtaatcaattacggggtcattagttcatagc |
| Etanercept | NO: 16 | ccatatatggagttccgcgttacataacttacggtaaatggcccgcctggctgaccgc |
| (from | | ccaacgacccccgcccattgacgtcaataatgacgtatgttcccatagtaacgccaat |
| promoter to | | agggactttccattgacgtcaatgggtggagtatttacggtaaactgcccacttggca |
| polyA) | | gtacatcaagtgtatcatatgccaagtacgccccctattgacgtcaatgacggtaaat |
| | | ggcccgcctggcattatgcccagtacatgaccttatgggactttcctacttggcagta |
| | | catctacgtattagtcatcgctattaccatggtcgaggtgagccccacgttctgcttc |
| | | actctccccatctcccccccctccccacccccaattttgtatttatttattttttaat |
| | | tattttgtgcagcgatgggggcggggggggggggggggcgcgcgccaggcggggcggg |
| | | gcggggcgaggggcggggcggggcgaggcggagaggtgcggcggcagccaatcagagc |
| | | ggcgcgctccgaaagtttccttttatggcgaggcggcggcggcggcggccctataaaa |
| | | agcgaagcgcgcggcgggcgggagtcgctgcgcgctgccttcgccccgtgccccgctc |
| | | cgccgccgcctcgcgccgcccgccccggctctgactgaccgcgttactcccacaggtg |
| | | agcgggcgggacggcccttctcctccgggctgtaattagcgcttggtttaatgacggc |
| | | ttgtttcttttctgtggctgcgtgaaagccttgaggggctccgggagggccctttgtg |
| | | cggggggagcggctcggggggtgcgtgcgtgtgtgtgtgcgtggggagcgccgcgtgc |
| | | ggctccgcgctgcccggcggctgtgagcgctgcgggcgcggcgcggggctttgtgcgc |
| | | tccgcagtgtgcgcgaggggagcgcggccgggggcggtgccccgcggtgcgggggggg |
| | | ctgcgaggggaacaaaggctgcgtgcggggtgtgtgcgtgggggggtgagcagggggt |
| | | gtgggcgcgtcggtcgggctgcaaccccccctgcacccccctccccgagttgctgagc |
| | | acggcccggcttcgggtgcggggctccgtacggggcgtggcgcggggctcgccgtgcc |
| | | gggcggggggtggcggcaggtgggggtgccgggcggggcggggccgcctcgggccggg |
| | | gagggctcgggggaggggcgcggcggcccccggagcgccggcggctgtcgaggcgcgg |
| | | cgagccgcagccattgccttttatggtaatcgtgcgagagggcgcagggacttccttt |
| | | gtcccaaatctgtgcggagccgaaatctgggaggcgccgccgcaccccctctagcggg |
| | | cgcggggcgaagcggtgcggcgccggcaggaaggaaatgggcggggagggccttcgtg |
| | | cgtcgccgcgccgccgtccccttctccctctccagcctcggggctgtccgcgggggga |
| | | cggctgccttcgggggggacggggcagggcggggttcggcttctggcgtgtgaccggc |
| | | ggctctagagcctctgctaaccatgttcatgccttcttctttttcctacagctcctgg |
| | | gcaacgtgctggttattgtgctgtctcatcattttggcaaagAATTCGCCACCATGTA |
| | | TAGGATGCAGCTGCTGCTGCTGATTGCTCTGAGTCTGGCTCTGGTCACAAATAGTCTG |
| | | CCTGCACAGGTGGCTTTTACCCCATATGCTCCTGAGCCTGGATCCACTTGTAGGCTGA |
| | | GGGAGTATTATGATCAGACAGCCCAGATGTGCTGTTCAAAATGTTCCCCTGGACAGCA |
| | | TGCTAAGGTGTTTTGTACCAAGACAAGTGACACTGTCTGTGACAGCTGTGAGGATTCT |
| | | ACTTACACCCAGCTGTGGAATTGGGTGCCAGAATGTCTGTCATGTGGGAGCAGGTGTT |
| | | CTAGTGACCAGGTGGAAACTCAGGCCTGTACAAGAGAGCAGAACAGGATTTGTACATG |
| | | TAGACCAGGGTGGTACTGTGCCCTGAGCAAACAGGAAGGGTGTAGGCTCTGTGCTCCC |
| | | CTGAGGAAATGTAGACCTGGGTTTGGGGTGGCCAGACCAGGAACAGAAACTTCTGATG |
| | | TGGTCTGTAAGCCCTGTGCACCAGGAACCTTTTCAAACACTACCAGCAGTACAGACAT |
| | | CTGCAGACCCCATCAGATCTGTAATGTGGTGGCCATTCCTGGGAATGCAAGTATGGAT |
| | | GCTGTCTGTACTTCCACCAGCCCAACTAGATCCATGGCCCCAGGGGCAGTCCACCTGC |
| | | CCCAGCCTGTCAGCACTAGAAGCCAGCATACTCAGCCCACACCTGAGCCTTCTACAGC |
| | | ACCCTCCACCTCTTTCCTGCTGCCTATGGGGCCATCACCTCCAGCTGAGGGCAGTACA |
| | | GGGGATGAACCTAAGTCATGTGACAAAACCCATACCTGTCCACCCTGCCCTGCTCCTG |
| | | AGCTGCTGGGGGGACCCAGTGTCTTCCTGTTTCCTCCCAAACCTAAAGATACTCTGAT |
| | | GATCAGCAGAACCCCAGAGGTCACATGCGTGGTAGTGGATGTCTCCCATGAAGACCCT |
| | | GAAGTGAAGTTTAATTGGTATGTAGATGGGGTGGAGGTCCATAATGCAAAGACCAAGC |
| | | CCAGGGAAGAACAGTATAACAGTACTTACAGGGTGGTGTCTGTGCTGACAGTACTGCA |
| | | CCAGGACTGGCTGAATGGGAAGGAGTATAAGTGCAAAGTCAGCAATAAGGCACTGCCT |
| | | GCCCCTATTGAAAAGACAATTAGTAAAGCTAAGGGGCAGCCCAGGGAACCACAGGTCT |
| | | ACACTCTGCCCCCTAGTAGAGAGGAAATGACAAAGAATCAGGTCAGTCTGACATGTCT |
| | | GGTCAAGGGATTCTATCCAAGTGATATTGCAGTGGAGTGGGAAAGTAATGGCCAGCCA |
| | | GAGAATAACTACAAGACTACCCCCCCAGTGCTGGACAGTGATGGATCTTTCTTTCTGT |
| | | ACTCCAAACTGACTGTGGATAAATCAAGATGGCAGCAGGGCAATGTCTTTTCCTGTTC |
| | | AGTCATGCATGAAGCCCTGCATAATCACTACACTCAGAAGAGCCTGTCCCTGTCCCCT |
| | | GGCAAATGATAACtcgaggacggggtgaactacgcctgaggatccgatctttttccct |
| | | ctgccaaaaattatggggacatcatgaagccccttgagcatctgacttctggctaata |
| | | aaggaaatttattttcattgcaatagtgtgttggaattttttgtgtctctcactcg |
| pAAV.sc.mU1a. | SEQ ID | ctgcgcgctcgctcgctcactgaggccgcccgggcaaagcccgggcgtcgggcgacct |
| Vh4i. | NO: 17 | ttggtcgcccggcctcagtgagcgagcgagcgcgcagagagggagtggaattcaCGCG |
| Etanercept | | TATGGAGGCGGTACTATGTAGATGAGAATTCAGGAGCAAACTGGGAAAAGCAACTGCT |
| (5'-ITR to | | TCCAAATATTTGTGATTTTTACAGTGTAGTTTTGGAAAAACTCTTAGCCTACCAATTC |
| 3'-ITR) | | TTCTAAGTGTTTTAAAATGTGGGAGCCAGTACACATGAAGTTATAGAGTGTTTTAATG |
| (2259 bp) | | AGGCTTAAATATTTACCGTAACTATGAAATGCTACGCATATCATGCTGTTCAGGCTCC |
| | | GTGGCCACGCAACTCATACTCAGGTGAGTATCTCAGGGATCCAGACATGGGGATATGG |
| | | GAGGTGCCTCTGATCCCAGGGCTCACTGTGGGTCTCTCTGTTCACAGGTTAccggtGC |
| | | CACCATGTATAGGATGCAGCTGCTGCTGCTGATTGCTCTGAGTCTGGCTCTGGTCACA |
| | | AATAGTCTGCCTGCACAGGTGGCTTTTACCCCATATGCTCCTGAGCCTGGATCCACTT |
| | | GTAGGCTGAGGGAGTATTATGATCAGACAGCCCAGATGTGCTGTTCAAAATGTTCCCC |
| | | TGGACAGCATGCTAAGGTGTTTTGTACCAAGACAAGTGACACTGTCTGTGACAGCTGT |
| | | GAGGATTCTACTTACACCCAGCTGTGGAATTGGGTGCCAGAATGTCTGTCATGTGGGA |
| | | GCAGGTGTTCTAGTGACCAGGTGGAAACTCAGGCCTGTACAAGAGAGCAGAACAGGAT |
| | | TTGTACATGTAGACCAGGGTGGTACTGTGCCCTGAGCAAACAGGAAGGGTGTAGGCTC |
| | | TGTGCTCCCCTGAGGAAATGTAGACCTGGGTTTGGGGTGGCCAGACCAGGAACAGAAA |
| | | CTTCTGATGTGGTCTGTAAGCCCTGTGCACCAGGAACCTTTTCAAACACTACCAGCAG |
| | | TACAGACATCTGCAGACCCCATCAGATCTGTAATGTGGTGGCCATTCCTGGGAATGCA |
| | | AGTATGGATGCTGTCTGTACTTCCACCAGCCCAACTAGATCCATGGCCCCAGGGGCAG |
| | | TCCACCTGCCCCAGCCTGTCAGCACTAGAAGCCAGCATACTCAGCCCACACCTGAGCC |
| | | TTCTACAGCACCCTCCACCTCTTTCCTGCTGCCTATGGGGCCATCACCTCCAGCTGAG |
| | | GGCAGTACAGGGGATGAACCTAAGTCATGTGACAAAACCCATACCTGTCCACCCTGCC |
| | | CTGCTCCTGAGCTGCTGGGGGGACCCAGTGTCTTCCTGTTTCCTCCCAAACCTAAAGA |
| | | TACTCTGATGATCAGCAGAACCCCAGAGGTCACATGCGTGGTAGTGGATGTCTCCCAT |
| | | GAAGACCCTGAAGTGAAGTTTAATTGGTATGTAGATGGGGTGGAGGTCCATAATGCAA |
| | | AGACCAAGCCCAGGGAAGAACAGTATAACAGTACTTACAGGGTGGTGTCTGTGCTGAC |
| | | AGTACTGCACCAGGACTGGCTGAATGGGAAGGAGTATAAGTGCAAAGTCAGCAATAAG |
| | | GCACTGCCTGCCCCTATTGAAAAGACAATTAGTAAAGCTAAGGGGCAGCCCAGGGAAC |
| | | CACAGGTCTACACTCTGCCCCCTAGTAGAGAGGAAATGACAAAGAATCAGGTCAGTCT |
| | | GACATGTCTGGTCAAGGGATTCTATCCAAGTGATATTGCAGTGGAGTGGGAAAGTAAT |
| | | GGCCAGCCAGAGAATAACTACAAGACTACCCCCCCAGTGCTGGACAGTGATGGATCTT |
| | | TCTTTCTGTACTCCAAACTGACTGTGGATAAATCAAGATGGCAGCAGGGCAATGTCTT |
| | | TTCCTGTTCAGTCATGCATGAAGCCCTGCATAATCACTACACTCAGAAGAGCCTGTCC |
| | | CTGTCCCCTGGCAAATGATAAaagcttgatctttttccctctgccaaaaattatgggg |
| | | acatcatgaagccccttgagcatctgacttctggctaataaaggaaatttattttcat |
| | | tgcaatagtgtgttggaattttttgtgtctctcactcggctagcgaagcaattctagc |
| | | aggcatgctggggagagatcgatctgaggaacccctagtgatggagttggccactccc |
| | | tctctgcgcgctcgctcgctcactgaggccgcccgggcaaagcccgggcgtcgggcga |
| | | cctttggtcgcccggcctcagtgagcgagcgagcgcgcagagagggagtggccaa |
| pAAV.sc.mU1a. | SEQ ID | ATGGAGGCGGTACTATGTAGATGAGAATTCAGGAGCAAACTGGGAAAAGCAACTGCTT |
| Vh4i. | NO: 18 | CCAAATATTTGTGATTTTTACAGTGTAGTTTTGGAAAAACTCTTAGCCTACCAATTCT |
| Etanercept | | TCTAAGTGTTTTAAAATGTGGGAGCCAGTACACATGAAGTTATAGAGTGTTTTAATGA |
| (promoter to | | GGCTTAAATATTTACCGTAACTATGAAATGCTACGCATATCATGCTGTTCAGGCTCCG |
| polyA) | | TGGCCACGCAACTCATACTCAGGTGAGTATCTCAGGGATCCAGACATGGGGATATGGG |
| (1951 bp) | | AGGTGCCTCTGATCCCAGGGCTCACTGTGGGTCTCTCTGTTCACAGGTTAccggtGCC |
| | | ACCATGTATAGGATGCAGCTGCTGCTGCTGATTGCTCTGAGTCTGGCTCTGGTCACAA |
| | | ATAGTCTGCCTGCACAGGTGGCTTTTACCCCATATGCTCCTGAGCCTGGATCCACTTG |
| | | TAGGCTGAGGGAGTATTATGATCAGACAGCCCAGATGTGCTGTTCAAAATGTTCCCCT |
| | | GGACAGCATGCTAAGGTGTTTTGTACCAAGACAAGTGACACTGTCTGTGACAGCTGTG |
| | | AGGATTCTACTTACACCCAGCTGTGGAATTGGGTGCCAGAATGTCTGTCATGTGGGAG |
| | | CAGGTGTTCTAGTGACCAGGTGGAAACTCAGGCCTGTACAAGAGAGCAGAACAGGATT |
| | | TGTACATGTAGACCAGGGTGGTACTGTGCCCTGAGCAAACAGGAAGGGTGTAGGCTCT |
| | | GTGCTCCCCTGAGGAAATGTAGACCTGGGTTTGGGGTGGCCAGACCAGGAACAGAAAC |
| | | TTCTGATGTGGTCTGTAAGCCCTGTGCACCAGGAACCTTTTCAAACACTACCAGCAGT |
| | | ACAGACATCTGCAGACCCCATCAGATCTGTAATGTGGTGGCCATTCCTGGGAATGCAA |
| | | GTATGGATGCTGTCTGTACTTCCACCAGCCCAACTAGATCCATGGCCCCAGGGGCAGT |
| | | CCACCTGCCCCAGCCTGTCAGCACTAGAAGCCAGCATACTCAGCCCACACCTGAGCCT |
| | | TCTACAGCACCCTCCACCTCTTTCCTGCTGCCTATGGGGCCATCACCTCCAGCTGAGG |
| | | GCAGTACAGGGGATGAACCTAAGTCATGTGACAAAACCCATACCTGTCCACCCTGCCC |
| | | TGCTCCTGAGCTGCTGGGGGGACCCAGTGTCTTCCTGTTTCCTCCCAAACCTAAAGAT |
| | | ACTCTGATGATCAGCAGAACCCCAGAGGTCACATGCGTGGTAGTGGATGTCTCCCATG |
| | | AAGACCCTGAAGTGAAGTTTAATTGGTATGTAGATGGGGTGGAGGTCCATAATGCAAA |
| | | GACCAAGCCCAGGGAAGAACAGTATAACAGTACTTACAGGGTGGTGTCTGTGCTGACA |
| | | GTACTGCACCAGGACTGGCTGAATGGGAAGGAGTATAAGTGCAAAGTCAGCAATAAGG |
| | | CACTGCCTGCCCCTATTGAAAAGACAATTAGTAAAGCTAAGGGGCAGCCCAGGGAACC |
| | | ACAGGTCTACACTCTGCCCCCTAGTAGAGAGGAAATGACAAAGAATCAGGTCAGTCTG |
| | | ACATGTCTGGTCAAGGGATTCTATCCAAGTGATATTGCAGTGGAGTGGGAAAGTAATG |
| | | GCCAGCCAGAGAATAACTACAAGACTACCCCCCCAGTGCTGGACAGTGATGGATCTTT |
| | | CTTTCTGTACTCCAAACTGACTGTGGATAAATCAAGATGGCAGCAGGGCAATGTCTTT |
| | | TCCTGTTCAGTCATGCATGAAGCCCTGCATAATCACTACACTCAGAAGAGCCTGTCCC |
| | | TGTCCCCTGGCAAATGATAAaagcttgatctttttccctctgccaaaaattatgggga |
| | | catcatgaagccccttgagcatctgacttctggctaataaaggaaatttattttcatt |
| | | gcaatagtgtgttggaattttttgtgtctctcactcg |

### 5.6 Dose Administration of Anti-TNFα Fusion Proteins

Therapeutically effective doses of any such recombinant vector should be administered in any manner such that the recombinant vector enters ocular tissue cells (e.g., retinal cells), e.g. by introducing the recombinant vector into the bloodstream. Alternatively, the vector may be administered directly to the eye, e.g., via subretinal, intravitreal, intracameral, suprachoroidal injection. In specific, embodiments, the vector is administered subretinally, intravitreally, intracamerally, suprachoroidally, subcutaneously, intramuscularly or intravenously. Subretinal, intravitreal, intracamerally, or suprachoroidal administration should result in expression of the soluble transgene product in one or more of the following retinal cell types: human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells or other ocular tissue cell: cornea cells, iris cells, ciliary body cells, a schlemm's canal cells, a trabecular meshwork cells, RPE-choroid tissue cells, or optic nerve cells.

The expression of the transgene product results in delivery and maintenance of the transgene product in one or more ocular issues or ocular tissue cell types, e.g. retinal cells. Pharmaceutical compositions suitable for administration comprise a suspension of the recombinant vector comprising the transgene encoding anti-TNFα Fc fusion protein in a formulation buffer comprising a physiologically compatible aqueous buffer. The formulation buffer can comprise one or more of a polysaccharide, a surfactant, polymer, or oil.

Alternatively, vector encoding the recombinant vector encoding the anti-TNFα Fc fusion is delivered to the liver and the anti-TNFα Fc fusion is expressed and secreted into the circulation. In specific embodiments, doses and routes of administration of a vector comprising the transgene encoding an etanercept Fc fusion protein should result in expression of the etanercept Fc fusion protein that achieve and maintain an intravitreal concentration of the etanercept fusion protein at an equivalent level to the intravitreal concentration achieved by monthly injections of etanercept (Amgen) at a dose of 40 mg.

### 5.7 Monitoring of Efficacy

The compositions and methods described herein may be assessed for efficacy using any method for assessing efficacy in treating, preventing, or ameliorating NIU. The assessment may be determined in animal models or in human subjects. The efficacy on visual deficits may be measured by best corrected visual acuity (BCVA), for example, assessing the increase in numbers of letters or lines and where efficacy may be assessed as an increase in greater than or equal to 2 ETDRS lines or an increase in log MAR, reduced inflammatory activity of the anterior and posterior chamber according to the SUN classification, and/or reduction in grade of vitreous haze. Physical changes to the eye may be measured by Optical Coherence Tomography, using methods known in the aft.

The compositions and methods described herein may be assessed for efficacy using any method for assessing efficacy in treating, preventing, or ameliorating NIU. The assessment may be determined in animal models or in human subjects. The efficacy on visual deficits may be measured by best corrected visual acuity (BCVA), for example, assessing the increase in numbers of letters or lines and where efficacy may be assessed as an increase in greater than or equal to 2 ETDRS lines or an increase in log MAR, reduced inflammatory activity of the anterior and posterior chamber according to the SUN classification, and/or reduction in grade of vitreous haze. Physical changes to the eye may be measured by Optical Coherence Tomography, using methods known in the aft. Efficacy may further be monitored by determining flare and/or relapse rates, anterior chamber cell, vitreous cell, and vitreous haze grades (e.g. grade of ≤0.5+), and/or number of active retinal or choroidal (inflammatory) lesions (e.g. see Kim J. S. et al, Int Ophthalmol Clin. 2015 Summer; 55(3): 79-110 or Rosenbaum J. T. et al Volume 49, Issue 3, December 2019, Pages 438-445; which are incorporated by reference herein in its entirety).

Endpoints may include, but are not limited to, mean change in vitreous haze grade in the study eye from baseline to 12, 16, 20, 24, or 28 weeks or at time of rescue, if earlier, proportion of responders with no recurrence of active intermediate, posterior, or panuveitis in the study eye at 12, 16, 20, 24, or 28 weeks, mean change in best corrected visual acuity from baseline to 12, 16, 20, 24, or 28 weeks, change from baseline in quality of life/patient reported outcome assessments, mean change in vitreous haze grade and anterior chamber cell grade from baseline to 12, 16, 20, 24, or 28 weeks, or change in immunosuppressive medication score from baseline to 12, 16, 20, 24, or 28 weeks.

### 6. EXAMPLES

### 6.1 Example 1: Etanercept cDNA-Based Vector

A etanercept cDNA-based vector was constructed comprising a transgene comprising nucleotide sequences encoding the TNFR:Fc fusion etanercept (amino acid sequences being SEQ ID NO: 10 or 11). Nucleotide sequences are the nucleotide sequence of SEQ ID NO: 13 or 14. The transgene also included nucleotide sequences that encodes a signal peptide, e.g., MYRMQLLLLIALSLALVTNS (SEQ ID NO:62). See FIG. 2A for amino acid sequence of the transgene product. The vector additionally includes a constitutive promoter, such as CAG and mU1a, and can include alternative promoters such as EFla or CB7 or CB (SEQ ID NO: 159) or CB long (SEQ ID NO: 160) promoter, or a tissue-specific promoter, such as a ocular tissue-specific promoter, particularly GRK1 promoter (SEQ ID NO:54), or a Best1/GRK promoter (SEQ ID NO: 161) or an inducible promoter, such as a hypoxia-inducible promoter.

### 6.2 Example 2: EYS060 cDNA-Based Vector

A EYS060 cDNA-based vector is constructed comprising a transgene comprising nucleotide sequences encoding the TNFR:Fc fusion EYS060 (amino acid sequences being SEQ ID NO: 12). The transgene also comprises nucleotide sequences that encodes a signal peptide, e.g., MYRMQLLLLIALSLALVTNS (SEQ ID NO:62). See FIG. 2B for amino acid sequence of a transgene product. The vector additionally includes a constitutive promoter, such as CAG, mU1a, and can include alternative promoters such as EFla or CB7 or CB (SEQ ID NO: 159) or CB long (SEQ ID NO: 160) promoter, or a tissue-specific promoter, such as a ocular tissue-specific promoter, particularly GRK1 promoter (SEQ ID NO:54), or a Best1/GRK promoter (SEQ ID NO: 161), or an inducible promoter, such as a hypoxia-inducible promoter.

### 6.3 Example 3: TNFα Binding Across Model Species with Vectorized Adalimumab IgG and Fab and Vectorized Etanercept

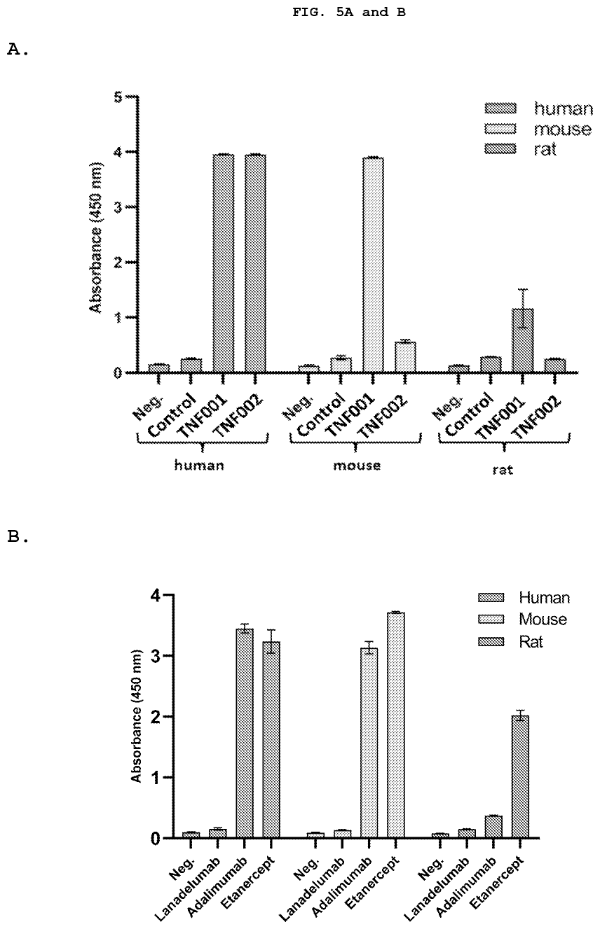

A. Vectorized adalimumab candidates isolated from model species including human, mouse, and rat were tested for their binding capacity for TNFα. Vectorized antibodies were expressed and secreted into cell supernatant following cis plasmid transfection into 293T cells. The cell supernatant was tested in an ELISA where the plates were coated with recombinant TNFα derived from the aforementioned species (FIG. 5A). Adalimumab IgG effectively bound human and mouse derived TNFα. The Fab demonstrates a similar binding profile to human TNFα as the IgG. However, the Fab displays poor binding to mouse TNFα compared to adalimumab IgG. Both IgG and Fab display far reduced binding to rat TNFα.
B. Vectorized etanercept was assayed in the same manner for binding to human, mouse and rat TNFα. Etanercept bound all of human, mouse and rat derived TNFα and binds rat TNFα better than adalimumab. See FIG. 5B.

### 6.4 Example 4: AAV-Mediated Ocular Gene Therapy for Non-Infectious Posterior Uveitis

Non-infectious posterior uveitis is a form of ocular inflammation that affects the retina and choroid of the eye and leads to blindness. It afflicts approximately 38,000 Americans per year. Patients are usually treated with systemic steroids or corticosteroids therapy, which results in high risks of systemic complications. In 2016, Humira (adalimumab), a human monoclonal antibody that targets tumor necrosis factor-alpha (TNFα), was approved by FDA and became the only systemic noncorticosteroid agent for the treatment of non-infectious uveitis (NIU) and has been widely used since then. In this study, a vectorized anti-TNFα Fc fusion protein, including CAG.etanercept or mU1a.Vh4i.etanercept.scAAV, or a vectorized EYS606, as well as AAV8.CAG.GFP, will be evaluated for AAV-mediated anti-TNFα Fc Fusion protein expression in vivo in mouse ocular tissues via local administration. AAV8.NUL will serve as a control vector. Efficacy studies in rodent EAU model will also be performed to investigate therapeutic potential of AAV-mediated anti-TNFα treatment for NIU.

Vectorized etanercept and EYS606 sequences will be constructed and tested in vitro. The transduction efficiency and cell type specificity in wild type mouse will further be evaluated. Young adult C57BL/6 and B10.RIII mice (8-10 weeks old) will be used for this study. Vectors including CAG.etanercept (SEQ ID NO: 15 or 16 (with and without flanking ITRs)) or mU1a.Vh4i.etanercept.scAAV (SEQ ID NO: 17 or 18 (with or without flanking ITR sequences)), or a vectorized EYS606, as well as, AAV8.CAG.GFP and AAV8.NUL will be delivered in mouse eyes via subretinal (SR) injection at different doses (1×10⁷, 1×10⁸ and 1×10⁹ vg/eye) in 1 μl of formulation buffer. Fundus and OCT imaging will be performed at 1, 2 and 4 weeks after SR injection. Ocular samples will be collected at 5 weeks post administration. Levels of antibody or fusion protein expression in ocular tissues will be quantified by ELISA. Cell type specificity will be determined by immunofluorescent staining with various retinal cell markers. Test vector(s) will be selected for efficacy study. Different routes of administration (ROA) including suprachoroidal, intracameral and intravitreal injections will also be explored. The preferred ROA will be used for efficacy study.

Efficacy studies will be conducted by inducing experimental autoimmune uveitis (EAU) in B10.RIII mice by immunization with the human IRBP peptide. T-cell mediated ocular autoimmune response will occur in this model with a peak from approximately 11 to 18 days post-induction. Test vector will be administrated in mouse eye via preferred ROA at 2 weeks before or 1 week after induction of EAU. Contralateral eye will be delivered with AAV.NUL vector and serve as a control. Fundus and OCT imaging, electroretinography (ERG) and Optokinetic nystagmus (OKN) will be tested at 10, 17 and 30 days following induction of EAU to monitor the progress of the disease. Ocular tissues or whole eyeballs will be collected at 5 weeks post EAU induction. Levels of or fusion protein expression in ocular tissues will be detected and quantified by ELISA. Retina structure changes and neuron survival will be evaluated by histology and immunofluorescent staining.

### 6.5 Example 5: In Vivo Study

In this study, full length and Fab adalimumab antibody in an adeno-associated virus (AAV) vector (AAV8.CAG.adalimumab.IgG (NIU001) and AAV8.CAG.adalimumab.Fab (NIU002)), as well as etanercept Fc fusion protein (AAV8.CAG.etanercept), will be evaluated for AAV-mediated antibody expression in vivo in mouse ocular tissues via local administration (subretinal (SR), Table 8).

TABLE 8
Study Layout

| | | | Inj. | | | |
|---|---|---|---|---|---|---|
| | | | Volume | Dose | N | N |
| Group | Vector | ROA | (uL) | (vg/eye) | (animals) | (eyes) |
| 1 | AAV8.CAG.Adalimumab.IgG | SR | 1 | 1.00E+09 | 2 | 4 |
| 2 | | SR | 1 | 1.00E+08 | 2 | 4 |
| 3 | AAV8.CAG.Adalimumab.Fab | SR | 1 | 1.00E+09 | 3 | 6 |
| 4 | | SR | 1 | 1.00E+08 | 3 | 6 |
| 5 | AAV8.CAG.Etanercept | SR | 1 | 1.00E+09 | 3 | 6 |
| 6 | | SR | 1 | 1.00E+08 | 3 | 6 |
| 7 | Vehicle | SR | 1 | 1.00E+09 | 2 | 4 |

Vectorized adalimumab and etanercept sequences have been constructed and tested in vitro. Young adult B10.RIII mice (6-8 weeks old) were used for this study. Vectors including AAV8.CAG.adalimumab.IgG, AAV8.CAG.adalimumab.Fab, AAV8.CAG.etanercept, and vehicle are delivered in mouse eyes via subretinal (SR) injection at two different doses (1×10⁸ and 1×10⁹ vg/eye) in 1 μl of formulation buffer (Table 8).

Fundus and OCT imaging were performed at 2 and 4 weeks after SR injection. Ocular samples were collected at 4 weeks post administration. Levels of antibody or fusion protein expression in ocular tissues are quantified by ELISA. Cell type specificity was determined by immunofluorescent staining with various retinal cell markers. Retina structure changes and neuron survival are evaluated by histology and immunofluorescent staining at 2 and 4 weeks post administration.

### 6.6 Example 6: Evaluation of Vector-Expressed Etanercept Binding Kinetics

Expression and purification of vectorized etanercept from AAV produced from cis plasmids will be assessed. The purified vectorized etanercept kinetics of binding to various species of TNFα protein will be compared to commercially produced etanercept in various ligand binding assays.

Binding affinity using Biacore™ (surface plasmon resonance (SPR)) assays: A study is performed to measure the binding affinity of different TNF-alpha (TNFα) molecules to purified TNFR-fusion proteins using BiacoreT200. First, binding affinity of TNFα to pAAV.CAG.Etanercept-produced fusion protein is compared to binding of TNFα to commercial etanercept. Second, binding affinity of TNFα from different species are tested in order to determine the suitability of various species TNFα proteins for later animal model studies. The Biacore assay is performed at 25° C. using HBS-EP+ as the running buffer. Diluted fusion proteins are captured on the sensor chip through Fc capture method (15-20 minutes capture time). Different species TNFα proteins (human, macaque, porcine, mouse, canine, rabbit and rat) can be tested individually as the analyte, followed by injecting running buffer in the dissociation phase. Dissociation rates are calculated [Koff=Kd=protein dissociation rate; Kon=Ka=protein association rate; K¬D=Koff/Kon], and smaller (lower) KD values indicated the greater the affinity of the TNFR-fusion protein for its target.

TABLE 9
Assay setup

| Ligand | Analyte |
|---|---|
| Etanercept | Human TNFα |
| pAAV.CAG.Etanercept | Human TNFα |
| Etanercept | Macaque TNFα |
| pAAV.CAG.Etanercept | Macaque TNFα |
| Etanercept | Porcine TNFα |
| pAAV.CAG.Etanercept | Porcine TNFα |
| Etanercept | Mouse TNFα |
| pAAV.CAG.Etanercept | Mouse TNFα |
| Etanercept | Canine TNFα |
| pAAV.CAG.Etanercept | Canine TNFα |
| Etanercept | Rabbit TNFα |
| pAAV.CAG.Etanercept | Rabbit TNFα |
| Etanercept | Rat TNFα |
| pAAV.CAG.Etanercept | Rat TNFα |

Binding affinity (KD) of different species TNFα to vectorized etanercept vs. commercial etanercept will be ranked according to their KD value.

### 6.7 Example 7: Measurement of TNF-Fusion Protein Effector Function

Effector functions, antibody-dependent cell-mediated cytotoxicity (ADCC) and complement-dependent cytotoxicity (CDC), of the vector-produced etanercept will be evaluated by in vitro assays and compared to commercially produced etanercept (ENBREL). Effector functions are elicited through the Fc domain of antibodies and thus, fusion proteins containing an Fc domain, such as etanercept.

### Materials and Methods

Target cells (CHO/DG44-tm TNFα; GenScript Cat. #RD00746) are maintained with corresponding complete culture medium at 37° C. with 5% CO2. Effector cells (peripheral blood mononuclear cells, PBMCs; Saily Bio Cat. #XFB-HP100B) are thawed at 37° C. and maintained with 1640 complete culture medium at 37° C. with 5% CO2.

For the ADCC dose-response assay, CHO/DG44-tm TNFα and PBMCs can be used as target and effector cells, respectively. With a E/T (effector cell to target cell) ratio set at 25:1, etanercept (commercial) and human IgG1 against CHO/DG44-tm TNFα re used as positive and negative control, respectively. Briefly, the method steps are:

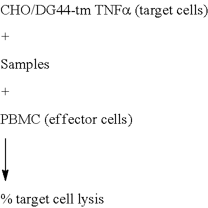

Effector cells (PBMCs) are thawed and resuspended with assay buffer (CellTiter-Glo® Detection Kit (Promega, Cat. #G7573). Target cells are also thawed and resuspended with ADCC assay buffer, then transferred in suspension to an assay plate following a plate map. Controls and test samples in solution are transferred to the assay plate as well, and the assay plate incubated at RT for 30 minutes. The effector cell density is adjusted according to the E/T ratio, then the effector cell suspension is transferred to the assay plate. The assay plate is then incubated in a cell incubator (37° C./5% CO2) for 6 hours, removed, then the supernatant of corresponding wells of the assay plate are transferred to another 96-well assay plate. LDH Mixture (LDH Cytotoxicity Detection Kit, Roche Cat #11644793001) is transferred to the corresponding wells of the second 96-well assay plate and luminescence/absorbance is read with a PHERAStar® (BMG LABTECH) plate reader.

For the CDC dose-response study, CHO/DG44-tm TNFα can be used as the target cell. With 5% NHSC (normal human serum complement), etanercept and human IgG1 against CHO/DG44-tm TNFα can be used as positive and negative control, respectively. Briefly, the CDC assay method steps are:

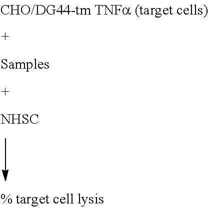

Target cells are harvested by centrifugation and resuspended with assay buffer (CellTiter-Glo® Detection Kit (Promega, Cat. #G7573). Samples and controls are prepared in solution with CDC assay buffer. Target cell density was adjusted and then cell suspension transferred to the assay plate. Controls and test samples in working solution are also transferred to the assay plate, and then assay plate is incubated at RT for 30 minutes, before the Normal Human Serum Complement (NHSC) working solution (Quidel, Cat. #A113) is added to the assay plate. The assay plate is incubated in the cell incubator (37° C./5% CO2) for 4 hours, removed, and the Cell Titer-Glo® working solution is added to the corresponding wells and the plate incubated for about 10-30 minutes at RT. Luminescence data is read on a PHERAStar® FSX (BMG LABTECH) plate reader to determine the number of viable cells. Raw data of ADCC and CDC study are exported from the PHERAStar® FSX system and analyzed using Microsoft Office Excel 2016 and GraphPad Prism 6 software. The formula of ADCC % Target cell lysis=100*(ODSamples−ODTumor cells plus effector cells)/(ODMaximum release−ODMinimum release). The formula of CDC % Target cell lysis=100*(1−(RLUSamples−RLUNHSC)/(RLUCell+NHSC−RLUNHSC)). Relative EC50 values are obtained using four-parameter function as follows, characterizing sigmoid curve where % target cell lysis is against the concentration of the test samples: Y=Bottom+(Top−Bottom)/(1+10\hat{ }((Log EC50−X)*HillSlope))=Percentage of target cell lysis; and X=Concentration.

With an E/T ratio at 25:1, CHO/DG44-tm TNFα cells can be used as the target cells in a ADCC dose-response study. Dose-responses and Best-fit values of positive control (commercial etanercept), samples and negative control (Human IgG1) are calculated (e.g. EC50).

With 5% NHSC, CHO/DG44-tm TNFα cells are used as the target cells in CDC dose-response study. Dose-responses and Best-fit values of positive control (etanercept), samples and negative control (Human IgG1) are calculated (e.g. EC50).

AAV-etanercept ADCC and CDC activity will be compared to commercial etanercept. Without being bound to any one theory, differences may be observed due to the post-translational modification, such as glycosylation, which is expected to differ due to manufacturing cell culture of commercial etanercept.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

1-19. (canceled)

20. A composition comprising an adeno-associated virus (AAV) vector comprising:
(a) a viral AAV capsid,
(b) an artificial genome comprising an expression cassette flanked by AAV inverted terminal repeats (ITRs), wherein the expression cassette comprises a transgene encoding an anti-TNF-α Fc fusion protein comprising a human soluble TNFα receptor type I (TNFR1) or type II (TNFR2) extracellular domain covalently linked through a peptide bond to a polypeptide comprising an Fc domain of an immunoglobulin heavy chain, operably linked to one or more regulatory sequences that promote expression of the transgene in human ocular tissue cells;
wherein the transgene encodes a signal sequence at the N-terminus of said anti-TNFα Fc fusion protein, wherein said signal sequence directs secretion and post translational modification of said anti-TNFα Fc fusion in ocular tissue cells.

21. The composition of claim 20, wherein the transgene further comprises a thrombin cleavage site of SEQ ID NO:8 at the C-terminus of soluble human TNFR extracellular domain.

22. The composition of any of claim 20, wherein the anti-TNFα Fc fusion protein has an amino acid sequence of SEQ ID NO: 10, 11, or 12.

23. The composition of claim 20, wherein the anti-TNFα Fc fusion protein is etanercept or EYS606.

24. The composition of claim 20, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.

25. The composition of claim 20, wherein said transgene comprises the nucleotide sequence of SEQ ID NO:13 or 14.

26. The composition of claim 20, wherein said signal sequence is MYRMQLLLLIALSLALVTNS (SEQ ID NO:62) or a signal sequence from Tables 2 or 3.

27. The composition of any of claim 20, wherein the artificial genome is self complementary.

28. The composition of claim 20, wherein the artificial genome is the construct CAG.etanercept (SEQ ID NO: 15 or 16) or mU1a.Vh4i.etanercept.scAAV (SEQ ID NO: 17 or 18).

29. A method of treating non-infectious uveitis in a human subject in need thereof, comprising subretinally, intravitreally, intranasally, intracamerally, or suprachoroidally administering to the subject a therapeutically effective amount of a composition comprising (a) a viral capsid, and (b) an artificial genome comprising an expression cassette flanked by AAV internal tandem repeats (ITRs), wherein the expression cassette comprises a transgene encoding an anti-TNFα Fc fusion protein operably linked to one or more regulatory sequences that control expression of the transgene in ocular tissue cells.

30. (canceled)

31. The method of claim 29, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.

32. The method of claim 29, wherein said anti-TNF-α Fc fusion protein comprises a human soluble TNFα receptor type I (TNFR1) or type II (TNFR2) extracellular domain covalently linked to an IgG Fc domain.

33. The method of claim 29, wherein said transgene is etanercept or EYS606.

34. The method of claim 29, wherein said transgene has the nucleotide sequence of SEQ ID NO: 13-18.

35. The method of claim 29, wherein the viral capsid is at least 95% identical to the amino acid sequence of AAV serotype 1 (AAV1), serotype 2 (AAV2), serotype AAV2.7m8, serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

36. (canceled)

37. (canceled)

38. The method of claim 29, wherein the regulatory sequence is a human rhodopsin kinase (GRK1) promoter (SEQ ID NOS:54 or 117), a mouse cone arresting (CAR) promoter (SEQ ID NOS:114-116), or a human red opsin (RedO) promoter (SEQ ID NO:112).

39. The method of claim 29, wherein the transgene encodes a signal sequence at the N-terminus of the anti-TNFα Fc fusion protein that directs secretion and post translational modification in said human ocular tissue cells.

40. The method of claim 39, wherein said signal sequence is MYRMQLLLLIALSLALVTNS (SEQ ID NO:62).

41. The method of claim 29, wherein transgene has the structure: Signal sequence—human soluble TNFR extracellular domain (type 1 or type 2)—hinge region—Fc domain—PolyA.

42. The method of claim 41, wherein the transgene further comprises a thrombin cleavage site having SEQ ID NO:8 at the C-terminus of the human soluble TNFR extracellular domain.

43-47. (canceled)

48. The method of claim 29, wherein the therapeutically effective amount is determined to be sufficient to improve best corrected visual acuity (BCVA) by >=2 ETDRS lines or increase in log MAR, reduced inflammatory activity of the anterior and posterior chamber according to the SUN classification, and/or reduction in grade of vitreous haze.

49. The method of claim 29, wherein the rAAV is self complementary.

50. The method of claim 29 wherein the artificial genome is the construct CAG.etanercept (SEQ ID NO: 15 or 16) or mU1a.Vh4i.etanercept.scAAV (SEQ ID NO: 17 or 18).

51. A method of producing recombinant AAVs comprising:
(a) culturing a host cell comprising:
(i) an artificial genome according to claim 20;
(ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue tropism;
(iii) sufficient adenovirus helper functions to permit replication and packaging of the artificial genome by the AAV capsid protein; and
(b) recovering recombinant AAV encapsidating the artificial genome from the cell culture.

52. (canceled)

53. (canceled)

54. A host cell containing:
a plasmid comprising a cis expression cassette flanked by AAV ITRs, wherein the cis expression cassette comprising a transgene encoding an anti-TNFα fusion protein, wherein the anti-TNFα Fc fusion protein comprises a soluble, extracellular portion of human TNFα receptor type I (TNFR1) or type II (TNFR2) covalently linked through a peptide bond to polypeptide comprising an Fc domain of an immunoglobulin heavy chain, operably linked to one or more regulatory sequences that promote expression of the transgene in human ocular tissue cells.

55. (canceled)

56. (canceled)


SEQUENCE LISTING (161 sequences)

SEQ ID NO: 1 - protein, 211 aa, Artificial Sequence
  synthetic construct; Anti-TNFalpha Fc Fusion
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 2 - protein, 235 aa, Artificial Sequence
  synthetic construct; Anti-TNFalpha Fc Fusion
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 3 - protein, 232 aa, Artificial Sequence
  synthetic construct; Anti-TNFalpha Fc Fusion
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 207 aa, Artificial Sequence
  synthetic construct; Anti-TNFalpha Fc Fusion
FLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 25 aa, Artificial Sequence
  synthetic construct; hing region
EPKSCDKTHTCPPCPAPELLGGPSV

SEQ ID NO: 6 - protein, 183 aa, Artificial Sequence
  synthetic construct; EYS606 Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGS

SEQ ID NO: 7 - protein, 163 aa, Artificial Sequence
  synthetic construct; EYS606 Fc
FLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGS

SEQ ID NO: 8 - protein, 6 aa, Artificial Sequence
  synthetic construct; cleavage site
LVPRGS

SEQ ID NO: 9 - protein, 20 aa, Artificial Sequence
  synthetic construct; hinge region
DKTHTCPPCPAPELLGGPSV

SEQ ID NO: 10 - protein, 467 aa, Artificial Sequence
  synthetic construct; Etanercept TNFR2Fc fusion
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 487 aa, Artificial Sequence
  synthetic construct; Etanercept plus leader sequence
MYRMQLLLLIALSLALVTNSLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 400 aa, Artificial Sequence
  synthetic construct; EYS606
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTLVPRGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGS

SEQ ID NO: 13 - DNA, 1404 bp, Artificial Sequence
  synthetic construct; Etanercept
ctgcctgcacaggtggcttttaccccatatgctcctgagcctggatccacttgtaggctgagggagtattatgatcagacagcccagatgtgctgttcaaaatgttcccctggacagcatgctaaggtgttttgtaccaagacaagtgacactgtctgtgacagctgtgaggattctacttacacccagctgtggaattgggtgccagaatgtctgtcatgtgggagcaggtgttctagtgaccaggtggaaactcaggcctgtacaagagagcagaacaggatttgtacatgtagaccagggtggtactgtgccctgagcaaacaggaagggtgtaggctctgtgctcccctgaggaaatgtagacctgggtttggggtggccagaccaggaacagaaacttctgatgtggtctgtaagccctgtgcaccaggaaccttttcaaacactaccagcagtacagacatctgcagaccccatcagatctgtaatgtggtggccattcctgggaatgcaagtatggatgctgtctgtacttccaccagcccaactagatccatggccccaggggcagtccacctgccccagcctgtcagcactagaagccagcatactcagcccacacctgagccttctacagcaccctccacctctttcctgctgcctatggggccatcacctccagctgagggcagtacaggggatgaacctaagtcatgtgacaaaacccatacctgtccaccctgccctgctcctgagctgctggggggacccagtgtcttcctgtttcctcccaaacctaaagatactctgatgatcagcagaaccccagaggtcacatgcgtggtagtggatgtctcccatgaagaccctgaagtgaagtttaattggtatgtagatggggtggaggtccataatgcaaagaccaagcccagggaagaacagtataacagtacttacagggtggtgtctgtgctgacagtactgcaccaggactggctgaatgggaaggagtataagtgcaaagtcagcaataaggcactgcctgcccctattgaaaagacaattagtaaagctaaggggcagcccagggaaccacaggtctacactctgccccctagtagagaggaaatgacaaagaatcaggtcagtctgacatgtctggtcaagggattctatccaagtgatattgcagtggagtgggaaagtaatggccagccagagaataactacaagactacccccccagtgctggacagtgatggatctttctttctgtactccaaactgactgtggataaatcaagatggcagcagggcaatgtcttttcctgttcagtcatgcatgaagccctgcataatcactacactcagaagagcctgtccctgtcccctggcaaatga

SEQ ID NO: 14 - DNA, 1464 bp, Artificial Sequence
  synthetic construct; Etanercept plus leader sequence
atgtataggatgcagctgctgctgctgattgctctgagtctggctctggtcacaaatagtctgcctgcacaggtggcttttaccccatatgctcctgagcctggatccacttgtaggctgagggagtattatgatcagacagcccagatgtgctgttcaaaatgttcccctggacagcatgctaaggtgttttgtaccaagacaagtgacactgtctgtgacagctgtgaggattctacttacacccagctgtggaattgggtgccagaatgtctgtcatgtgggagcaggtgttctagtgaccaggtggaaactcaggcctgtacaagagagcagaacaggatttgtacatgtagaccagggtggtactgtgccctgagcaaacaggaagggtgtaggctctgtgctcccctgaggaaatgtagacctgggtttggggtggccagaccaggaacagaaacttctgatgtggtctgtaagccctgtgcaccaggaaccttttcaaacactaccagcagtacagacatctgcagaccccatcagatctgtaatgtggtggccattcctgggaatgcaagtatggatgctgtctgtacttccaccagcccaactagatccatggccccaggggcagtccacctgccccagcctgtcagcactagaagccagcatactcagcccacacctgagccttctacagcaccctccacctctttcctgctgcctatggggccatcacctccagctgagggcagtacaggggatgaacctaagtcatgtgacaaaacccatacctgtccaccctgccctgctcctgagctgctggggggacccagtgtcttcctgtttcctcccaaacctaaagatactctgatgatcagcagaaccccagaggtcacatgcgtggtagtggatgtctcccatgaagaccctgaagtgaagtttaattggtatgtagatggggtggaggtccataatgcaaagaccaagcccagggaagaacagtataacagtacttacagggtggtgtctgtgctgacagtactgcaccaggactggctgaatgggaaggagtataagtgcaaagtcagcaataaggcactgcctgcccctattgaaaagacaattagtaaagctaaggggcagcccagggaaccacaggtctacactctgccccctagtagagaggaaatgacaaagaatcaggtcagtctgacatgtctggtcaagggattctatccaagtgatattgcagtggagtgggaaagtaatggccagccagagaataactacaagactacccccccagtgctggacagtgatggatctttctttctgtactccaaactgactgtggataaatcaagatggcagcagggcaatgtcttttcctgttcagtcatgcatgaagccctgcataatcactacactcagaagagcctgtccctgtcccctggcaaatga

SEQ ID NO: 15 - DNA, 3784 bp, Artificial Sequence
  synthetic construct; pAAV.CAG.Etanercept (from 5′-ITR to 3-ITR)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtataggatgcagctgctgctgctgattgctctgagtctggctctggtcacaaatagtctgcctgcacaggtggcttttaccccatatgctcctgagcctggatccacttgtaggctgagggagtattatgatcagacagcccagatgtgctgttcaaaatgttcccctggacagcatgctaaggtgttttgtaccaagacaagtgacactgtctgtgacagctgtgaggattctacttacacccagctgtggaattgggtgccagaatgtctgtcatgtgggagcaggtgttctagtgaccaggtggaaactcaggcctgtacaagagagcagaacaggatttgtacatgtagaccagggtggtactgtgccctgagcaaacaggaagggtgtaggctctgtgctcccctgaggaaatgtagacctgggtttggggtggccagaccaggaacagaaacttctgatgtggtctgtaagccctgtgcaccaggaaccttttcaaacactaccagcagtacagacatctgcagaccccatcagatctgtaatgtggtggccattcctgggaatgcaagtatggatgctgtctgtacttccaccagcccaactagatccatggccccaggggcagtccacctgccccagcctgtcagcactagaagccagcatactcagcccacacctgagccttctacagcaccctccacctctttcctgctgcctatggggccatcacctccagctgagggcagtacaggggatgaacctaagtcatgtgacaaaacccatacctgtccaccctgccctgctcctgagctgctggggggacccagtgtcttcctgtttcctcccaaacctaaagatactctgatgatcagcagaaccccagaggtcacatgcgtggtagtggatgtctcccatgaagaccctgaagtgaagtttaattggtatgtagatggggtggaggtccataatgcaaagaccaagcccagggaagaacagtataacagtacttacagggtggtgtctgtgctgacagtactgcaccaggactggctgaatgggaaggagtataagtgcaaagtcagcaataaggcactgcctgcccctattgaaaagacaattagtaaagctaaggggcagcccagggaaccacaggtctacactctgccccctagtagagaggaaatgacaaagaatcaggtcagtctgacatgtctggtcaagggattctatccaagtgatattgcagtggagtgggaaagtaatggccagccagagaataactacaagactacccccccagtgctggacagtgatggatctttctttctgtactccaaactgactgtggataaatcaagatggcagcagggcaatgtcttttcctgttcagtcatgcatgaagccctgcataatcactacactcagaagagcctgtccctgtcccctggcaaatgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 3362 bp, Artificial Sequence
  synthetic construct; pAAV.CAG.Etanercept
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtataggatgcagctgctgctgctgattgctctgagtctggctctggtcacaaatagtctgcctgcacaggtggcttttaccccatatgctcctgagcctggatccacttgtaggctgagggagtattatgatcagacagcccagatgtgctgttcaaaatgttcccctggacagcatgctaaggtgttttgtaccaagacaagtgacactgtctgtgacagctgtgaggattctacttacacccagctgtggaattgggtgccagaatgtctgtcatgtgggagcaggtgttctagtgaccaggtggaaactcaggcctgtacaagagagcagaacaggatttgtacatgtagaccagggtggtactgtgccctgagcaaacaggaagggtgtaggctctgtgctcccctgaggaaatgtagacctgggtttggggtggccagaccaggaacagaaacttctgatgtggtctgtaagccctgtgcaccaggaaccttttcaaacactaccagcagtacagacatctgcagaccccatcagatctgtaatgtggtggccattcctgggaatgcaagtatggatgctgtctgtacttccaccagcccaactagatccatggccccaggggcagtccacctgccccagcctgtcagcactagaagccagcatactcagcccacacctgagccttctacagcaccctccacctctttcctgctgcctatggggccatcacctccagctgagggcagtacaggggatgaacctaagtcatgtgacaaaacccatacctgtccaccctgccctgctcctgagctgctggggggacccagtgtcttcctgtttcctcccaaacctaaagatactctgatgatcagcagaaccccagaggtcacatgcgtggtagtggatgtctcccatgaagaccctgaagtgaagtttaattggtatgtagatggggtggaggtccataatgcaaagaccaagcccagggaagaacagtataacagtacttacagggtggtgtctgtgctgacagtactgcaccaggactggctgaatgggaaggagtataagtgcaaagtcagcaataaggcactgcctgcccctattgaaaagacaattagtaaagctaaggggcagcccagggaaccacaggtctacactctgccccctagtagagaggaaatgacaaagaatcaggtcagtctgacatgtctggtcaagggattctatccaagtgatattgcagtggagtgggaaagtaatggccagccagagaataactacaagactacccccccagtgctggacagtgatggatctttctttctgtactccaaactgactgtggataaatcaagatggcagcagggcaatgtcttttcctgttcagtcatgcatgaagccctgcataatcactacactcagaagagcctgtccctgtcccctggcaaatgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 17 - DNA, 2259 bp, Artificial Sequence
  synthetic construct; pAAV.sc.mU1a.Vh4i.Etanercept (5′-ITR to 3′-ITR)
ctgcgcgctcgctcgctcactgaggccgcccgggcaaagcccgggcgtcgggcgacctttggtcgcccggcctcagtgagcgagcgagcgcgcagagagggagtggaattcacgcgtatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactcaggtgagtatctcagggatccagacatggggatatgggaggtgcctctgatcccagggctcactgtgggtctctctgttcacaggttaccggtgccaccatgtataggatgcagctgctgctgctgattgctctgagtctggctctggtcacaaatagtctgcctgcacaggtggcttttaccccatatgctcctgagcctggatccacttgtaggctgagggagtattatgatcagacagcccagatgtgctgttcaaaatgttcccctggacagcatgctaaggtgttttgtaccaagacaagtgacactgtctgtgacagctgtgaggattctacttacacccagctgtggaattgggtgccagaatgtctgtcatgtgggagcaggtgttctagtgaccaggtggaaactcaggcctgtacaagagagcagaacaggatttgtacatgtagaccagggtggtactgtgccctgagcaaacaggaagggtgtaggctctgtgctcccctgaggaaatgtagacctgggtttggggtggccagaccaggaacagaaacttctgatgtggtctgtaagccctgtgcaccaggaaccttttcaaacactaccagcagtacagacatctgcagaccccatcagatctgtaatgtggtggccattcctgggaatgcaagtatggatgctgtctgtacttccaccagcccaactagatccatggccccaggggcagtccacctgccccagcctgtcagcactagaagccagcatactcagcccacacctgagccttctacagcaccctccacctctttcctgctgcctatggggccatcacctccagctgagggcagtacaggggatgaacctaagtcatgtgacaaaacccatacctgtccaccctgccctgctcctgagctgctggggggacccagtgtcttcctgtttcctcccaaacctaaagatactctgatgatcagcagaaccccagaggtcacatgcgtggtagtggatgtctcccatgaagaccctgaagtgaagtttaattggtatgtagatggggtggaggtccataatgcaaagaccaagcccagggaagaacagtataacagtacttacagggtggtgtctgtgctgacagtactgcaccaggactggctgaatgggaaggagtataagtgcaaagtcagcaataaggcactgcctgcccctattgaaaagacaattagtaaagctaaggggcagcccagggaaccacaggtctacactctgccccctagtagagaggaaatgacaaagaatcaggtcagtctgacatgtctggtcaagggattctatccaagtgatattgcagtggagtgggaaagtaatggccagccagagaataactacaagactacccccccagtgctggacagtgatggatctttctttctgtactccaaactgactgtggataaatcaagatggcagcagggcaatgtcttttcctgttcagtcatgcatgaagccctgcataatcactacactcagaagagcctgtccctgtcccctggcaaatgataaaagcttgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggagagatcgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 18 - DNA, 1951 bp, Artificial Sequence
  synthetic construct; pAAV.sc.mU1a.Vh4i.Etanercept (promoter to polyA)
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactcaggtgagtatctcagggatccagacatggggatatgggaggtgcctctgatcccagggctcactgtgggtctctctgttcacaggttaccggtgccaccatgtataggatgcagctgctgctgctgattgctctgagtctggctctggtcacaaatagtctgcctgcacaggtggcttttaccccatatgctcctgagcctggatccacttgtaggctgagggagtattatgatcagacagcccagatgtgctgttcaaaatgttcccctggacagcatgctaaggtgttttgtaccaagacaagtgacactgtctgtgacagctgtgaggattctacttacacccagctgtggaattgggtgccagaatgtctgtcatgtgggagcaggtgttctagtgaccaggtggaaactcaggcctgtacaagagagcagaacaggatttgtacatgtagaccagggtggtactgtgccctgagcaaacaggaagggtgtaggctctgtgctcccctgaggaaatgtagacctgggtttggggtggccagaccaggaacagaaacttctgatgtggtctgtaagccctgtgcaccaggaaccttttcaaacactaccagcagtacagacatctgcagaccccatcagatctgtaatgtggtggccattcctgggaatgcaagtatggatgctgtctgtacttccaccagcccaactagatccatggccccaggggcagtccacctgccccagcctgtcagcactagaagccagcatactcagcccacacctgagccttctacagcaccctccacctctttcctgctgcctatggggccatcacctccagctgagggcagtacaggggatgaacctaagtcatgtgacaaaacccatacctgtccaccctgccctgctcctgagctgctggggggacccagtgtcttcctgtttcctcccaaacctaaagatactctgatgatcagcagaaccccagaggtcacatgcgtggtagtggatgtctcccatgaagaccctgaagtgaagtttaattggtatgtagatggggtggaggtccataatgcaaagaccaagcccagggaagaacagtataacagtacttacagggtggtgtctgtgctgacagtactgcaccaggactggctgaatgggaaggagtataagtgcaaagtcagcaataaggcactgcctgcccctattgaaaagacaattagtaaagctaaggggcagcccagggaaccacaggtctacactctgccccctagtagagaggaaatgacaaagaatcaggtcagtctgacatgtctggtcaagggattctatccaagtgatattgcagtggagtgggaaagtaatggccagccagagaataactacaagactacccccccagtgctggacagtgatggatctttctttctgtactccaaactgactgtggataaatcaagatggcagcagggcaatgtcttttcctgttcagtcatgcatgaagccctgcataatcactacactcagaagagcctgtccctgtcccctggcaaatgataaaagcttgatctttttccctctgccaaaaattatggggacatcatgaagccccttgagcatctgacttctggctaataaaggaaatttattttcattgcaatagtgtgttggaattttttgtgtctctcactcg

SEQ ID NO: 19 - protein, 467 aa, Artificial Sequence; identical to SEQ ID NO: 10
  synthetic construct; antibody construct

SEQ ID NO: 20 - protein, 400 aa, Artificial Sequence; identical to SEQ ID NO: 12
  synthetic construct; antibody construct

SEQ ID NO: 21 - protein, 17 aa, Artificial Sequence
  synthetic construct; hinge
DKTHTCPPCPAPELLGG

SEQ ID NO: 22 - protein, 736 aa, Artificial Sequence
  synthetic construct; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 23 - protein, 735 aa, Artificial Sequence [sequence omitted]

SEQ ID NO: 24 - protein, 736 aa, Artificial Sequence
  synthetic construct; AAV vector
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 25 - protein, 736 aa, Artificial Sequence; identical to SEQ ID NO: 24
  synthetic construct; AAV vector

SEQ ID NO: 26 - protein, 736 aa, Artificial Sequence
  synthetic construct; AAV vector
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 27 - protein, 734 aa, Artificial Sequence
  synthetic construct; AAV vector
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 28 - protein, 734 aa, Artificial Sequence; identical to SEQ ID NO: 27
  synthetic construct; AAV vector

SEQ ID NO: 29 - protein, 724 aa, Artificial Sequence
  synthetic construct; AAV vector
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 30 - protein, 736 aa, Artificial Sequence
  synthetic construct; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 31 - protein, 737 aa, Artificial Sequence
  synthetic construct; AAV vector
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 32 - protein, 738 aa, Artificial Sequence
  synthetic construct; AAV vector
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 33 - protein, 736 aa, Artificial Sequence
  synthetic construct; AAV vector
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 736 aa, Artificial Sequence
  synthetic construct; AAV vector
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 738 aa, Artificial Sequence
  synthetic construct; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 36 - protein, 738 aa, Artificial Sequence
  synthetic construct; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 37 - protein, 738 aa, Artificial Sequence
  synthetic construct; AAV vector
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 38 - protein, 738 aa, Artificial Sequence
  synthetic construct; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 39 - protein, 738 aa, Artificial Sequence
  synthetic construct; AAV vector
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 40 - protein, 738 aa, Artificial Sequence
  synthetic construct; AAV vector
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 41 - protein, 735 aa, Artificial Sequence
  synthetic construct; AAV vector
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRPSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 42 - protein, 735 aa, Artificial Sequence
  synthetic construct; AAV vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPRPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 43 - protein, 735 aa, Artificial Sequence
  synthetic construct; AAV vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 44 - protein, 738 aa, Artificial Sequence
  synthetic construct; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 45 - protein, 736 aa, Artificial Sequence
  synthetic construct; AAV vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVGEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSGYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAMASHKDNEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 46 - protein, 734 aa, Artificial Sequence
  synthetic construct; AAV vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLRQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYYLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 47 - protein, 232 aa, Artificial Sequence
  synthetic construct; IgG1
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 228 aa, Artificial Sequence
  synthetic construct; IgG2
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 49 - protein, 229 aa, Artificial Sequence
  synthetic construct; Igg4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - DNA, 1725 bp, Artificial Sequence
  synthetic construct; promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 51 - DNA, 1676 bp, Artificial Sequence
  synthetic construct; promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 52 - DNA, 251 bp, Artificial Sequence
  synthetic construct; promoter
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 53 - DNA, 212 bp, Artificial Sequence
  synthetic construct; promoter
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 54 - DNA, 295 bp, Artificial Sequence
  synthetic construct; promoter
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggc

SEQ ID NO: 55 - DNA, 127 bp, Artificial Sequence
  synthetic construct; regulatory element
gatctttttccctctgccaaaaattatggggacatcatgaagccccttgagcatctgacttctggctaataaaggaaatttattttcattgcaatagtgtgttggaattttttgtgtctctcactcg

SEQ ID NO: 56 - DNA, 133 bp, Artificial Sequence
  synthetic construct; regulatory element
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 57 - DNA, 82 bp, Artificial Sequence
  synthetic construct; regulatory element
gtgagtatctcagggatccagacatggggatatgggaggtgcctctgatcccagggctcactgtgggtctctctgttcacag

SEQ ID NO: 58 - DNA, 130 bp, Artificial Sequence
  synthetic construct; regulatory element
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 59 - DNA, 106 bp, Artificial Sequence
  synthetic construct; regulatory element
ctgcgcgctcgctcgctcactgaggccgcccgggcaaagcccgggcgtcgggcgacctttggtcgcccggcctcagtgagcgagcgagcgcgcagagagggagtgg

SEQ ID NO: 60 - DNA, 143 bp, Artificial Sequence
  synthetic construct; regulatory element
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 61 - DNA, 110 bp, Artificial Sequence
  synthetic construct; regulatory element
ttggccactccctctctgcgcgctcgctcgctcactgaggccgggcgaccaaaggtcgcccgacgcccgggctttgcccgggcggcctcagtgagcgagcgagcgcgcag

SEQ ID NO: 62 - protein, 20 aa, Artificial Sequence
  synthetic construct; signal sequence
MYRMQLLLLIALSLALVTNS

SEQ ID NO: 63 - DNA, 60 bp, Artificial Sequence
  synthetic construct; signal sequence
atgtataggatgcaactgctcctcctgattgctctgagcctggctcttgtgaccaactct

SEQ ID NO: 64 - protein, 26 aa, Artificial Sequence
  synthetic construct; signal sequence
MNFLLSWVHWSLALLLYLHHAKWSQA

SEQ ID NO: 65 - protein, 29 aa, Artificial Sequence
  synthetic construct; signal sequence
MERAAPSRRVPLPLLLLGGLALLAAGVDA

SEQ ID NO: 66 - protein, 19 aa, Artificial Sequence
  synthetic construct; signal sequence
MAPLRPLLILALLAWVALA

SEQ ID NO: 67 - protein, 18 aa, Artificial Sequence
  synthetic construct; signal sequence
MRLLAKIICLMLWAICVA

SEQ ID NO: 68 - protein, 19 aa, Artificial Sequence
  synthetic construct; signal sequence
MRLLAFLSLLALVLQETGT

SEQ ID NO: 69 - protein, 18 aa, Artificial Sequence
  synthetic construct; signal sequence
MKWVTFISLLFLFSSAYS

SEQ ID NO: 70 - protein, 18 aa, Artificial Sequence
  synthetic construct; signal sequence
MAFLWLLSCWALLGTTFG

SEQ ID NO: 71 - protein, 20 aa, Artificial Sequence
  synthetic construct; signal sequence
MYRMQLLSCIALILALVTNS

SEQ ID NO: 72 - protein, 15 aa, Artificial Sequence
  synthetic construct; signal sequence
MNLLLILTFVAAAVA

SEQ ID NO: 73 - protein, 18 aa, Artificial Sequence
  synthetic construct; signal sequence
MKWVTFISLLFLFSSAYS

SEQ ID NO: 74 - protein, 24 aa, Artificial Sequence
  synthetic construct; signal sequence
MPSSVSWGILLLAGLCCLVPVSLA

SEQ ID NO: 75 - protein, 18 aa, Artificial Sequence
  synthetic construct; signal sequence
MKAAVLTLAVLFLTGSQA

SEQ ID NO: 76 - protein, 18 aa, Artificial Sequence
  synthetic construct; signal sequence
MKLLAATVLLLTICSLEG

SEQ ID NO: 77 - protein, 27 aa, Artificial Sequence
  synthetic construct; signal sequence
MDPPRPALLALLALPALLLLLLAGARA

SEQ ID NO: 78 - protein, 28 aa, Artificial Sequence
  synthetic construct; signal sequence
MQRVNMIMAESPGLITICLLGYLLSAEC

SEQ ID NO: 79 - protein, 20 aa, Artificial Sequence
  synthetic construct; signal sequence
MGPLMVLFCLLFLYPGLADS

SEQ ID NO: 80 - protein, 18 aa, Artificial Sequence
  synthetic construct; signal sequence
MWLLVSVILISRISSVGG

SEQ ID NO: 81 - protein, 18 aa, Artificial Sequence
  synthetic construct; signal sequence
MLLLFSVILISWVSTVGG

SEQ ID NO: 82 - protein, 19 aa, Artificial Sequence
  synthetic construct; signal sequence
MFSMRIVCLVLSVVGTAWT

SEQ ID NO: 83 - protein, 30 aa, Artificial Sequence
  synthetic construct; signal sequence
MKRMVSWSFHKLKTMKHLLLLLLCVFLVKS

SEQ ID NO: 84 - protein, 26 aa, Artificial Sequence
  synthetic construct; signal sequence
MSWSLHPRNLILYFYALLFLSSTCVA

SEQ ID NO: 85 - protein, 18 aa, Artificial Sequence
  synthetic construct; signal sequence
MKSLVLLLCLAQLWGCHS

SEQ ID NO: 86 - protein, 23 aa, Artificial Sequence
  synthetic construct; signal sequence
MARVLGAPVALGLWSLCWSLAIA

SEQ ID NO: 87 - protein, 18 aa, Artificial Sequence
  synthetic construct; signal sequence
MKLITILFLCSRLLLSLT

SEQ ID NO: 88 - protein, 20 aa, Artificial Sequence
  synthetic construct; signal sequence
MSLFPSLPLLLLSMVAASYS

SEQ ID NO: 89 - protein, 19 aa, Artificial Sequence
  synthetic construct; signal sequence
MEHKEVVLLLLLFLKSGQG

SEQ ID NO: 90 - protein, 24 aa, Artificial Sequence
  synthetic construct; signal sequence
MAHVRGLQLPGCLALAALCSLVHS

SEQ ID NO: 91 - protein, 29 aa, Artificial Sequence
  synthetic construct; signal sequence
MISRMEKMTMMMKILIMFALGMNYWSCSG

SEQ ID NO: 92 - protein, 32 aa, Artificial Sequence
  synthetic construct; signal sequence
MYSNVIGTVTSGKRKVYLLSLLLIGFWDCVTC

SEQ ID NO: 93 - protein, 19 aa, Artificial Sequence
  synthetic construct; signal sequence
MRLAVGALLVCAVLGLCLA

SEQ ID NO: 94 - protein, 17 aa, Artificial Sequence
  synthetic construct; signal sequence
MRAWIFFLLCLAGRALA

SEQ ID NO: 95 - protein, 22 aa, Artificial Sequence
  synthetic construct; signal sequence
MFSFVDLRLLLLLAATALLTHG

SEQ ID NO: 96 - protein, 19 aa, Artificial Sequence
  synthetic construct; signal sequence
MKLVFLVLLFLGALGLCLA

SEQ ID NO: 97 - protein, 22 aa, Artificial Sequence
  synthetic construct; signal sequence
MGPTSGPSLLLLLLTHLPLALG

SEQ ID NO: 98 - protein, 18 aa, Artificial Sequence
  synthetic construct; signal sequence
MSLSAFTLFLALIGGTSG

SEQ ID NO: 99 - protein, 27 aa, Artificial Sequence
  synthetic construct; signal sequence
MAPHRPAPALLCALSLALCALSLPVRA

SEQ ID NO: 100 - protein, 22 aa, Artificial Sequence
  synthetic construct; signal sequence
MWATLPLLCAGAWLLGVPVCGA

SEQ ID NO: 101 - protein, 19 aa, Artificial Sequence
  synthetic construct; signal sequence
MQALVLLLCIGALLGHSSC

SEQ ID NO: 102 - protein, 23 aa, Artificial Sequence
  synthetic construct; signal sequence
MQMSPALTCLVLGLALVFGEGSA

SEQ ID NO: 103 - protein, 20 aa, Artificial Sequence
  synthetic construct; signal sequence
MQPSSLLPLALCLLAAPASA

SEQ ID NO: 104 - protein, 23 aa, Artificial Sequence
  synthetic construct; signal sequence
MAPFEPLASGILLLLWLIAPSRA

SEQ ID NO: 105 - protein, 31 aa, Artificial Sequence
  synthetic construct; signal sequence
MLRGPGPGLLLLAVQCLGTAVPSTGASKSKR

SEQ ID NO: 106 - protein, 15 aa, Artificial Sequence
  synthetic construct; signal sequence
MWCIVLFSLLAWVYA

SEQ ID NO: 107 - protein, 17 aa, Artificial Sequence
  synthetic construct; signal sequence
MNPTLILAAFCLGIASA

SEQ ID NO: 108 - protein, 17 aa, Artificial Sequence
  synthetic construct; signal sequence
MWQLWASLCCLLVLANA

SEQ ID NO: 109 - protein, 16 aa, Artificial Sequence
  synthetic construct; signal sequence
MLLILLSVALLAFSSA

SEQ ID NO: 110 - protein, 20 aa, Artificial Sequence
  synthetic construct; signal sequence
MWKRWLALALALVAVAWVRA

SEQ ID NO: 111 - DNA, 1212 bp, Artificial Sequence
  synthetic construct; promoter
gtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggcc

SEQ ID NO: 112 - DNA, 1724 bp, Artificial Sequence
  synthetic construct; promoter
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcc

SEQ ID NO: 113 - DNA, 806 bp, Artificial Sequence
  synthetic construct; promoter
agatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatggatcctgagtaccttctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtc

SEQ ID NO: 114 - DNA, 1003 bp, Artificial Sequence
  synthetic construct; promoter
tggccctggcattcccctatactgggacatagaaccttcacaggaccaagggcctctcctcccattgatgactgactaggccatcctctagctacataggtggtggagcctagagtccctccttgtgtactctttggtggtggttactctgggggtactaggttagttcgtattgttgttcctcctaggggactgcaaaccccttcagctccttgggtcctttctctagttccttctttggggaccctgtgctcagttcaatggatggccaatttccttcttaaatgcccctagcagtaactgttaggtctcaatcccaagacaaatgtctgaggtgcctatttaacagatcaaagcggacctgtcctcaggttaacccagtcactccctgtacctcagtccctacccatcacaattctccagcccatgagcttcgggctgtacttccccaacgggttctcccattttgggtacatggccttttttttttacctttttggttcctttggccttttggcttttggcttccagggcttctggatcccccccaacccctcccatacacatacacatgtgcactcgtgcactcaacccagcacaggataatgttcattcttgacctttccacatacatctggctatgttctctctcttatctacaataaatctcctccactatacttaggagcagttatgttcttcttctttctttcttttttttttttttttcattcagtaacatcatcagaatcccctagctctggcctacctcctcagtaacaatcagctgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 115 - DNA, 523 bp, Artificial Sequence
  synthetic construct; promoter
ttttttttttacctttttggttcctttggccttttggcttttggcttccagggcttctggatcccccccaacccctcccatacacatacacatgtgcactcgtgcactcaacccagcacaggataatgttcattcttgacctttccacatacatctggctatgttctctctcttatctacaataaatctcctccactatacttaggagcagttatgttcttcttctttctttcttttttttttttttttcattcagtaacatcatcagaatcccctagctctggcctacctcctcagtaacaatcagctgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 116 - DNA, 215 bp, Artificial Sequence
  synthetic construct; promoter
tgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 117 - DNA, 198 bp, Artificial Sequence
  synthetic construct; promoter
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgg

SEQ ID NO: 118 - DNA, 142 bp, Artificial Sequence
  synthetic construct; promoter
aaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 119 - DNA, 109 bp, Artificial Sequence
  synthetic construct; promoter
gtcaagtgacggcggctcagcactcacgtgggcagtgccagcctctaagagtgggcaggggcactggccacagagtcccagggagtcccaccagcctagtcgccagacc

SEQ ID NO: 120 - DNA, 93 bp, Artificial Sequence
  synthetic construct; promoter
ctcagcactcacgtgggcagtgccagcctctaagagtgggcaggggcactggccacagagtcccagggagtcccaccagcctagtcgccagac

SEQ ID NO: 121 - DNA, 1080 bp, Artificial Sequence
  synthetic construct; promoter
ttaataaacatttgggcgattcttacggcctctaaagaccaagaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcaccccgctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggaccaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggaccca

SEQ ID NO: 122 - DNA, 969 bp, Artificial Sequence
  synthetic construct; regulatory sequence
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 123 - DNA, 1050 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 124 - DNA, 1089 bp, Artificial Sequence
  synthetic construct; regulatory sequence
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 125 - DNA, 1430 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 126 - DNA, 1318 bp, Artificial Sequence
  synthetic construct; regulatory sequence
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtaaaacaggtaagtccgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggcctctactaaccatgttcatgttttctttttttttctacaggtcctgggtgacgaacag

SEQ ID NO: 127 - DNA, 1579 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 128 - DNA, 1080 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgtgagtatcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 129 - DNA, 568 bp, Artificial Sequence
  synthetic construct; regulatory sequence
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 130 - DNA, 895 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 131 - DNA, 1291 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 132 - DNA, 1147 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 133 - DNA, 1490 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 134 - DNA, 723 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 135 - DNA, 100 bp, Artificial Sequence
  synthetic construct; regulatory sequence
aggttaatttttaaaaagcagtcaaaagtccaagtggcccttggcagcatttactctctctgtttgctctggttaataatctcaggagcacaaacattcc

SEQ ID NO: 136 - DNA, 200 bp, Artificial Sequence
  synthetic construct; regulatory sequence
aggttaatttttaaaaagcagtcaaaagtccaagtggcccttggcagcatttactctctctgtttgctctggttaataatctcaggagcacaaacattccaggttaatttttaaaaagcagtcaaaagtccaagtggcccttggcagcatttactctctctgtttgctctggttaataatctcaggagcacaaacattcc

SEQ ID NO: 137 - DNA, 321 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 138 - DNA, 554 bp, Artificial Sequence
  synthetic construct; regulatory sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctg

SEQ ID NO: 139 - DNA, 393 bp, Artificial Sequence
  synthetic construct; regulatory sequence
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 140 - DNA, 393 bp, Artificial Sequence
  synthetic construct; regulatory sequence
gatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 141 - DNA, 144 bp, Artificial Sequence
  synthetic construct; regulatory sequence
ccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 142 - DNA, 288 bp, Artificial Sequence
  synthetic construct; regulatory sequence
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 143 - DNA, 432 bp, Artificial Sequence
  synthetic construct; regulatory sequence
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 144 - DNA, 199 bp, Artificial Sequence
  synthetic construct; regulatory sequence
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggc

SEQ ID NO: 145 - protein, 22 aa, Artificial Sequence
  synthetic construct; hinge
EPKSCDKTHTCPPCPAPELLGG

SEQ ID NO: 146 - protein, 10 aa, Artificial Sequence
  synthetic construct; hinge
EPKSCDKTHL

SEQ ID NO: 147 - protein, 10 aa, Artificial Sequence
  synthetic construct; hinge
EPKSCDKTHT

SEQ ID NO: 148 - protein, 16 aa, Artificial Sequence
  synthetic construct; hinge
EPKSCDKTHTCPPCPA

SEQ ID NO: 149 - protein, 16 aa, Artificial Sequence
  synthetic construct; hinge
EPKSCDKTHLCPPCPA

SEQ ID NO: 150 - protein, 27 aa, Artificial Sequence
  synthetic construct; hinge
EPKSCDKTHTCPPCPAPELLGGPSVFL

SEQ ID NO: 151 - protein, 27 aa, Artificial Sequence
  synthetic construct; hinge
EPKSCDKTHLCPPCPAPELLGGPSVFL

SEQ ID NO: 152 - protein, 22 aa, Artificial Sequence
  synthetic construct; hinge
DKTHTCPPCPAPELLGGPSVFL

SEQ ID NO: 153 - protein, 6 aa, Artificial Sequence
  synthetic construct; hinge
CCPCPA

SEQ ID NO: 154 - protein, 11 aa, Artificial Sequence
  synthetic construct; hinge
DKTHTCPPCPA

SEQ ID NO: 155 - protein, 11 aa, Artificial Sequence
  synthetic construct; hinge
DKTHLCPPCPA

SEQ ID NO: 156 - protein, 22 aa, Artificial Sequence
  synthetic construct; hinge
DKTHLCPPCPAPELLGGPSVFL

SEQ ID NO: 157 - DNA, 97 bp, Artificial Sequence
  synthetic construct; regulatory element
gtaagtttagtctttttgtcttttatttcaggtcccggatccggtggtggtgcaaatcaaagaactgctcctcagtggatgttgcctttacttctag

SEQ ID NO: 158 - protein, 6 aa, Artificial Sequence
  synthetic construct; cleavage
LSPRGS

SEQ ID NO: 159 - DNA, 663 bp, Artificial Sequence
  synthetic construct; promoter
gacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 160 - DNA, 762 bp, Artificial Sequence
  synthetic construct; promoter
gacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcg

SEQ ID NO: 161 - DNA, 935 bp, Artificial Sequence
  synthetic construct; promoter
gtcgacaaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagatctagagggccccagaagcctggtggttgtttgtccttctcaggggaaaagtgaggcggccccttggaggaaggggccgggcagaagatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggccttaagc